SEQUENCE LISTING (50 sequences)

SEQ ID NO: 1 - DNA, 18686 bp, Homo sapiens
cccagtgaattatttttttattttgtagagacggggtctccctgtgatacccaggctggtcttgaactcctcggctcaagcgctcctcccacctcggcctcccaaagttctgggattacaggtgtgagctaccacgtctggcctgggataactcattgtaaaactggtgaagacctgggaccttcccagtagacaatgggacagagtgattgacaggatgagttctggagtacatggcagaaatgtacagagaagtctcccagagaaaactaactggctggaaacagagcctctcctttcttcttcgagaggatgagagtgtcactgtcttggatgccatagatccccagacccaaccagtcctgcaggacttggccttggaattccagctgctgctgctttttaggaagcccctgctggtcttcaatctgctgcttcagacccaggatgaagctgttggggttgatggcataggcgtagctcccaccatcaggattcttcacgaagacctggatctcggaggggatggtctccagcagatagatgtgagtgtgggagaagatcccatatttggctaaggagcacctgctgctgagaagctgcctctcactgccaggaacctggaaggacagacgctgcaggccagagtagcccctggtcctccggattttctctttaaccttccttatgggctcataagggttcacgatgaggttgaaatctgggtaacccctctgctccactgtcaagtggatgtctcgtgccctctggaagggagagggagaaggagaggtgttaagtccacacttctttgtctgatgtgcaaatgttcccttcatctgtccttatctttacaatagtagcagcagcagcggcagcagcaaatgtttagggagtggtaagcactttccaggctgtatgctgagcaccttccatacccttaatgcattttattctcagagcatctctattagttggttggttctactgttacccccatgttattgatgaggaaactgagcagggttaggtaatttactgaacaccaatcacataggtagtaaggagcagggcagatatgtgaagtgaggttggtcagactccagagtctgtgttctcaccacctccagaggctacactccagggcctgagagccccaggtggctggagagcacctggaaaatgctggcgagggtcaggcctctagctgattctttccagaagaaatataagccttggctgggtgcagtggctcacgcctgtattcccagcactttgggaggccaaggcgggcagatcacttgaggtcaggagtttgagaccagcctggccaacatggcaaaacctatctctaccaaaaaatgcaaaaattagccgggtgtggtggcgtatgcctgtagtcccagctactcggaaggctgaagtgggagaatctcttgaacacaggaggtggaggttgcagtgagctgagatcgcaccactgcactccagcctgggcaacaaagggagactctgtctcaaaaggaaaacaaaaaaagaaaaagaaaaagaaatataagcctggcattgccaagttttctgactagccaagataaaaaatatttgcacaatgttgtgtgtgtttgttttgttttttgagacagggccttgccctgtcacctcaggctgcagtgcagtggtgcaatcttaactcactgcgccttgacctcccaagctgaagcgatcctcccgcctcagtttcctgagtagctgggaccacaggtgcgtgccaccacatctggctaagttttgttcttttttttttttttttttttcagatggaatctcactctgttgccaggctagagtgcagtggcacgatcttggctcactgcaacctccgcctcccaggttcaagcgattctcctgcctcagcctccctagtagctgggactacaggcacatgccaccacacccagctaatttttgtatttttagtagagatgaggttttaccatgttggccaggatggtttcgatctcttgacctcgtgatctgcccacctcggcctcccaaagtgctgggattacagatgtgagccaccgcacctggccaagttttgtatttttagtagagacggggtttcaccatgttgcctaggctggtcttgaactgttgggctcaagggatccacccgcctcagcccctcaaagtgctgtgattacaggcgtgcgccactgcccccagcctacaatggcttttatattcaggaaaaatccagaggcgtatattttccagcccatgggtgggcctcctcatttggccagcgggtccccccgtttatactctatgtaccttactggagcctctgattctgcttctgccttctgctccaagcagttcagtctcctgtcctgtacacatcttgctcatcccagcctcccatcctttacccctcatcatcctttgtcctctgttctgagtgtctatatctccccccggcctttagggctcatttcaactcctaactttttaaaggaatcttctctggtgacttcagttaacatttattctgcttccagacccccagagcccctggttctgctgcctcttagcagagtgcccttagttaattaacctctctgagcctcagttttctcttctgtaaaatggcaataatagtaatgatgataataataataaagatagtgataacactcgaccttcttactttacagaattgcttatgaggatcaactgagacagaccttggacaagagctttgcacactagaaagtgtgatgttaataaaatagatttttgtgatctctgcctatactataggtttggcatcatataataatatgacttagtattttttgtttgtttgtttgttttcagagtatcttgtcttctctggtaattctagcagttttgtgtaagtttgtttcatggatttgcttcctgacaacatagggtgagttctctgagagtgagggtggtgtcttttttttttctttttttcttttttttttgaggcagagtttccctcttgctgtccaggctggagtgcaatggcacgatctcagctcactgcagcccccagctcccgggttcaagcgattcacctgcctctgcctccggagtagcagggattataggcacctgccaccacgcccgcctaattttgtatttttttagtagagatgggttttttccatgttggccgggctggtcttgaactcctgacctcaagtgatccacctgtcttggcctcccaaagtgctgggattacaggcgtgaaccaccgcacctggccgagcgtcgtggtcatgtcttatttctgtgcctgtgttgtcccccagcccacatcccctaccccatccctgagaacagcactggcacagaatggctgattgagctgacttggctggacccaactcacgcaaagaatgatcaggaacacagaactgattctccagtaaattatgggaaaaagaaaggaaaggagaacaagaatttgaaggtaagaaacccaaggctctgagagccaaagaatttgtccaagttcacggagcccatgggtggtgcagctggcccttaaacggtgactctaaagttctgttctccatcaacacagaactgaatgagtaaatactgagtagtttatctgtcgttctgaccttcaagcccttgacagcccagagaggagccattaccttcacgttccagctggagatggggttctccctgttgtcatagcaacagtcctgtttcaggcactgggaggccctctgagcaacgatgtcccatctgtacccttctgccacgttgagggtggggtcggccggatccaggatgatgggcctgtaacacaggaaaagggtgcccaggctcataatggtttgaaggccttcaagccagaaaaaaaccttctcggcaattatttcgtccagccactgagaattgtgatatccacatcccggggatgttcaagagggccccaaaacaccgcaggagagtaacaggatggagacaatgctatttccaagaattcctatctgggccaggcacagtggctcacgcctgtaatcccagcactttgggaggctgaggcgggaggatcacttgaggtcaggagttcaagaccagcctggccaacgtggtgaaaccctgtctctactaaaaatacaaaaaattagccaggtgtggtggtacgcacctgtcatcccagttactcagaaggctgaggcaggagcattgcttgaacccaggaggtggaggttgcagtgagccaagatcatgccaccaccctccagcctgggtgaccgagtgagactgtctcaaaaaaaaaaaaattcttcttctttgggatagtgccttaagtgtttgcaaggaatttagggggagaatgaggccccaagaggcatgctggagtaataacagatgccctttcaggcagggggggccctgtaccttcggagcaggttttgcaagtttaagtttaaggttagagctgctgaattactttggatagtagaaacaccactgccaccaactactgacatttattgaagcacaaggccaaatcctatgcagattaacctattttaatcttacagcaattctataaggtagggactgttatttttattgtttattgttgtggttgtcccctttttacagacctccctaagttcctttttttttttttttttttttttttgagacagagtctcgctctgtcgcccaggctgaagggcagtggtgcaatctcggctcactgcaacttccacctcccgggttcagccaattctctgcctcggcctcctgagtagctgggattacaggtacccgccaccacgcccagctaatttttgtatttttagtagagatggggtttcaccatcttggccaggctggtcttgaactcctgacctcgtgatccacctgccttggcctcccaaagtgctgggattacaggcatgagccaccggtgcccggcctagattgccctaagttcttacagccagtagatgggggctcttacccatctacctcctcaacagagcagaggttgggctgcccccaaaagtctctcctttctataaatattgaataaatattttaaagaatattggcttgaaggggatcttgggcttgcttattaacttggggtgactgcaagtccatgattaacaccagctctttgagtggggcgcagtggctcacgcctataatcccagcattttgtggggccgaggtgggcagatcacttgaggtcaggagttcaagaccaggctggccaacatggtgaaaccgtgtctctactaaaaatacaaaaattagggcatggcggcaggcgcctgtagtcccagttacttgggtggctgaggtgggaggatagcttgaacccaggaggtggtggttgcagtgagccaagatcgtgccactgaactccagcctaggtgacagagcaagactccattaaaaaaaaaaaaacaaaacccaaaaacaaacaaaaaaaacccagccctttgtactccagggcttgggagggagctgcgacttcatctgtgtaatcgaatgtgacacaggctactggaatgaactggactttgggctccaggctggctcctcccaaccctgactgctccctaaaacagtcctgcctgaaagtgccaggctgtcctatggggtggggtcactgatctaaagtggctcattctgttggctggcaggtgacccaacacatggggtctgtgtcccgtgaccaggaatcagactggttctctctggggtttggacatggggatgatggagatcccttatacctgggaggcagaggggagccggacaggtgtggagagagaagcagaggctgaggggaaaagtgctgtagttcatgagagagagtgacagagagagagagggagtctcaggtcctgatgctttctcgatttttctggccatggttataataaatcctcttttcatgagcagacctggcttttcttccttacaaaagagacccaatactacataacagaacactaaatactgtgatgttgcttacacaggcccagagcttctaacacagagagttgtctgggttgggcttcccctgaagcagatccccagataaggatttggcttatttggaaagtgatcccaggaagcacaaataagggagtgagacagtgagacagataagggaaggcagacaacaaagagtgggtattgaagtaattactactgtggtccactggaggcgatctttaggaaagagtatggaatggggcccagtgcggtggctcacgcctgtaagcccagtactttgggaggccaaggcaggcgcatcacctgaggtcaggagtttgagaccagcctggccaacatggcaaagcactgtctctacaaaaatacaaaaatcagctgggtgtgggttgtaggcgcctgtaatctcagctactcagaagcctgagggaggggaatcactcgaacccaggaggcagaggttgcaatgagtggagattgcgccactgcactccaccctgggtgacagagcaaaactctgtcttaaaaaaaaaaaaaaaaagttacgcaatgagacccagattccttccatcttttgggtgagggagctgtggtatttatccaccaactcccaccagatctggtggagggctcttctcagagtgggtctgctggcaaagggatgcaggtgctgacagctggaagtggaaccaaccttccctagaacggtaaaggccgagaggatatgaatgggacgctgacagcacatgctccaatataagcatttactcagcatctgccagatgctagatattgtggatccttaacacagattttctccaacccttacatcaatcctatgtcgtaggttccagtttactcctgtcttatggataagggattgaggctcatagcagtaggaaaatggctgagctgggatttaaacttgagtctccaagattctttccattttcccatgctagctcctacaaagcactgaaatggtcttcagagagcaagaaggccaagctaaggtggagggtggcttaggaaacaaggaacaggcttctacccgccccatggccccccagtgactttgataagccaccaacttgagatgccagctgtgttggtgtgggaggtcgatgctcaactatggtggcagccttactgcttaatacagccaatccataaaactctatgccacgttacactagcccgtctctctttttttctgtaagatttggtgggcaaacagtggtccagtacctctcttttttgagctgttttctgacacaatcctcaatgattgcattgtggagtgtgtagtacttggtccagtagatacagatgacttcatactccaggagcaggtccatcacagtggtgaagccttcgtccaacatgaaattctcgtcttcttcagtacccatttcccaggcatagatggttagaagttcaagagcatagagagggggcagattggctctgggggacctggctttcacatactgttgaaagagatgggaagacagagagagagagaccctaagattctgtgtaaggatggcgttggaccagaagctgagaaatccaagttctagtcctggctctgctgcctactggctgtgtgaccttggccatgtcacattccatctctagacataggtttcaccattttagaattaaagggctgagttagtccattgggcttcaatatatgctcaacagatgtaactcctggaatgaacttatgaacaggtatgagcagattgcgctcggtcacaaacattctagcctaagcagtcccatttaaatctattttaatgtattgagctgctgagaaatgtttcttaaaagaaaggaaaggtttccttagatagagggaaaaaatggtaataggtgagattctgtagtgaagattggggttgcctctctagcagacattctttccttccccattgcatagtagccatgatgtttggaaggggttgacccccacccgcagctccaggatgggccctgactggcacagtcctatcagcataaacctttcccccttgcaagtgattgcttcaagagaaggcatgcgatctaagttggctcaactagagtaaagcctagttcttttgttcagtggttgaaagaagagaagtcctcccatgtgtgaatatgagggcaggaatagttgtggccattttgccaccaagagggaaacaagccaaaggaccaagctgacatatagaggaatgagaagctgagagaattccagagaaacagagccagtgccctcaccagtgcctgaaacccgccccccccgcccgccccccctactgtttaggggttttcctacataagctgataattccctttattgtttaagccagtttgagttggtttttgtgtttttctcttacttgcaaaaggaagcatcttaactgacagtggtcacttcctgtgttaagaatctcactgaggccgggcgcagtgcctcccgcccgtaatcccagcaccttgggaggccgaggcgggtggatcacttgaggccaggagttcgagaccagcttggccaacatggtgaaaccccgtctctactaaaaatacaaaaattagccaggtgtggtggcaggtgcctgtaatcccagctacttgggaggctgaggcgggagaatcgcttgaacccaggaggaggaggttgcagtgaaccaagatcatgccactgcactccagcctgggcaagagtgagaacttgtctcagaaaaaaaaaaaaaaaaaaaaaaagaatttcactgatacattgatatttagccagaaagtgaaaaattttggtcaaatattcagtagtttgatttgtggaagtaaaactgacatattttcctatggccatcatatatgaatattagctcccatcccaaagtcagaggagaaaaaaaaaagctataacactttggccttgaaacaaacacagaatcatcttgtataattctcatctcccaaataaatgaaaccttgggcctttcctataggtcactgacttactaacaaaaaaactttatccttgaccttagaaattcaattttgaattaaaatgtatttataaggctgggctcagtggctcacgcctgtaatcccagcactttgggaggccaaggcaggtggatcacaaggtcaggagatcgagaccatcctggctaacacggtgaaaccccgtctctactaaaaatataaaaaattagctgtgcatggtggcgggcacctgtagtcccagctactcgggaggttgaggcaggagaatggcatgaacccgggaggcggagcttgcagtgagctgagattgcgccactgcattccagcctgggcgacagagcaagactctgtctcaaaaaaaaaaaaattataagttaattaatttgttttgtaactttttgcaaattcatccttaacactcaattttgtaaaccataactaataacattataattttgttagaaatttaaactcatggtaatataacttattttcttcaatattagtgaaatgttacattcttaaaacttagatgatatataaaatgtaggatgcctgtaatctgtaagatgtaagccttagtgaatagataaaaaaaatacccagctcaacttacttcacctccggggctcaagtgatccttcctcctcagcctcctgagcagctgagactacaggaacccatcaccacccccagctaatttttgtatttttgtattttttgtagagacggggtttcactatgttgctcaggctggtcttgaacccttgggctcaagtgatcctcctacctcagcctcctaaagtgctgaaattacaggcgtgagctaccacatttggccggtaattgttttttctttaaatccctgttatattttattttattttttgagacagagtcttgctctcttgcccaggctggagtacagtggcgcgatctcggctcactgcaagctccgcctcctgggttcacaccattctcctgcctcagcctcccgagtagctgggactacaggcgcccgccaccacgcctggctatttttttgtatttttagtagagatggggtttcactgtgttagccaggatggtctcgatctcctgatctcatgatctgcccacctcggcctcccaaaatgctgggattacaggcgtgagccatcatgcccagcctattttatttttattggatcaaatctttccttctgctgtctgattcccttgtaacttctctgcaagttctatataataaatgatttttgtctctgagttctgttttgcagttggagtttccgttatctcggaggaagattttgtttccaaaactgactgagctgctgcctgagaaatgtccctgagggctgtttacactcacacatgccctcttttcgactcctggggtggaattaaagaaatccttccccttgttagtccttaacctgctgtgacgaatggccacagtacccggcgaagctgacatctgccaggcctagactgtaattgaagggtaacaagaacatgtggggtgggtgtggtggctcgtgcctgtaatcccagcactttgggaggccaaggtgggaggatcgcttgagaccacttcgaaaccaggctggtcaacatagtgagaccctgtctctacaaaagaaaaataaaaaaaattagctgggcatggttgtgcatgcctgtagtcccagttacttaggaggctaaggtgggaggatcacttgagccagggaggtcttgatccagggaggtggaggctacagtgagtggtgattgcaccaccgcactacagcctgggcaacaaagcaagatcctgtctcaaaaaaaaataataaaataaaataaaaataactagtttactgaaatactgtagagcacctatttttctccttccttctctgtttctttctcctcttcctcctcctctcctccttttccaccttcttcactgacggtttcactttacatttcaaaagactagcattctctccctctctacctgcttccatttcccctggctgcagcttcctgagatgaaaatcaggccctgcctttcctcctcaccctcttccctctccttgcccctgccatcctggcagccccctcacctgctggtaccagtgtttcaccaggcgcaggaggctcttcagcttagttggccgatgtttcacgaaatttctctgcagctcgctgaaggatgggcagaaatttccaggaccaccgcaggccttgatcaggctcacatagacctcagggggtggctgggagttgggaagagaaggccctgaggagtgaaggagcaaaggaagagattggggattgaggaagccagagggtggtaagtagtattttggggagagcctgcctttacattggaagtatccctccagggacacttgagacctcatatcttcagaaaccaaaggtttgccttgtctaattgtcctttttgtgaaattcttactctggagaaccaacattgggctttccatgcatcagacccctccctggatgtcctcaatgtccctcattttctttaatatctgttttgtatctctttcaatactcatagggaggccgggtgtggtggcttaagcctgtaatcccagcactttgggaggccgaggtgggcggatcacctgaggttaggagtttgaaatcagcctggccaacatggtgaaatcctatctctactaaaaatagaaaaattagccaggcatggcatggtggcgggctgtaatcccagctactcgggaggctgaggctggagaatcacttgaaccctggaggtggaggttgcagtgagccgagatcacggcactgcactccagcctgggcgacagagcgagactccgtctcaaaaaatatatatatatataaactcatagggagaaccaggaaccagttccatggtacacatcactgagacatcccagcactctcttccttgctaaaatcagatcctgtctcttataccttctccaaagagcaactccacgcagccagccactacagctgataggagttagcacatgagatgaaacctctaggatctgggcttgcagcatgtaggttttacttcaaacacctaggactctgcccgaggacaaacaacaggggccagtgagttagtgctcccaaagcagcccccaaccagtgacggctgggaagctggtgagtaagcacgccagcgtcccacttgggatcccgaagaggttcgctccataatgggactgtgagttctcccaggacatgaagctcagggtggtgacttgcccatggacgcacgttgtattgaattcttgcctttcccatctcagttccatacttccctaccggtgtttcctgggaggaatacccaaataaactgcgtatcctcacatccatgtcttaggatctgcttctataaaatgcagtgacagacccccactcccacttttagctccttcccctcactgggtgtcagagcctcctgaatgttgtttccccagaaagtcacttcctgagcaccctttctgtggttacccaggccaccccattctttttctctgagcatttacaccatttgcaatcatgtttttttgtttgtttttttgttttttgtttttttgagacggagtcttgctctgttgcccaggctggagtgcaatggcgcgatcttgactcactgcaacctccgcctcctgggttcaagtgattctctgcctcaccctcctgagtagctgggattacaggcgcccaccaccacacccagctaattttttgtatttttagtagagatggtatttcaccatcttggccaggctggtcttcaactcctgacctcatgatccacctgccttggcctcccaaagtgctgggattacaggcgtgagccaccgcgcccaggctgcaatcatgtatttatctctgcaattctttggtcaatttctgtattccaagttgagcaatatgatagccagagttgagttaatcctgtgcactgcaataacccagccccaagcataggacacagatgaaatgaataaatgtgggtgtgtgcacgtggccatgagtaaaggtgagacactgggaagtctcctggggtgggcaagtgtgtgagtcgggtgagcttcccctccccttacccagggctctgtaggcaggcacaatggtgaccgtgatgggctccgcagtccccctggtctggatggtgaagacgagagcatcggggactctctgctccatcctcaggtcctcgagcccgaggtccagcaggtcctggctttgccacatggttttccatatcagcctcagaacatctttgtgatgcttggctgcctcctggaagctgtggaaacagctcagaaacgccaccagctccacctctctggtgctcctgagaaccgtgccattcccgaaggagcccacctgcagaacacagagccccgtcaccctgaggcccactgccatgagccaggcaacccctgcggcacttcacatgcactgtctcactgaatgctcaccacccgctgagggatgggttgtgggtagtactaatacccacactttacaggtgagaaaactgagatgtctggaggatctcgagacatgggatggagaaatggggcagcctgaaaaagatcttgacacttccctgtttcttagctaggtaaatgttctgactcctaagaccttcatctctaaaatgaaagcaatggcagaatatgagtcactgggctactgggaggatcagatgaaataatgtatgccaagggtttggtggggtgcctggcacacaggaagtcagcaacacatttgtctttctcctcattagtcatttgtctcaggtctcgcagtttgcagccctcagagcccagactctttttttttttttacacagggtttcactcccgttgcccaggctgggctgcagtggcatgatcttggctcaagcaatcctcccacctcagtctcccaagtagctaggactacaggcctgcgccaccatgcctggctaatttttgtattttttgtagtgactgggttttggcatgtcgcccaggctggtctcgaactcctggactcaagcgatttgcccccttggcctccccaaatgcccagcttcttatctactacactgcattgcttctgagacccagtcttcttacccacagagtaaggcctactccagaggaatggtagcatctactccatagggttgtttgaggattctgggggtggtggtggggctgaataatttcatgacattataaagcaaggagcaagtttgcctggtggatttaagagccataggctgtgaaatggagcagcctgaaaaagatcttgactcttccctgtttcttagctgggtaaacattctgactcctgagaccctcatctgtaaaatgaaagtaatgttggaacaagagtcactgggctactgggaggatcagatgaaataatgtatgccaagcgtttggtggggtgcctggcacacaggaagtcagcaatacatttctctttcttctcattatccagctgacttagacaatctcttctgctgccggggggtcagttttcctgttagtaatggaaggaggtcgagaagggatttttcgatgttttgagattctagatgagctctccaggccctggtgcctctggtatgcaaagcgccacaactctctggcctcctctccttctcattctgtgttgggaggtggaattgctagggaggcatcagacccccagaaggctacttttcttggtggttggagtcactgcacaagaatcagattccagctgttgccggatgatataacatgccctgtgtgacatgagcacggatgctggatgacatgagcacgccctgtgtctggcacccataagagccctgtatacatattattccttttaatctttacttcccagccaggcatgatggctcatgcctgtaatctcagcactttgggaggccaaggtgggcggatcatgaggtcaggagttcgagatcagcctggccaacatggtgaaagcacatctctactaaaaatacaaaaattagccaggtgtggttgtgtgcacctgtaatccgagctactggggaagctaaggcaggagaatcgcttgaacccaggaggcagaggttgtggtgagctgagatctcgccactacactccagcctgggcgacacagcgagactccatctaaacaaaacaaaacaacaaaaaaaaacgccttcacttcctgtattgaggtgggtactaggagaattatctccaatgtacagatgagaagactgaggcagtgaggagcttgccatggtctcccagccagaaggcagcagagtcagagaaagagccagaactcaagtaggggatggttcttccgcctctttcttcctcgtgacccaaggctgtgagccaggccctgcttccaagggaccatgggcattcagacccagcacctccaccccctgggagagggttggagatggcaccctgtgtgtgaatgaaggacacattccccccgaaggccagtctagacttccctttctttttgagacagggtcttgctctgttgcccaggctggagtgcaatggcacgatcttcactcactgaagcctcgacctcctgggcttaagcaatcctcccacctcagcctccggagtagctggaactacaggcatgcaccgccatgcccagctaattttttatatatatttttgtagagtcgggggtctcaccattttgcccaggctggtctcgaattcctgggctcaagcaatcctcccaccttggcctcccaaagtgctgggattacaggcgtgagccactgtgcctggcgtagacttccctttctgcctttcccactggtaagatccaggcagagagaaaagaacacatccagaacaggcttcctgatttgcttcagttttaaacctatttaagcacaattcatttgaagacgagcattcccttatgtggaccagctccgctcactagattttcagagggtggggaacgggagggtttagactctaaataagatatattattcctcattagtcatggctcactggggattgctaccatttagtggtttcaagaggaaaagtggctatgatgtgcaggaagtatttgaggcttaggtaaatagcatttcctgccgtaaaaactcattcttggccgggcgtggtggttcacacctgtaatcccagcatttggggaggccgaggtgggtggatcacttgaggtcaggagttcgagaccagcctggccaacatgatggaaccccgtctctactaaaaatgcaaagattagccaggcgtggtggaacacacacacacacaataaacttattcttgtagcatcctcctcttaccagacattccttattccctccactttattctcaagtttgttggggagggggtggtctaggagggtctgccagagtcagacttgagtcccaggcccagaagaaagcttggcaagaattataatgatgcttctgtttctgagcatttatcgtgcatgaatcattcagcctgcattgcaccactggcttggtgagggagagatgatgatgatgccccaatcacagatgaaaagaatgtgggtttgagaaagaagagtgggaaggtcaaaaccacacagctggtaaaggaagaagcgaagagtctgtctaatatcaaaatttgtggtttctcacttataaattgtatggcctcaggcttcatatgctggggataggtgcaaagaacggataggggcccagggcttctagatccctctgtgagttggttgcccctagagagagaacttgagcactgggctgggagtcaggggtcctaattctagtcttgcctttgccaccaactcgctgtgtgaccttggggtatctctcaccctctctgtgcctcggtttacttaattttttaagtgagtcattggtgagactgtctgattctccaaagtctttctagctccgaccctcttgaattttgatcttagctagcacaggcggtggcatgaaacagctggaggaagggcttgggggaaaatctcagtgaggggggcttgggtcccttgagtcaagttcaaatcccagcttcaacactgtgtgaccttgggcaagtcacctcacctctctgagcctccattaaaaaatcattttaggccgagtgcagtggctcactgtaatcccagcactttgggaggccgaggagggcagatcatgaggtcaggagatcgagaccatcctagccaacacggtgaaaccctgtctctactaaaatacaaaaaattagccgggcgtggtggtgggcgctgtcgtcccagctacttgggaggctgaggcaggggaattgcttgaacctgggaggcagaggttgcagtgagctgagatcgtgccactgcactccagcctggtgacagagcaagactctgtctcaaaaaaaaaaaaacaaaaacaaaaatcattttattggccggccagacatggtggctcacacctgtaatcccagcactttgggaggccaaggtgggcatattcttgatcccaggagttcgagaccagcatgggcaacattggtgaaaccctatctctacaaaaaatacaaaaattagctgagcatggtggtgcatgcctgtaatcccaagctatttaggaggctgaggatcacctgagctcagggagattgaggctacagtgagtggtgattgtgccactgcactccagcctgggtgacagagcgagaccctgtctctaaaaaaaaaaaaagtcattttattatcaaaaagttaaaacacatagaaaaatagatgatacaactaacactcatatacccaactagttgattcaacaagtctaaattcctggctacatgtgcttaatctaagccacgttaaagtaaattataggcaccaaggaactttgtccctaaaatacttcagtgttcgtgtccaaacaaaagacaaccttctgataaccacaaaactactgttacacctaacaaaatcaacaatgatttcccatatcagatatatgtactatctgatatctaggccagttgtccccaaactatattttacagatggttcattcacactaggatccaaccaaggacctcgcattgtattggttctgagcctccatttgctaatctgtaaactgggcaaagaatagtacctatctcacagagtgcaagaattagagatagtgtatgttaagaggttagtccagtgagcccccaaatgtgagctattattactagactcattctactcaaggcagtggtgcagtggggtgtagacagctaggtgggatggggtattctgagatgtcatcagcatgggctagggataaggaggacaggggtccccagcatccccagggacgtggactctgatactgggttttgtcttgcgtgggggctggaggagcccagtcttaccttgactaccttcagcacccgcacatcctggtccagcccacgcttcccctggaaatgctcctgcctcagaaactcctccacggtccgcacagcgtctagcacctcttccttccactcccggtggggctgcagccactgagccacgaaggagtccagcctggaggctggtgtgctatacagttcctgcatcagtgccatctctgt

SEQ ID NO: 2 - DNA, 1826 bp, Homo sapiens
acagagatggcactgatgcaggaactgtatagcacaccagcctccaggctggactccttcgtggctcagtggctgcagccccaccgggagtggaaggaagaggtgctagacgctgtgcggaccgtggaggagtttctgaggcaggagcatttccaggggaagcgtgggctggaccaggatgtgcgggtgctgaaggtagtcaaggtgggctccttcgggaatggcacggttctcaggagcaccagagaggtggagctggtggcgtttctgagctgtttccacagcttccaggaggcagccaagcatcacaaagatgttctgaggctgatatggaaaaccatgtggcaaagccaggacctgctggacctcgggctcgaggacctgaggatggagcagagagtccccgatgctctcgtcttcaccatccagaccagggggactgcggagcccatcacggtcaccattgtgcctgcctacagagccctggggccttctcttcccaactcccagccaccccctgaggtctatgtgagcctgatcaaggcctgcggtggtcctggaaatttctgcccatccttcagcgagctgcagagaaatttcgtgaaacatcggccaactaagctgaagagcctcctgcgcctggtgaaacactggtaccagcagtatgtgaaagccaggtcccccagagccaatctgccccctctctatgctcttgaacttctaaccatctatgcctgggaaatgggtactgaagaagacgagaatttcatgttggacgaaggcttcaccactgtgatggacctgctcctggagtatgaagtcatctgtatctactggaccaagtactacacactccacaatgcaatcattgaggattgtgtcagaaaacagctcaaaaaagagaggcccatcatcctggatccggccgaccccaccctcaacgtggcagaagggtacagatgggacatcgttgctcagagggcctcccagtgcctgaaacaggactgttgctatgacaacagggagaaccccatctccagctggaacgtgaagagggcacgagacatccacttgacagtggagcagaggggttacccagatttcaacctcatcgtgaacccttatgagcccataaggaaggttaaagagaaaatccggaggaccaggggctactctggcctgcagcgtctgtccttccaggttcctggcagtgagaggcagcttctcagcagcaggtgctccttagccaaatatgggatcttctcccacactcacatctatctgctggagaccatcccctccgagatccaggtcttcgtgaagaatcctgatggtgggagctacgcctatgccatcaaccccaacagcttcatcctgggtctgaagcagcagattgaagaccagcaggggcttcctaaaaagcagcagcagctggaattccaaggccaagtcctgcaggactggttgggtctggggatctatggcatccaagacagtgacactctcatcctctcgaagaagaaaggagaggctctgtttccagccagttagttttctctgggagacttctctgtacatttctgccatgtactccagaactcatcctgtcaatcactctgtcccattgtctactgggaaggtcccaggtcttcaccagttttacaatgagttatcccaggccagacgtggtagctcacacctgtaatcccagaactttgggaggccgaggtgggaggagcgcttgagccgaggagttcaagaccagcctgggtatcacagggagaccccgtctctacaaaataaaaaaataattcactgggaaaaaa

SEQ ID NO: 3 - DNA, 1584 bp, Homo sapiens
acagagatggcactgatgcaggaactgtatagcacaccagcctccaggctggactccttcgtggctcagtggctgcagccccaccgggagtggaaggaagaggtgctagacgctgtgcggaccgtggaggagtttctgaggcaggagcatttccaggggaagcgtgggctggaccaggatgtgcgggtgctgaaggtagtcaaggtgggctccttcgggaatggcacggttctcaggagcaccagagaggtggagctggtggcgtttctgagctgtttccacagcttccaggaggcagccaagcatcacaaagatgttctgaggctgatatggaaaaccatgtggcaaagccaggacctgctggacctcgggctcgaggacctgaggatggagcagagagtccccgatgctctcgtcttcaccatccagaccagggggactgcggagcccatcacggtcaccattgtgcctgcctacagagccctggggccttctcttcccaactcccagccaccccctgaggtctatgtgagcctgatcaaggcctgcggtggtcctggaaatttctgcccatccttcagcgagctgcagagaaatttcgtgaaacatcggccaactaagctgaagagcctcctgcgcctggtgaaacactggtaccagcaggcccatcatcctggatccggccgaccccaccctcaacgtggcagaagggtacagatgggacatcgttgctcagagggcctcccagtgcctgaaacaggactgttgctatgacaacagggagaaccccatctccagctggaacgtgaagagggcacgagacatccacttgacagtggagcagaggggttacccagatttcaacctcatcgtgaacccttatgagcccataaggaaggttaaagagaaaatccggaggaccaggggctactctggcctgcagcgtctgtccttccaggttcctggcagtgagaggcagcttctcagcagcaggtgctccttagccaaatatgggatcttctcccacactcacatctatctgctggagaccatcccctccgagatccaggtcttcgtgaagaatcctgatggtgggagctacgcctatgccatcaaccccaacagcttcatcctgggtctgaagcagcagattgaagaccagcaggggcttcctaaaaagcagcagcagctggaattccaaggccaagtcctgcaggactggttgggtctggggatctatggcatccaagacagtgacactctcatcctctcgaagaagaaaggagaggctctgtttccagccagttagttttctctgggagacttctctgtacatttctgccatgtactccagaactcatcctgtcaatcactctgtcccattgtctactgggaaggtcccaggtcttcaccagttttacaatgagttatcccaggccagacgtggtagctcacacctgtaatcccagaactttgggaggccgaggtgggaggagcgcttgagccgaggagttcaagaccagcctgggtatcacagggagaccccgtctctacaaaataaaaaaataattcactgggaaaaaa

SEQ ID NO: 4 - DNA, 2123 bp, Homo sapiens
caagagttggtaagctcgctgcagtgggtggagagaggcctctagacttcagtttcagtttcctggctctgggcagcagcaagaattcctctgcctcccatcctaccattcactgtcttgccggcagccagctgagagcaatgggaaatggggagtcccagctgtcctcggtgcctgctcagaagctgggttggtttatccaggaatacctgaagccctacgaagaatgtcagacactgatcgacgagatggtgaacaccatctgtgacgtcctgcaggaacccgaacagttccccctggtgcagggagtggccataggtggctcctatggacggaaaacagtcttaagaggcaactccgatggtacccttgtcctcttcttcagtgacttaaaacaattccaggatcagaagagaagccaacgtgacatcctcgataaaactggggataagctgaagttctgtctgttcacgaagtggttgaaaaacaatttcgagatccagaagtcccttgatgggttcaccatccaggtgttcacaaaaaatcagagaatctctttcgaggtgctggccgccttcaacgctctgagtaagcattgctgggtgtcaggagagaaaagccaaagaagcgggtgccagacagctctgtgcaacctctaggccatgagtgggatagataccactgctgctttaaaaaatgggagaccatagaccctcaggagagaagaatcccttctaccctggactcgctctcttctctggaactaacttctcccccataccctgattgtctttggagaaaatgttctggattctagaatctaaggcagagccttttaagccatactgtacacataaatcacctggaaccttgttaaaatgcagatcctgactcaggaggtctgagttagagcccaggatttcatatttctagccagctccatgatgagctgctggtccgcagatcatgcttgcaggttttgaccagagtcagtgttggttagagtaagaggatgaggcagacatctgggaaaagtccagctggggcaagcatttgaagtctgccttcctaccaggtcaaaatcaaggcaacgaccttccatagataactatcaaagcttgagggggtgccttgaacccaactcctaaatccctaagacctgcccacctcttgtgtctcctgtctcagcaaacattcccacactcttgcatattgttaaagtaacctctgcttaccaggcttctggtttaataaaagatggctagagtgactccatcttaaagcaagtagctaggcactcaaaaggaacctacaggcttaatacttgggtctgaaaatagccacagtctaagctgaccaccaattataattgcagaatatttaaggccatacaaaacatctcccactaagcctacaaaatgtccaggtgtcctaaaagttcagcccacttaaaggcagcattaatgagcaggtttaggttgaaggattaatggtcatcaataccactgttaagaagaaaattcttggccaaattgaatttaatggagtttaactgagcagacaattcacaaatctagaagcctcctgagccagagtaggttcagagagtcttgaacacagccacgtggtggaagaagatttatggacaggaaaaggaaaatgatgtactgaaaatgaaagtgaggtacagaaacagccagactggttatagctcagcattggccttatttgaacgagatttgaacagttggccacctttgattggccgaaactcagtgattggcacaagagtaggttgcagtctgtttacacatccttttaggttatagttcaccatgtacagagaaattttaggccaaacttaaaatatgtaaggaggcagctttaggctaaacttgatttaacagcaccaataccccctacctttagtgagcacatctgcacattccaattttaatgacagctccttagaatttcttatcaacgaagacactaacaaagaatggcgcattcctccttctcctttctgaggatgccctaccctgtaacaaagtcgtttctaataaatttgcttctttcaccataaaaaaaaaaaaaaaaaaaaaaaaaaaaaaaaaaaaaaa

SEQ ID NO: 5 - protein, 514 aa, Homo sapiens
MALMQELYSTPASRLDSFVAQWLQPHREWKEEVLDAVRTVEEFLRQEHFQGKRGLDQDVRVLKVVKVGSFGNGTVLRSTREVELVAFLSCFHSFQEAAKHHKDVLRLIWKTMWQSQDLLDLGLEDLRMEQRVPDALVFTIQTRGTAEPITVTIVPAYRALGPSLPNSQPPPEVYVSLIKACGGPGNFCPSFSELQRNFVKHRPTKLKSLLRLVKHWYQQYVKARSPRANLPPLYALELLTIYAWEMGTEEDENFMLDEGFTTVMDLLLEYEVICIYWTKYYTLHNAIIEDCVRKQLKKERPIILDPADPTLNVAEGYRWDIVAQRASQCLKQDCCYDNRENPISSWNVKRARDIHLTVEQRGYPDFNLIVNPYEPIRKVKEKIRRTRGYSGLQRLSFQVPGSERQLLSSRCSLAKYGIFSHTHIYLLETIPSEIQVFVKNPDGGSYAYAINPNSFILGLKQQIEDQQGLPKKQQQLEFQGQVLQDWLGLGIYGIQDSDTLILSKKKGEALFPAS

SEQ ID NO: 6 - protein, 255 aa, Homo sapiens
MALMQELYSTPASRLDSFVAQWLQPHREWKEEVLDAVRTVEEFLRQEHFQGKRGLDQDVRVLKVVKVGSFGNGTVLRSTREVELVAFLSCFHSFQEAAKHHKDVLRLIWKTMWQSQDLLDLGLEDLRMEQRVPDALVFTIQTRGTAEPITVTIVPAYRALGPSLPNSQPPPEVYVSLIKACGGPGNFCPSFSELQRNFVKHRPTKLKSLLRLVKHWYQQAHHPGSGRPHPQRGRRVQMGHRCSEGLPVPETGLLL

SEQ ID NO: 7 - protein, 172 aa, Homo sapiens
MGNGESQLSSVPAQKLGWFIQEYLKPYEECQTLIDEMVNTICDVLQEPEQFPLVQGVAIGGSYGRKTVLRGNSDGTLVLFFSDLKQFQDQKRSQRDILDKTGDKLKFCLFTKWLKNNFEIQKSLDGFTIQVFTKNQRISFEVLAAFNALSKHCWVSGEKSQRSGCQTALCNL

SEQ ID NO: 8 - DNA, 15312 bp, Mus musculus
cactcactgaagataactagacagaaaacatcgtgagactacctcacgctgtccattgaccaggagcaatcctttgtgcctcacatctttgggactgcggtaggcatcagagaaatgtcacctcgatgggccctgcagctggagagtctgaccctggatatggccttcagctaagacacctgcacagatgatgagcagcctcggggagactagctgaggtcggtagttaggaatgtggagtgtggatgttgtgaccctcagcataagcaggaataacctagcctaaccctatcgctcgagacgccactagtaacggagacactatgcgtgattaagaatgaaataaatgcatttgccactgccagatcaccaggcagatgagattatgtggccaaatcgctgccactgaaactgctgaatcttactctgttgtggtaaacaacatcctgtgctcacaacagagccggtggggagactgcatccttaactcctgttcccttctgccttgaagaaggtaaactggatctccctgcagtttcactttcaattcttgtcaaagcttggtttctgtttccagtttctccaaggagggaggggcagagcgcttttcttctttccatgcaactctccatcccattgtctcctccagagaacgctgcaggaagcttctcccatcagagctgagcagaagctcagaggatttatctagaggacaggctggcccagctgaaatggacccgttccccgacctgtatgcgacccctggggacagtctagaccacttcctggaacacagccttcagccccagagggactggaaagaggaagggcaggatgcctgggagagaatcgagaggttctttcgggaacagtgcttccgtgatgagctgctcctggaccaagaagtcagggtgattaaggtggtgaaggtaagcacacagggttctgagtttaaagctcacctctgccctgtggggcttgtgaacttgagaggtgggccatttcaccccctctgagtctgtgtcctcttcttaagatgggtttagtagaagcagtaaatgggataaagcatagaaaatttctgtgcatttaaaattagccttgcaatgctgaaggacagaggtggtaatgataatttgggattctggttcctgaatggaccatagtcaccggtttccataaaatgtgtgcctttgcttcctgtctaagaaataatacttggccaggcgtgtgtggtcctgcatgcttttaatcccatcactcaagaggtagaggcaggcagatttctgagttcgaggccagcctgatatagagtgagttccagggcagccagggctgcatagagaaaccctgtctggagaaagggaagggaagggaaaaagggaaaggaggaaaagggaaagggggaaaggataggaaagaaaaaaaaaaagactactactttacttctaagtgaaggaaacctattgttctctaagttagtagtattggtcatgttagaaattacctagcaagactacttggtaaaccattgggattctaagggctgcctgtgtaagtaattctacggctagctgtttctcccagaaaaacacaaacttacagcccaccccctccaatcaagaccatttggatcagagctctgggtgttgtttattctgctaaaatgaatgacaacttaagtggtttttttttttttgttttttttgtttttgtttttcgagacagggtttctctgtatagtcttggctgtcctggaactcactttgtagaccaggctggcctcgaactcagaaatccgcctgtctctgcctcccaagtgctgggattaaaggcatgcgccaccaccacccggcatgacaacttaagttaacttgagtaagtcaactttacctgggaactctgagccatggtagcagaggttaagacgggagagaaagagcttttgatgaagaaaaatctcaacaaaccttcaaaaccagagatcagggagggttcttagccaaaaagctttgaagacagaaatggaaactcccacagctgctgcaaaaggatacatcgtatcccatttgtgttctggttcctgcaaatttctgcctgacctaggtcgattgcttgtagcagcctcttggcttctgggacttgaaagaaacaaacaacaaaaaaaggaagtccagagctgggtgtggtagcgttcacctgtaaccccagcagtcgggaggttgaggcaggaggattaccagtttaagactgttaaacctgttcttatagcaagttccaggccagccgtgactgcatcagtgaggcactgtcgaggggtcaagaggaggagctgaggtttagactctctcatcttctttggttttacctttttttttttttaaactagcagaaaaaggattttgtttcattatagcatttttcaagtagcaatttgttttgggctagggatgtagttctgaggttggtatgttttgtgcctagaatggtggctacctgtaacttaactccagaggcgctcccttccacctctcttctggcttctgaggatagacaccaggcacacctatggtgtatgtgtacatgccgacaaagcagccatacacataaagtgttgcccttgttgagatttcagaacatttgggtctctcagttttggatcagggatgctcagcccataacgcccacagcacaggtctctcttaaaggactgtacagagaacttaccatgaatgggtctggcttaactctcaacttcaaagcatcaaatctcaggagaggtaaatctgccattgtcaccttccacagggaggctcctcgggaaaggggacaacgctgaaccacagatctgaccaagacatgattctgttcttaagctgcttttccagtttcgaagagcaggcgagaaaccgggaggtcgtcatcagcttcattaagaagaggctgattcattgtagcagaagcctggcctacaacatcattgtccttacccacagagagggcaaaagggcccctcgctccctcaccctaaaggttcagtcccggaagactgatgacattatttggatggatatcctcccagcttacgatgctttgggtaaatccagtggaagtttcagtttaaagaaagaattctggggtcttcctctagcaagattcctctggaagattgctttgtagatagataggtgggactgaaatcatactgtgaagatggatagacttccataataaataaatatccttgcatgggttcccatgtcccagtgttacatggggaggaaggatttatcataaagaatgaattcctacccaccctctggaaaacaccaggttaggcaaacattccaaggtctttagggtccaacagaactgtattagaccctttaaaacaaggctctcaagctgggtggtgatggagcaagcctttattcccagcacttggaaagcagaggcaggtggatctttgtgagtttgagaccagcctggtctacagagcaagttccaggacagtcagggctacacagagaaaccctgtctcaaaaagtaagtaagtaaatgaaataaataagcaaacaaataaataacaagactctcgtattttacaacttgtattcctcttgggcccacaccttagcatgctcaaaatcgagcatactaacttactacatgggataatgttgagtcatcagaactgatatttgctcacatttgagataccagacggcgattgcctgtgcttgcctttaagtttacctccttaatggagccgatctgaggatttcatgagcctgaaggtctacctgaccatcccggcctctattaatttagctttgaggtgggggagggggcgaggtaaggaaggcatagaaaacaatgactgtgatcccagctctcaggaagccaaggcaggcagaccacaagcctaatgacagcccaggatccatcacagagagaccctgtcaacgaagaagaggggctgatgagatggctcagcggttaagagcactgactgctcttccaaaggtcctaagttcaattcctagcaaccacatggtggctcacaaccatctgtaatgagatctgacgccctcttctggtgtgtctgaagacaactacagtgtactcacacacacacatgaaagaaagaaagaaagaaagaaagaaagaaagaaagaaagagatctaaagaaaagaaagaagggcgggaggggtggatgagaggggatgaaggaggtgtaggggattccctatgtttccaatctcaaaaggtagagtttcctgaccagttagtttctcctcccaggacctatttccagagactcaaaaccagcaccggcaatctacgagactctgataagaagtaagggctaccctggtgacttctcaccaagcttcacagagttacagagacattttgtgaaaactcgcccagttaaactgaagaacctcctccggttggtgaagttctggtacctgcaggtaagagctgggaggaggcggagcagagggtacgcacgcatctgtagcccaagcttctcttagaaattggggctggaggtgtggcttgttggcaaggtacttgcttgggatgcgtaaggtcctgggttcaatcctccgcaacagcaagagagatcgagtggggatacaagaagtgggtgagaggcaggataaacagtgagtgacctgcagggccctggaccatctaagaacccagcatccctgtcggtgagagcgtctgcatcttgtgacacctgcttgcagtgaaaccttcccccaagtgcgattaagtgctcactcagatgaggacaggctgccttcaacaagcctcagatgtgtggcattttgataacaataataaagccaccattgtgtcagactaattgggtcccatgatctaggctatacatgccaagggaccccgttttgccttccttcctgtttctttactttttttaattggttattttatttacatttcaaatgttatccccctttcccagtttcccctctggaaaccccctatcccaccccctccctccttctgctgtttctaatggcatgtatccccgccctcaccctggaggtgagaaaactgagctgagatttgaacacgagtccctgactctagaatctgtgctcgtaagcagtcccaggaaatgttcttcagtttagcatttctttcttgctatctgactaattgcaaagaactcattaaaaaaaaaaaaagtcgaaaaatgcttaaggaagaaggagagatttgcacacggtccccagataaacctgcgacagtactttggcttttctttcatgttttaccgtcatctgtattcccgcagcccatacctggatgttagataactggagttaccatgccaactcaccgcagttttggtccaccctttaaacacacacacgagctgttgtccttgagtcgatggtgagagctccgtccattcatgttgtccacacactttactttgtgtcttccagtgaactgtaagacctcccaggctacaggtatttacctcccttcccatcagggctacaaacattttcatccagccaccagccacgtactggctttaaccaggccagctctgaaaacagtaggctggagagaaggctcaacagttaagagcacccacccttgcaccagacccactttgggcagctcatagccactgtcactccagctccaagggattcagcaccctcttctggcctctacttgcattgcatgcacatagacacagacacataatttaagaacagtgatttctttctttttttctttttttgcaaattatgtgtatagtgtgtatgtatatgtatgtatctgtgtgtatctgtgtctgtgtgtaggtctcaggatggttgtctttctcaatctcctccatatatactaggacaaagtctttcagttgaccttagaccctagacttgtctgcatggctgtctaactagctagctttctctggagagcccctgattctgcctccaaagtactagaattataggcaggcatgatacccatatcattatgagacttctaaggctctttttcccacctccctggctcatatttctgtacatgaatatttctgtcattctctaatagattttacttatctggaccccccctctctcccatagaaaaatatatgtggttatttaaatattgcaatggacccataataaaaacttcaaatagcataaaaatgtataaaaatgaaggtaaacatctttgtgtgtatgcatgtgtgcatatcagtgtgaaaatagtgcatatcagtgtgaaaatagtgcatatcagtgtgaaaagctgcatccacactcataaggggaactccgattggactcagtgtgtgtgtacatgttcacatgtgtgtgcatgtgcatgcgtttatgcaggcgtgtgcttgtgtgtgtgcatgtgtgtgaggggtaaatttgcagccacctaatttttcttatatgtttaatatatacttaattccatttttaacttcttgtatatgacgtgactttggcaacacctttatttcctttcaaataactaacaagttgtccatcacatcttgaactatgcatcctttctcgctgaactaaaatattacatttgctaaaactattaaagccacaaatctaatgagaactaattctaaacagtctacgctgttatacttggcttcctttgtaagtcctgagggattgaacccaggaccttatgcaagctcgataagtaccaggctgttaagccacatccccaactcgtgactcttgtttattcttatgccagaaactgcttcttccaagtagaaggtgaggattcacttccaaagttggcctgtgacatccacatgtgcacaccctcctctctctctctctctctctttctctctctctctctctctctctctctctctctctcacacacacacacatcactgggagccatgtatggttgacatacctgtattcccaacacctggagggctgaggctgaaaaactgcaagtttcagcctagcctgggctacacaacaagattccatctaaaaaataaaactcatcaggattttaactagcatctactactgctgtttcacccagtggacacggtgtcaaactgtcatctagattctcacattcaactcacaggttagtgtgtacctctgttcttggcagagaagcactccttccccctgcccccctccccagtgcacaggatgtcacagacagcagagtactgggcaaacattgtgggaaaggaggtaagaggagcctaagaaccagcaatgggaaggatggctacaaaatagcatcttatgggcatgtcggtctcactgccaaggtgcccacagcagcatctggctgcaggagacctgtgcaagattaagctagttagtgttctagcatccatagggaaagcagagagctactggccatcagtggctgctgggagagggattttcctcagagatgtggttcccgacctgtgcctactctggaggatggccctacacccatgcacacaaggagagctccaactgaactccaagggacataaagagaaggaaaagaggactggagaaagatgggggcatgaggaggaagggtggagggacgtggctatgatcaaaatacactctatactagtatagaatttcaaatataccgcattgcctcctccattctaactggaaactgtagattgattgatactcggacggtccgcaaggaaacccccgtgccatcagcccatgtagacctaccatgtcctggctgtgtcacataaagtaacaagggaacctgaaaattcctagagcatcaatcacatgaaattcttattcactctctccctctccctctctctctctctaccccccccccccagtgcctgagacgtaaatatggaagaggtgcagtgttgccctcaaaatatgctctggagctgctgaccatctacgcctgggagatgggtacagagagcagtgatagcttcaatctggatgaagggttcgtagccgtgatggagctcctcgtaaactaccgcgacatctgcatctactggaccaagtactacaatttccaaaacgaggtcgtcaggaactttctgaagaaacagctgaagggagaccggtgagtctatcaaccacgtgacttacggctctgaggcctagaaaccctgactggagtggcttggggaaaataaagggtttcacgtactcgaaaacattcaggggtcaccaagtcaccaagtatggcctccacatcaccaaggtcttgtaagaccccaactcaatgtgtttcttagacccctagaaacgaaacccagcatggagttctttgttctttcgccttcagcctgagagaacgatgaccactgggtgacccacccaggcgcctgacccatagtgtaagggaacaagaatgtttgccaaagatagcctgtaacctttccgtgggagatgagctgtaattcactatccccattcttacgatgtttactcagcttccccattctttgtggtcttatcctcccctcactttgtggtttattctttaaaaaccctcgactgcaactgctgggggtcgatctcctctgcccctgtgtgggttatgagctcggcctcagcatgctgattcccgaataaacctcatgcgatattgcatcaagaacagtttctctcgagttattgggatgttggctcatcccgggacttgagcgaggttctccccgaaacgggggtctttcagtctctgtctgtgagtggttcaacccttccctacccagggtcttccagtcaagttccgatttatcgcttcaaggttgcaatggttctagctttctcctctagacacctagttgccagtataagattgcttatgtctagtaaactctcagagattcattttctggagcttgctgaggttatatctccaatatctccaatcctccccctctcccaccccctttccgttcaccctcttgctacagctccacttgctccggcccgcttgctccagggccctctatctccaatcctaatggaagggctttgcttgcaggggttgcttctactgggccatctgaagaatgctctctgttggccccaccttcagttttaaatgagggagttaattatggagtatactgcaagcagaaataaaggaaaagtaacacgcatcaagagagagactttgcccttagcaaaataatgtcatcaaatccagcagagacaattgagtagcctcagcaggagagagtcagcttccaccccacctactcatggcgctcacaacatcctgcagaggtcaactggggaagctgaggcagtcagctggagttcccactttttctccaaggatgtagctcccctaccctccgagtctgtacttccagctttctgttcccactgtagcatggggtaacaaaattgcccctccccgctctttggggaggggagagggggaaatgacaaggatggtaaccatggtgacaacactggcagacacaagacagccttgcaagaaaagtttgagatccacgtttggctctgaaacagtggaggctgcggaggcccgcctccagagacagctttttagtgatcttaaacatcccgtgacaatttgcccttataatgcgttttatggttccacagagacgagggcccagaggccagtatcctgactgtaaccgagatctgccattttctcccccccaggcccatcatcctagacccagctgaccccaccaacaacctgggaagaagaaagggatgggaacaggtggctgcagaagctgctttctgcctgctgcaggtctgttgcacgactgtaggccccagcgagcgctggaatgtacaggtactatgcatactatctcctggtgagaactcacgctaacactcatttagatcacgctcttcccaaggcatcccagcaagggctgttatgccttgattcctcaggaaaccggaagagctcaagaactgctaagtctgctgcaatttgcaaaagtccttttatttccagtcttgactctgatcaatggaaaaccccctacagtgtaggttgagagtgctactccccacccccacccccccagcttagcacttatatacagagcagttagaacatctttgaatgcttagggtgaaggggaggtaattcagacacgataccatttggtggggcgggactgccaggctgggagggggattaaagattgtcttttgtggggctatcaggaactagttttatggcgggcaataactgtgtgtgacctctgattacttcttttctatgatttaagcatgggacccgaatctcctttcttcaaggataggcaccaactgtacctggttatctaggagagtgctagccttggcaccactgaagctgagcttctgaggggcaatgtagaggcaagccccagacacaagccttgcagcgcttgctgtgggagaagcctagggaaaggggtggggggacttctgcaggagagtgcagaagcaagcagcctgtgcacctcagggccgtgtatttaggtatcaaagcccagtttctatctataaacacccaggcctcagttcattcatcggtaagatgggcagacaatctgccctggtctatagtcacctcagagacagagtctaataatttacctctagcttggatctagagtaatattttctcatttggtggatcatcgtcactgttttagtcagggtctctattcctgcacaaatatcatgaccaagaagcaagttggggaggaaagggtttattgggcttacacttccatgctgctgttcatcaccaaggaagtcaggactggaactcaagcaggtcaggaagcaggagctgatgcagaggtcatggagggatgttctttactggcttgcttcccctggctttctcagcctgctctcttatggaaccaagactaccagcccagaaatggtcccacccacaaggggcctttcccccttgattactaattgagaaaatgccttacagttggacctcatggaggcatctccccaactgaagctcctttctctgtgataaccccagctgtgtcaagttgacacaaaactagccagtacaatcgcttactatttaaatagtaagtgtaatatgttttttaaaaattaagtgagtcttctacagctcactgcagacagtaagtccactgtacagccttgccccataggccaccaccatcttggatctgaatgcaaatccaatgtttggattcaagaattggactgagaggtttcccgaggaagctgtttccctaacttgctaaacccacagaaatcctgtcccttggcccatttcttattttcttcgggaaaagttgtgatttgtcctcaaaagtggccccacagcaaatgaaacaaggtagacagatagagtctgaaagggacctgccatctggccatcaagtgccaatagctgagaatgccccccccccccaaaaaaaaaaaaacaaaaacaaaaaacaaaaaacaaaggaccctttgggaaacaaggaaaaatgaaaaaaaaaaaaaaaactacttatgtctcttgtctcagttagggtttctactgctgtgaacagacaccatgactacagagcagctcttaaaaaggaaaacactggggctggcttacagggtctgagaatcattccattatcgtcaggcgggaagcatggcagtgtgcaggcagacatggtgctggagaaggggctgagagtccttgatccgcaggcagcaggcagagacctggctggagcttctgagacctcaaagcccacctccataaagatacatttcttccagcaaggccacactcactcccggaagccacacccactctagcaaggccacacctcctgctagagccattccctatggtgcggggatggggagcgatttttattcaaaccaccagtctctctccatctcagaccccagtttctcagaaattccattttccagagaccagctctcagcctcttacctccctctgtgaatagcgcctccgctgagcgtgtgtggtccctctgcgtgttctcacttcacttaagttattgttcagtgggttattctgttactaaattatcttaacagacaattccctagtcgcaaaaacagatccagaagggaccacggtggagtacaagtcagagcacttctgagcatcacctgggggtgaggttgattatagcagctcaaaggaccagtgccaggattgtgctcaacaaatatgaggaaaaaaaaaaaacatttctgttacttttctgtgtggtagaaaactatttaaaccccgggccatagactatttatgttccttgttattctttactttattatattttttaaaagatttatttagccgggcggtggtggggcacgcctttaatcccagcacttgggaggcagaggcaggcggatttctgagttcaaggacagcctggtctacagagtgagttccaggacagccagggctatacagagaaaccctgtctcgaaaaacaaaacaaaacaaaacaaaacaaaaaagatttatttattatatgtaagtaaactatagatgccttcagacataccagaagagggcgtcagatcttcctatggctggttgtgagccaccatgtggttgctgggatttgaactcaggaccttcagaagagcagtcagtgttcttaagcactgagccatcttaccagatcagatattttctttatttacatttcaaatgtaattaaaccttccctggtttaccctctgaaaacctgctatcccatcctccctcctctgctgctatgagggtgttcccttcacccacccacccactcctgcctcccggccccggccccggcattcccctatttttttggttttcgttttcgttttggttttggttttggtttatttaattttttttttaattttttgttttttttttttaagacagggtttctttgcatagtcctggaactcactctgtagaccaggctggcctcaatcccaaaacctgtacctgccagcccccccacccccacacacacacacactgatatactaggatcctcctgtaagtcctgtaagtgctcggctgagggacgtctcatttaacctaccaggagaccattccagatccaagccatcttttcagcaaatatgtcagaattctgtgacgtgacatattttaatgtctagaacctaaccctgaagcagaatttttaggacacatccagacttttggcataacagtgatcgcccttgaacaaccctcctaaagccagacacaactattaaatgacatcactgtaatccccaggctcccataaggacaacgctgttgtgtacccgtgatgggtgacgattctgagtaattccacaggggaagagtatcatttagaatccatttgttgggctggagagatggctcagcagttaagagcactgactgttcttccaaatgtcccgagttcaattcccagcaacatgatggctcacaaccatctgtaatgggatctgatgccctcttctggtgcatctgaagacagcaatattatatatctatataatatatatctgtacacagatatagtgtacatagataaataaatctttaggctggagagagcagggctgaccagagcgagcaaccttcattctcagcaaccacatgatggctcacaaccatcagtacagccacagtggactcatatacataaaatttataaaatgttcgaggaacatggcgtgagttagtggtactctgagcctagggtagccaagcagctcacctgttgtctcacgccattgttcgttatgtctcccaggttgtcttggtagatgataaactatatcaggagctagaagaaagcagctttgtagacaatagacagttctttccatacacttgccctttctcttctctcccttgcagcgagcgagggatgttcaggtgagagtgaaacaaacgggaacagtggattggacactctggacaaacccctacagccccatcagaaagatgaaggcagagatcaggagggaaaagaactttggaggggaactgcgcatctccttccaggagcccggaggggagagacagctgctcagcagcaggaagaccctagcagattatgggatattctctaaggtgaacattcaagtgctggagacctttcctcctgagatcctggtctttgtgaagtatcctggtggccagagcaagcctttcaccatcgaccctgatgataccatcttagatctgaaagagaagatagaagatgctggaggcccttgtgcggaggatcaggtactactgttggatgatgaggagttggaagatgatgagagccttaaagagcttgagatcaaagactgcgacaccatcatactcattagggtaatcgactagtctggctgcttgagtccgtgcctagagtcttacgcctccctacctcatccctagactctgatgggttctttctatagcttaaatatcaacattcccttctcttgcttaaaaccctctcctatagccgggcagtggtggtgcatgcctttatctcagcacttagagaatttctgagttcaaggccagcctggtctatagagtgagttacagaacagccagagctatacggagaaaccctgtctcagataaacaaacaaacaaacaaacaaaccctctcctgtgttttccaataatacagaatgaagttcaagtttcttttcctaacagtcaattaagctgaaccctgcttagtccccgacccccaactctaaacactcaactcatactgaatgcagagtgacaccttgagtcacactgagagctgagtcacacctgtgtctcttttcctcaaacatcctttccctcagcctctgttttcatcattctgtgagtcaccgaatctcaaagagcagaccccagactgtcagattccccacctctccagaaacctcttagaatggaaaactaacccaagccagacatttcaccgagactcagcggtcctggacttaacaagcctttcacatcattctgcatgctcctgtttgaaaacaactactatctggcccatgcagcaggccaaatcatcttgtgttcccgccctctcccttcatccatgttagactcccgtggacgtaaatctatctcccttgtctaatcagactcgattgtttttaatgatttaggccttgtgctctagaatggtaccaatccagtatcaatgtatgacaagtgcttgttaaataaagaagcgaaccagagaatggctttggatcctc

SEQ ID NO: 9 - DNA, 3136 bp, Mus musculus
cactcactgaagataactagacagaaaacatcgtgagactacctcacgctgtccattgaccaggagcaatcctttgtgcctcacatctttgggactgcggtaggcatcagagaaatgtcacctcgatgggccctgcagctggagagtctgaccctggatatggccttcagctaagacacctgcacagatgatgagcagcctcggggagactagctgaggtcggtagttaggaatgtggagtgtggatgttgtgaccctcagcataagcaggaataacctagcctaaccctatcgctcgagacgccactagtaacggagacactatgcgtgattaagaatgaaataaatgcatttgccactgccagatcaccaggcagatgagattatgtggccaaatcgctgccactgaaactgctgaatcttactctgttgtggtaaacaacatcctgtgctcacaacagagccggtggggagactgcatccttaactcctgttcccttctgccttgaagaaggtaaactggatctccctgcagtttcactttcaattcttgtcaaagcttggtttctgtttccagtttctccaaggagggaggggcagagcgcttttcttctttccatgcaactctccatcccattgtctcctccagagaacgctgcaggaagcttctcccatcagagctgagcagaagctcagaggatttatctagaggacaggctggcccagctgaaatggacccgttccccgacctgtatgcgacccctggggacagtctagaccacttcctggaacacagccttcagccccagagggactggaaagaggaagggcaggatgcctgggagagaatcgagaggttctttcgggaacagtgcttccgtgatgagctgctcctggaccaagaagtcagggtgattaaggtggtgaagggaggctcctcgggaaaggggacaacgctgaaccacagatctgaccaagacatgattctgttcttaagctgcttttccagtttcgaagagcaggcgagaaaccgggaggtcgtcatcagcttcattaagaagaggctgattcattgtagcagaagcctggcctacaacatcattgtccttacccacagagagggcaaaagggcccctcgctccctcaccctaaaggttcagtcccggaagactgatgacattatttggatggatatcctcccagcttacgatgctttgggacctatttccagagactcaaaaccagcaccggcaatctacgagactctgataagaagtaagggctaccctggtgacttctcaccaagcttcacagagttacagagacattttgtgaaaactcgcccagttaaactgaagaacctcctccggttggtgaagttctggtacctgcagtgcctgagacgtaaatatggaagaggtgcagtgttgccctcaaaatatgctctggagctgctgaccatctacgcctgggagatgggtacagagagcagtgatagcttcaatctggatgaagggttcgtagccgtgatggagctcctcgtaaactaccgcgacatctgcatctactggaccaagtactacaatttccaaaacgaggtcgtcaggaactttctgaagaaacagctgaagggagaccggcccatcatcctagacccagctgaccccaccaacaacctgggaagaagaaagggatgggaacaggtggctgcagaagctgctttctgcctgctgcaggtctgttgcacgactgtaggccccagcgagcgctggaatgtacagcgagcgagggatgttcaggtgagagtgaaacaaacgggaacagtggattggacactctggacaaacccctacagccccatcagaaagatgaaggcagagatcaggagggaaaagaactttggaggggaactgcgcatctccttccaggagcccggaggggagagacagctgctcagcagcaggaagaccctagcagattatgggatattctctaaggtgaacattcaagtgctggagacctttcctcctgagatcctggtctttgtgaagtatcctggtggccagagcaagcctttcaccatcgaccctgatgataccatcttagatctgaaagagaagatagaagatgctggaggcccttgtgcggaggatcaggtactactgttggatgatgaggagttggaagatgatgagagccttaaagagcttgagatcaaagactgcgacaccatcatactcattagggtaatcgactagtctggctgcttgagtccgtgcctagagtcttacgcctccctacctcatccctagactctgatgggttctttctatagcttaaatatcaacattcccttctcttgcttaaaaccctctcctatagccgggcagtggtggtgcatgcctttatctcagcacttagagaatttctgagttcaaggccagcctggtctatagagtgagttacagaacagccagagctatacggagaaaccctgtctcagataaacaaacaaacaaacaaacaaaccctctcctgtgttttccaataatacagaatgaagttcaagtttcttttcctaacagtcaattaagctgaaccctgcttagtccccgacccccaactctaaacactcaactcatactgaatgcagagtgacaccttgagtcacactgagagctgagtcacacctgtgtctcttttcctcaaacatcctttccctcagcctctgttttcatcattctgtgagtcaccgaatctcaaagagcagaccccagactgtcagattccccacctctccagaaacctcttagaatggaaaactaacccaagccagacatttcaccgagactcagcggtcctggacttaacaagcctttcacatcattctgcatgctcctgtttgaaaacaactactatctggcccatgcagcaggccaaatcatcttgtgttcccgccctctcccttcatccatgttagactcccgtggacgtaaatctatctcccttgtctaatcagactcgattgtttttaatgatttaggccttgtgctctagaatggtaccaatccagtatcaatgtatgacaagtgcttgttaaataaagaagcgaaccagagaatggctttggatcctc

SEQ ID NO: 10 - protein, 508 aa, Mus musculus
MDPFPDLYATPGDSLDHFLEHSLQPQRDWKEEGQDAWERIERFFREQCFRDELLLDQEVRVIKVVKGGSSGKGTTLNHRSDQDMILFLSCFSSFEEQARNREVVISFIKKRLIHCSRSLAYNIIVLTHREGKRAPRSLTLKVQSRKTDDIIWMDILPAYDALGPISRDSKPAPAIYETLIRSKGYPGDFSPSFTELQRHFVKTRPVKLKNLLRLVKFWYLQCLRRKYGRGAVLPSKYALELLTIYAWEMGTESSDSFNLDEGFVAVMELLVNYRDICIYWTKYYNFQNEVVRNFLKKQLKGDRPIILDPADPTNNLGRRKGWEQVAAEAAFCLLQVCCTTVGPSERWNVQRARDVQVRVKQTGTVDWTLWTNPYSPIRKMKAEIRREKNFGGELRISFQEPGGERQLLSSRKTLADYGIFSKVNIQVLETFPPEILVFVKYPGGQSKPFTIDPDDTILDLKEKIEDAGGPCAEDQVLLLDDEELEDDESLKELEIKDCDTIILIRVID

SEQ ID NO: 11 - DNA, 21 bp, Mus musculus
aggtcggtgtgaacggatttg

SEQ ID NO: 12 - DNA, 23 bp, Mus musculus
tgtagaccatgtagttgaggtca

SEQ ID NO: 13 - DNA, 23 bp, Mus musculus
gtccggttaaatccagaagatcc

SEQ ID NO: 14 - DNA, 21 bp, Mus musculus
tagctttggcaagatgtgctg

SEQ ID NO: 15 - DNA, 23 bp, Mus musculus
cctacataaagcacctagatggc

SEQ ID NO: 16 - DNA, 22 bp, Mus musculus
atgtgatagtagatccaggcgt

SEQ ID NO: 17 - DNA, 22 bp, Mus musculus
tgatgagctactactggtcagc

SEQ ID NO: 18 - DNA, 22 bp, Mus musculus
gatctcttagcacaaggatggc

SEQ ID NO: 19 - DNA, 21 bp, Mus musculus
cagctccaagaaaggacgaac

SEQ ID NO: 20 - DNA, 21 bp, Mus musculus
ggcagtgtaactcttctgcat

SEQ ID NO: 21 - DNA, 20 bp, Mus musculus
gagactggctattgggggag

SEQ ID NO: 22 - DNA, 19 bp, Mus musculus
gaccgaaatgcttccaggg

SEQ ID NO: 23 - DNA, 23 bp, Mus musculus
catctatgaggtctttctgacgc

SEQ ID NO: 24 - DNA, 20 bp, Mus musculus
ttaggccatactcccccagc

SEQ ID NO: 25 - DNA, 21 bp, Mus musculus
ttgtgcggaggatcaggtact

SEQ ID NO: 26 - DNA, 21 bp, Mus musculus
tgatggtgtcgcagtctttga

SEQ ID NO: 27 - DNA, 21 bp, Mus musculus
acttgggtacaacattgcgag

SEQ ID NO: 28 - DNA, 22 bp, Mus musculus
gttcacaagaatctggggtgtc

SEQ ID NO: 29 - DNA, 21 bp, Mus musculus
ggagctcatctatgagaaggc

SEQ ID NO: 30 - DNA, 21 bp, Mus musculus
aagacgaaggagctgcagaac

SEQ ID NO: 31 - DNA, 20 bp, Mus musculus
ccgagctggaaagtaaagcg

SEQ ID NO: 32 - DNA, 20 bp, Mus musculus
atgggaaagacaacgaagcg

SEQ ID NO: 33 - DNA, 23 bp, Mus musculus
gcataacttgcgaaaattcagcc

SEQ ID NO: 34 - DNA, 23 bp, Mus musculus
tcagaatcctttgctcttccaga

SEQ ID NO: 35 - DNA, 22 bp, Mus musculus
agaacaatccagactagcagca

SEQ ID NO: 36 - DNA, 21 bp, Mus musculus
gggaacttcacatcacagctc

SEQ ID NO: 37 - DNA, 21 bp, Mus musculus
gatcggtgctaagttctggga

SEQ ID NO: 38 - DNA, 21 bp, Mus musculus
agggacatacttgccacctgt

SEQ ID NO: 39 - RNA, 25 nt, Artificial
  Irf7 R1
gggauccaguugauccgcauaaggu

SEQ ID NO: 40 - RNA, 25 nt, Artificial
  Irf7 R2
cgagugcuguuuggagacuggcuau

SEQ ID NO: 41 - RNA, 25 nt, Artificial
  Irf7 R3
ccaguccugcugagcucccagauca

SEQ ID NO: 42 - RNA, 25 nt, Artificial
  Isg15 R1
cagcagcacagugaugcuaguggua

SEQ ID NO: 43 - RNA, 25 nt, Artificial
  Isg15 R2
gcacagugaugcuagugguacagaa

SEQ ID NO: 44 - RNA, 25 nt, Artificial
  Isg15 R3
ugaggucuuucugacucagacugua

SEQ ID NO: 45 - RNA, 25 nt, Artificial
  Oasl2 R1
cgggaggucgucaucagcuucauua

SEQ ID NO: 46 - RNA, 25 nt, Artificial
  Oasl2 R2
gaccaagacaugauucuguucuuaa

SEQ ID NO: 47 - RNA, 25 nt, Artificial
  Oasl2 R3
ccugcagugccugagacguaaauau

SEQ ID NO: 48 - RNA, 25 nt, Artificial
  Rig-I R1
gggaucccagcaaugagaauccuaa

SEQ ID NO: 49 - RNA, 25 nt, Artificial
  Rig-I R2
gcagaacuggaacaggucguuuaua

SEQ ID NO: 50 - RNA, 25 nt, Artificial
  Rig-I R3
ggaagccaugcaacauaucuguaaa

(19) United States
(12) Patent Application Publication
(10) Pub. No.: US 2011/0104138 A1
(43) Pub. Date: May 5, 2011

(54) USE OF THE INNATE IMMUNITY GENE OASL FOR PREVENTING OR TREATING INFECTION WITH NEGATIVE STRAND RNA VIRUSES

(75) Inventors: Jean-Jacques Raoul Panthier, Paris (FR); Tania Zaverucha Do Valle, Paris (FR); Agnes Marie Monique Billecocq, Paris (FR); Michele Bouloy, Paris (FR); Xavier Bernard Philippe Montagutelli, Hermeray (FR)

(73) Assignee: INSTITUT PASTEUR, Paris (FR)

(21) Appl. No.: 12/611,746

(22) Filed: Nov. 3, 2009

(51) Int. Cl. A61K 38/45; A61K 31/7088; C12N 9/10

(52) U.S. Cl. 424/94.5; 514/44 R; 435/193

(57) ABSTRACT

A method to treat a disease, disorder or condition caused by a negative-sense single-strand RNA virus in an individual in need, comprising at least the step of administering to said individual in need, an isolated 2′-5′-oligoadenylate synthetase like protein or an isolated polynucleotide encoding said 2′-5′-oligoadenylate synthetase like protein.

### BACKGROUND OF THE INVENTION

1. Field of the Invention

The invention relates to the medium form of 2′,5′-OligoAdenylate Synthetase Like (OASL) as a medicament for preventing infection with or treating an infection of negative-sense single strand RNA viruses such as Rift Valley Fever. The invention also relates to the use of the 2′,5′-OligoAdenylate Synthetase Like (OASL) gene as a marker for genetic susceptibility to negative-sense RNA viruses and in particular Rift Valley Fever Virus.

2. Description of the Related Art

Negative-strand RNA viruses also known as antisense-strand RNA viruses, are viruses whose genome consists of at least one strand of RNA which does not encode mRNA. According to the Baltimore classification, Negative-strand RNA viruses are contained within Group V and comprise the Order Mononegavirales which comprises the Families Bornaviridae, Filoviridae, Paramyxoviridae and Rhabdoviridae; as well as a number of unassigned families Arenaviridae, Bunyaviridae, Orthomyxoviridae and Genera Deltavirus, Nyavirus, Ophiovirus, Tenuivirus and Varicosavirus.

Several negative-strand RNA viruses cause serious diseases such as influenza, measles and rabies. Another important disease caused by a negative-strand RNA virus is Rift Valley Fever (RVF). RVF is an arthropod-borne disease caused by a virus of the Bunyaviridae family, genus Phlebovirus. Originally present in Eastern and Southern Africa, RVF virus (RVFV) has spread in recent years to Western Africa, Madagascar, and even outside Africa, in Saudi Arabia and Yemen [1]. In natural ecosystems, RVF affects mainly sheep, cattle, goats, and humans, but other mammals, such as camels, buffaloes, horses and dogs, may also present the disease. Infection of horses is often unapparent or subclinical, but carnivores—dogs and cats—exhibit viraemia [2].

RVFV is transmitted mostly by mosquitoes in the genera Aedes and Culex, though other arthropods may play a role in the spread of the virus [3]. RVF outbreaks represent a threat for humans in endemic areas, where people may be infected by mosquitoes, direct contact with animals or even raw milk [2] [3]. Outbreaks which may last several months occur during periods with heavy rainfall; they inflict severe economical losses, especially upon trade activities [3].

The RVFV has a tripartite single-stranded RNA (ssRNA) genome, consisting of large (L), medium (M) and small (S) segments. The L and M segments are of negative polarity, while the S segment uses an ambisense strategy. The S segment encodes the N nucleocapsid and the NSs non-structural protein in antisense and sense orientation, respectively [4]. NSs is an important factor of RVFV virulence. Indeed, the deletion of 69% of the NSs open reading frame in RFV virus Clone 13, an isolate from the Central African Republic, is responsible for its avirulence in mice [5]. NSs protein acts through several independent mechanisms. First, NSs induces the specific degradation of the double-stranded RNA (dsRNA)-dependent protein kinase PKR/EIF2AK2. In the absence of NSs, PKR is activated by dsRNA generated during viral replication and 5′-triphosphated ssRNA and phosphorylates the α subunit of the translation initiation factor eIF2 leading to inhibition of viral protein synthesis [6,7].

In the presence of NSs, PKR is downregulated thus facilitating efficient viral translation. Second, NSs sequesters p44, a subunit of the general transcription factor II H (TFIIH). This interaction of NSs with p44 affects the assembly of TFIIH complex and thus inhibits cellular transcription [8]. Third, NSs interacts with Sin3A Associated Protein 30 (SAP30), a subunit of histone deacetylase complex, and maintains the promoter of interferon-β (Ifnb1) gene in a transcriptionally silent state, thus blocking production of IFN-β [9].

In domesticated animals, RVF usually causes miscarriage in pregnant females and it is often fatal for the newborn. In humans, the disease leads to a wide variety of clinical manifestations that range from a febrile influenza-like illness to retinitis, encephalitis and hepatitis with fatal hemorrhagic fever [3]. Age is an important determinant of the virulence of RVF, but it cannot account for the various outcomes of RVFV infection, in animals nor in humans. Genetic determinants therefore seem to play an essential role in modulating infectious disease outcomes. A wide variation in susceptibility to RVF is observed in different livestock breeds, from unapparent or moderate febrile reactions to high fevers, severe prostration and death in the most susceptible animals [2]. It was recognized that breeds indigenous to the tropical or subtropical African zones are resistant, while European or imported genotypes, exotic to the continent, are highly susceptible [2,3]. On the other hand, the West African dwarf sheep breed is highly susceptible to experimental RVFV infection despite its indigenous origin [10]. Likewise, indigenous livestock were reported to be severely affected by RFV during outbreaks, as observed during the Egyptian outbreak in 1977-78 [11]. Additional epidemiological inquiries will be needed to clarify this issue.

Innate antiviral mechanisms mediated by Type-I interferons (IFN-α/β) are potentially the most important pathways of host cell defense in limiting viral replication. IFN-α/β are able to trigger the activation of a specific signal transduction pathway leading to the induction of IFN-stimulated genes (ISGs) that are responsible for the establishment of an antiviral state. The ISGs believed to affect RNA virus replication in single cells are the RNA-specific Adenosine Desaminasc (ADAR), the proteins of the myxovirus resistance (Mx) family, the double-stranded RNA-dependent protein kinase (PKR), and the 2′,5′-oligoadenylate synthetase (2′,5′-OAS or OAS) family associated to endoribonuclease RNase L.

Human OAS is a family of enzymes encoded by three closely linked genes on chromosome 12q24.2, with the following order: small (OAS1, p40/46), medium (OAS2, p69/71), and large (OAS3, p100) OAS isoforms [62-66]. Each OAS gene consists of a conserved OAS unit composed of five translated exons (exons A-E). OAS1 has one unit, whereas OAS2 and OAS3 have two and three units, respectively, and all three genes encode active 2′,5′-Oligoadenylate Synthetase. Another gene, OASL (OAS-Like) encodes a single-unit of OAS-like protein, which however, lacks 2′-5′ synthetase activity [68-69]. Within each size class, multiple members arise as a result of alternate splicing of the primary transcript. The OAS proteins share a conserved unit/domain of about 350 amino acids (OAS unit); OAS1 (p40/p46), OAS2 (p69/71) and OAS3 (p100) contains one, two and three tandem copies of the OAS unit, respectively.

Each OAS protein accumulates in different cellular locations, require different amounts of dsRNA to be activated, and catalyse the formation of differently sized 2-5A products. OAS 1 functions as a tetramer, OAS2 is only active as a dimer and OAS3 has been observed only as a monomer. In addition, the large form of human OAS is presumably not involved in RNase L activation [70].

The first direct evidence for the involvement of OAS family in the antiviral effect exhibited by IFN was provided by transfection of 2′,5′-oligoadenylate synthetase (OAS) cDNA into cells. Overexpression of OAS1 or OAS2 leads to resistance of cells to picornavirus replication [71]. The importance of OAS1 for clearing West Nile Fever virus (WNV) infection in vivo was also supported by the finding that murine Oas1b, the orthologous gene of human OAS1, may play a key role into the susceptibility/resistance phenotype of mice to WNV-induced encephalitis [72-75]. Analysis of the OAS genetic polymorphism in human demonstrated that genetic markers in OAS genes were the most strongly associated with enzyme activity. Given that OAS1 is an excellent candidate for a human gene that influences host susceptibility to viral infection [75], genetic variations in human OAS1 as well as OASL genes were associated to the risk of viral encephalitis, type 1 diabetes mellitus (DM), Hepatitis C virus (HCV) related disease and other virus infection. With a particular emphasis on HCV disease, a series of OAS1 genotypes linked with the outcome of HCV infection has been reported [77-78].

### BRIEF SUMMARY OF THE INVENTION

For the first time, the inventors demonstrate a role for OASL in the endogenous antiviral pathway against negative-sense ssRNA viruses such as RVFV. In particular using a series of susceptible and resistant mouse models, microarray analysis and siRNA mediated gene suppresion the inventors have shown that variations in the OASL2 gene (2′,5′-OligoAdenylate Synthetase-Like 2, the mouse orthologue of OASL) are strongly linked to variations in the ways in which these mice models are susceptible/resistant to RVFV.

These findings are useful for the development of OASL-based prophylaxis and therapy against negative-sense ssRNA viruses of major medical importance including RVFV, influenza and rabies. They are also useful for the development of new OASL-based molecular tools for the prediction of human susceptibility to the infection with negative-sense ssRNA viruses, of major medical importance, and in particular for the prediction of susceptibility to Rift Valley Fever.

A subject of the invention is an isolated 2′,5′-oligoadenylate synthetase like protein or an isolated polynucleotide encoding said 2′-5′-oligoadenylate synthetase like protein, as a medicament.

### BRIEF DESCRIPTION OF THE DRAWINGS

For a better understanding of the invention and to show how the same may be carried into effect, there will now be shown by way of example only, specific embodiments, methods and processes according to the present invention with reference to the accompanying drawings in which:

FIG. 1—Survival analysis of inbred strains of mice. Fifteen males of each inbred strain of mice were inoculated with 10² plaque-forming unit (PFU) of RVFV by intraperitoneally injection and followed for mortality for 14 days. Statistical differences were evaluated using the Kaplan-Meier test. Asterisks indicate values that are statistically significant (*, p<0.05; **, p<0.01; ***, p<0.001).

FIG. 2—Virological analysis of BALB/cByJ and MBT/Pas cells. Viral production by BALB/cByJ and MBT/Pas mouse embryonic fibroblasts (MEFs) at 15 and 20 hours after infection with RVFV at MOI of 1, 5 and 10. Statistical analyses were performed by Student's t test on log₁₀ transformed data (**, p<0.01; ***, p<0.001).

FIG. 3—Microarray analysis of BALB/cByJ and MBT/Pas cells at 9 h following RFV virus infection. (A) Total number of genes whose expression was differentially modulated in RVFV-infected BALB/cByJ and MBT/Pas cells compared to mock-infected cultures. Numbers for up and downregulated genes are in red and green respectively. (B) Venn diagram of the number of genes enriched (red ↑) or impoverished (green ↓) in BALB/cByJ and MBT/Pas MEFs and their overlap. (C) Enrichment of functions by upregulated (red) or downregulated (green) genes in BALB/cByJ MEFs. (D) The enrichment of identical functions in MBT/Pas cells.

FIG. 4—Expression profiles of RVFV-responsive genes 9 h post-infection. (A, B) Heat maps showing genes whose expression was modulated by infection in BALB/cByJ (A) and MBT/Pas (B) cells. (C) The heat map shows 29 genes related to the IFN innate immune response that were upregulated post-infection in BALB/cByJ cells. (D) The expression modulation of these 29 genes in mock- and RVFV-infected MBT/Pas cells. Green and red squares indicate decreased and increase levels of expression, respectively. Black bars indicate no change in expression level. The color scale indicates the change magnitude. Values are in log₂.

FIG. 5—Genes induced by RVFV infection in BALB/cByJ and MBT/Pas cells 9 h post-infection. The IFN-α/β gene induction occurs in two steps. The left panel shows the early signaling events following virus infection. Viral components are sensed by cytoplasmic pathogen recognition receptors (PRRs), as PKR, MDA5, RIG-I and DAI. These sensors trigger cascades which activate NFκB and IRF3. These proteins enter the nucleus and stimulate the transcription of Ifnb1 and interferon-stimulated genes (ISGs), such as Isg15 and Ifit1. The produced IFN-β binds the type I IFN receptor (IFNAR) and activates JAK/STAT pathway (late signalling events, on right panel). Phosphorylated STAT1 and STAT2 bind IRF9 to form ISGF3. ISGF3 enters the nucleus and stimulates ISGs transcription. Genes induced at this stage include Oas1a, Oasl1 and Oasl2 genes, Isg20, Ifi27, cytoplasmic PRRs-encoding genes and Irf7. IRF7, together with IRF3, activates Ifna and Ifnb1 genes thus creating a positive feedback loop. The IFN-α/β gene induction mechanism is stimulated by RVFV despite the inhibition of Ifnb1 gene by the viral NSs protein (shown in purple oval). Red squares indicate genes upregulated in BALB/cByJ cells, but not in MBT/Pas cells. Red-black checkerboard squares indicate genes upregulated in both BALB/cByJ and MBT/Pas cells. Black squares indicate genes whose expression was not changed by the infection. White squares indicate genes for which there was no information in the microarray chip.

FIG. 6—Induction kinetics of immune response genes in BALB/cByJ and MBT/Pas cells by the RVFV. Genes were classified in three groups according to their induction profile following infection. The first one encompasses Ifit3 (A), Ifna4 (B) and Ifnb1 (C) genes that exhibited higher expression in MBT/Pas cells late after infection. Ifit1 (D), Rig-I (E) and Stat2 (F) belong to the second group of genes whose expression in MBT/Pas cells was delayed. Finally Irf7 (G), Isg15 (H) and Oasl2 (I) genes, of the last group, were characterized by absence or very weak induction in MBT/Pas cells even at late times. Levels are expressed in relative expression in comparison to a reference gene (Tbp). Statistical analysis was performed by Student's t test on log₁₀ transformed data. (*, p<0.05; **, p<0.01; ***, p<0.001).

FIG. 7—Effect of NSs viral protein on the expression of Ifnb1 and Ifna4 genes. Quantification of Ifnb1 (A) and Ifna4 (B) mRNA in BALB/cByJ MEFs infected with the virulent ZH548 (black triangle) or attenuated rec-ZHΔNSs (white triangle) strain of Rift Valley fever virus. Statistical analysis was performed by Student's t test on log₁₀ transformed data. (*, p<0.05; **, p<0.01; ***, p<0.001).

FIG. 8—Effects of siRNA-mediated downregulation of Irf7, Isg15, Oasl2 and Rig-I genes on viral production. qRT-PCR analysis showed the inhibition of Irf7 (A), Isg15 (B), Oasl2 (C) and Rig-I (D) gene expression after transfection with either specific siRNA (RNAi-1, 2 or 3) or scramble siRNA (control, ctrl). mRNA levels are presented relative to the gene expression in cells transfected with the scramble siRNA. BALB/cByJ MEFs were transfected with the siRNAs and, 24 h later, infected with the rec-ZHΔNSs strain of Rift Valley fever virus. Total RNAs were extracted 6 h post-infection. Mock-infected MEFs were included as a control (non-infected, ni). (E) The most efficient siRNA, namely R-2, R-1, R-2 and R-3 for Irf7, Isg15, Oasl2 and Rig-I respectively, was transfected in BALB/cByJ MEFs onto twelve 35 mm plates. Twenty four hours later, the transfected MEFs were infected with the virulent ZH548 strain of Rift Valley fever virus. The numbers of viral particles in the supernatant, displayed in log₁₀ per 10⁶ cells, were measured 24 h later. Statistical analysis was performed by Student's t test on log₁₀ transformed data, always in comparison to the control (*, p<0.05; **, p<0.01; ***, p<0.001).

### DETAILED DESCRIPTION OF THE INVENTION

### Definitions

A “polynucleotide” refers to a genomic DNA fragment, a cDNA fragment or an RNA molecule. The polynucleotide may be isolated or purified.

An “isolated” agent, including a polynucleotide or protein product, is one which has been identified and separated and/or recovered from a component of its natural environment.
The terms “2-5A synthetase L”, “2′-5′-oligoadenylate synthetase L”, “(2-5′)oligo(A) synthetase L”, “OASL”, refer to a protein which is encoded by the OASL gene of a mammal and which may have 2′-5′-oligoadenylate synthetase activity, and to the derived variants, including natural variants resulting from polymorphism in the OASL gene and artificial variants resulting from mutation (insertion, deletion, substitution) of one or more nucleotides in the OASL gene/open reading frame (ORF) sequences, providing that the variant is capable of inhibiting negative-sense single-stranded RNA virus replication. The OASL gene and the deduced OASL ORF and amino acid sequence of various mammals are available in sequence databases and other OASL gene/ORF sequences may be determined by standard cloning and sequencing techniques which are known by one skilled in the art.
The term “human OASL gene” is a 18686 bp sequence (SEQ ID NO: 1) corresponding to positions 121458095 to 121476780 on GenBank sequence accession number NC_{—}000012.11. The human OASL gene is located on chromosome 12 (12q24.2). In the event of ambiguity between different versions of a sequence identified by accession number or otherwise identified sequence disclosed or described herein, reference is made to the version most contemporaneous with the filing date of this application.
A “human OASL open reading frame (ORF)” is any one of SEQ ID NO: 2 to 3 which correspond to the two known isoforms of OASL (NM_{—}003733.2, NM_{—}198213.1).
A “human OASL protein” is any one of SEQ ID NO: 5 to 6 which correspond to the three known isoforms of OASL (NP_{—}003724.1, NP_{—}937856.1).
“OASL activity” refers to negative-sense single-stranded RNA virus replication inhibition activity.

The 2′,5′-oligoadenylate synthetase activity of the OASL protein of the invention may also be assayed where such activity is known, for instance the mouse OASL2 gene product has this activity whereas the human OASL gene product does not. This activity may be assayed by chromatographic or electrophoretic methods to determine the end-point amounts of oligoadenylates formed [79-85], which are incorporated by reference.
A “mouse 2′-5′-oligoadenylate synthetase-like 2” is the mouse orthologue of human 2′-5′-oligoadenylate synthetase like. The mouse 2′-5′-oligoadenylate synthetase-like 2 gene is a 15312 bp sequence (SEQ ID NO: 8) corresponding to positions 115346943 to 115362254 on Genbank sequence accession number NC_{—}000071.5. The mouse, OASL2 gene is located on mouse chromosome 5 (5F). One known transcript of the OASL2 gene has been characterised, the nucleotide sequence of which SEQ ID NO: 9 (Consensus CDS CCDS39226.1) and the peptide sequence SEQ ID NO: 10 (NP_{—}035984) are provided.
“inhibition of negative-sense ssRNA virus replication by the OASL protein of the invention” refers to the partial or total reduction of virus growth (virus multiplication) when exogenous OASL protein (not encoded by the genome of the cells, a recombinant OASL protein, for example) is present in the virus-infected cells. This inhibition may be determined by infecting an appropriate recombinant cell line expressing the OASL protein with a positive-sense single-stranded RNA virus. Non-recombinant cells of the same type infected with the virus are used as control. Then, progeny virus production in the supernatant of the virus-infected cells may be measured by any well-known virus titration assay. Alternatively, viral proteins production may be analyzed by Western-Blot or Immunolabeling of viral antigens or viral genomic and subgenomic RNAs production may be analyzed by Northern-Blot or RT-PCR.
“negative-sense ssRNA virus” refers to a virus that has negative-sense single-stranded ribonucleic acid (ssRNA) as its genetic material and does not replicate using a DNA intermediate. Negative-sense ssRNA viruses belong to Group V of the Baltimore classification system of classifying viruses.
“identity” with respect to both amino acid sequences and nucleic acid sequences, refers to a measure of the degree of identity of two sequences based upon alignment of the sequences which maximizes identity between aligned amino acid residues or nucleotides, an which is a function of the number of identical residues or nucleotides, the number of total residues (up to 514 residues in the case of the present invention) or nucleotides (up to 1584) nucleotides in the case of the present invention), and the presence and length or gaps in the sequence alignment. Various alignment algorithms and/or computer programs are available for determining sequence identity using standard parameters, including FASTA, or BLAST which are available as a part of the GCG sequence analysis package (University of Wisconsin, Madison, Wis.), and can be used with, e.g., default settings. Amino acid and nucleic acid sequence variants, such as OASL genes or proteins, as described herein may have at least 60%, 70%, 80%, 85%, 90%, 95%, 98% or 99% identity to an other sequence disclosed herein. For example, a variant of human OASL gene of SEQ ID NO: 1 may be 90% identical or similar to SEQ ID NO: 1 and may encode a protein having at least one function of the human OASL protein encoded by SEQ ID NO: 1.
“similarity” refers to a measure of the degree of similarity of two amino acid sequences based upon alignment of the sequences which maximizes similarity between aligned amino acid residues, and which is a function of the number of identical or similar residues, the number of total residues (up to 514 residues in the case of the present invention), and the presence and length or gaps in the sequence alignment. Various alignment algorithms and/or computer programs are available for determining sequence similarity using standard parameters, including FASTA, or BLAST which are available as a part of the GCG sequence analysis package (University of Wisconsin, Madison, Wis.), and can be used with, e.g., default settings. Similar residues refer to residues having comparable chemical properties, including size, charge (neutral, basic, acidic), and/or hydrophilicity/hydrophobicity. Amino acid and nucleic acid sequence variants as described herein may have at least 60%, 70%, 80%, 85%, 90%, 95%, 98% or 99% similarity to another sequence disclosed herein. Such similarity may be determined by an algorithm, such as those described by Current Protocols in Molecular Biology, vol. 4, chapter 19 (1987-2009) or by using known software or computer programs such as the BestFit or Gap pairwise comparison programs (GCG Wisconsin Package, Genetics Computer Group, 575 Science Drive, Madison, Wis. 53711). BestFit uses the local homology algorithm of Smith and Waterman, Advances in Applied Mathematics 2: 482-489 (1981), to find the best segment of identity or similarity between two sequences. Gap performs global alignments: all of one sequence with all of another similar sequence using the method of Needleman and Wunsch, J. Mol. Biol. 48:443-453 (1970). When using a sequence alignment program such as BestFit, to determine the degree of sequence homology, similarity or identity, the default setting may be used, or an appropriate scoring matrix may be selected to optimize identity, similarity or homology scores. Similarly, when using a program such as BestFit to determine sequence identity, similarity or homology between two different amino acid sequences, the default settings may be used, or an appropriate scoring matrix, such as blosum45 or blosum80, may be selected to optimize identity, similarity or homology scores.
The terms “individual” or “subject” includes mammals, as well as other vertebrates (e.g., birds, fish and reptiles). The terms “mammal” and “mammalian”, as used herein, refer to any vertebrate animal, including monotremes, marsupials and placental, that suckle their young and either give birth to living young (eutharian or placental mammals) or are egg-laying (metatharian or nonplacental mammals). Examples of mammalian species include humans and other primates (e.g., monkeys, chimpanzees), rodents (e.g., rats, mice, guinea pigs) and others such as for example: cows, pigs and horses.

An “effective amount” refers to that amount of a therapeutic agent sufficient to reduce the severity of or treat a condition, disorder or disease, to enhance the therapeutic efficacy of another therapy of the condition, disorder or disease, or to prevent the recurrence or prevent an increase in severity of the condition, disorder or disease or at least one of its symptoms. An effective amount may refer to the amount of therapeutic agent sufficient to delay or minimize the onset of disease or to an amount of the therapeutic agent that provides a therapeutic benefit in the treatment or management of a disease. An effective amount with respect to a therapeutic agent of the invention means that amount of therapeutic agent alone, or in combination with other therapies, that provides a therapeutic benefit in the treatment or management of a disease, e.g., sufficient to enhance the therapeutic efficacy of a therapeutic agent or drug sufficient to treat or manage a disease. Used in connection with an amount of protein or polynucleotide of the invention, the term can encompass an amount that improves overall therapy, reduces or avoids unwanted effects, or additively enhances the therapeutic efficacy of, or synergizes with, another therapeutic agent.
by “mutation” is intended the substitution, deletion, insertion of up to one, two, three, four, five, six, ten, twenty, fifty or more nucleotides/amino acids in a polynucleotide (cDNA, gene) or a polypeptide sequence. The mutation can affect the coding sequence of a gene or its regulatory sequence. It may also affect the structure of the genomic sequence or the structure/stability of the encoded mRNA.

The invention encompasses modified OASL protein including one or more modifications selected from the group consisting of: the mutation (insertion, deletion, substitution) of one or more amino acids in the OASL amino acid sequence, the addition of an amino acid fusion moiety, the substitution of amino acid residues by non-natural amino acids (D-amino-acids or non-amino acid analogs), the modification of the peptide bond, the cyclization, the addition of chemical groups to the side chains (lipids, oligo- or -polysaccharides), and the coupling to an appropriate carrier. These modifications which are introduced by procedures well-known in the art, result in a modified OASL protein which is able to inhibit negative-sense single-stranded RNA virus replication activities.

According to a preferred embodiment of the invention, the 2′-5′-oligoadenylate synthetase Like (OASL) is human 2′-5′-oligoadenylate synthetase like.

According to another embodiment of the present invention, the 2′-5′-oligoadenylate synthetase like (OASL) is mouse 2′-5′-oligoadenylate synthetase-like 2.

According to another preferred embodiment of the invention, said OASL protein has at least 70%, 80%, 90% or 95% amino acid sequence identity or 80%, 90%, or 95% amino acid sequence similarity, preferably at least 80% amino acid sequence identity or 90% amino acid sequence similarity to residues 1 to 514 of SEQ ID NO: 5, or residues 1 to 255 of SEQ ID NO: 6 or residues 1 to 508 of SEQ ID NO: 10.

According to another embodiment of the invention, there is provided a polynucleotide coding for a protein as defined above.

In particular the polynucleotide preferably comprises or consists of a nucleotide sequence selected in the group consisting of: SEQ ID NO: 2 and SEQ ID NO: 3 which encode the protein of SEQ ID NO: 5 and SEQ ID NO: 6 respectively.

According to another preferred embodiment of the invention, said polynucleotide is inserted in an expression vector.

A vector refers to a nucleic acid molecule capable of transporting another nucleic acid to which it has been linked. A vector which can be used in the present invention includes, but is not limited to, a viral vector, a plasmid, a RNA vector or a linear or circular DNA or RNA molecule which may consists of a chromosomal, non chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available.

Viral vectors include retrovirus, adenovirus, parvovirus (e.g. adenoassociated viruses or AAVs), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, spumavinis (Coffin, J. M., Retroviridae: The viruses and their replication, In Fundamental Virology, Third Edition, B. N. Fields, et al., Eds., Lippincott-Raven Publishers, Philadelphia, 1996).

Preferably said vectors are expression vectors, wherein the sequence encoding the OASL protein of the invention is placed under control of appropriate transcriptional and translational control elements to permit production or synthesis of said protein. Therefore, said polynucleotide is comprised in an expression cassette. More particularly, the vector comprises a replication origin, a promoter operatively linked to said encoding polynucleotide, a ribosome-binding site, an RNA-splicing site (when genomic DNA is used), a polyadenylation site and a transcription termination site. It also can comprise an enhancer. Selection of the promoter will depend upon the cell in which the polypeptide is expressed. Suitable promoters include tissue specific and/or inducible promoters. Examples of inducible promoters are: eukaryotic metallothionine promoter which is induced by increased levels of heavy metals, heat shock promoter which is induced by increased temperature. Examples of tissue specific promoters are skeletal muscle creatine kinase, prostate-specific antigen (PSA), α-antitrypsin protease, human surfactant (SP) A and B proteins and β-casein. Vectors can comprise selectable markers, for example: neomycin phosphotransferase, histidinol dehydrogenase, dihydrofolate reductase, hygromycin phosphotransferase, herpes simplex virus thymidine kinase, adenosine deaminase, glutamine synthetase, and hypoxanthine-guanine phosphoribosyl transferase for eukaryotic cell culture; TRP1, URA3 and LEU2 for S. cerevisiae; tetracycline, rifampicin or ampicillin resistance in E. coli.

The choice of the vector depends on their use (stable or transient expression) or and on the host cell; viral vectors and “naked” nucleic acid vectors are preferred vectors for expression in mammal cells (human and animal). Use may be made, inter alia, of viral vectors such as adenoviruses, retroviruses, lentiviruses and AAVs, into which the sequence of interest has been inserted beforehand.

In particular the OASL protein or a nucleic acid encoding a OASL protein according to the present invention is useful in preventing or treating an infection caused by virus selected from the group: Borna disease virus, Ebola virus, Marburg virus, Measles virus, Mumps virus, Nipah virus, Hendra virus, Rabies virus, Lassa virus, Dugbe virus, Hantavirus, Crimean-Congo hemorrhagic fever, Influenza virus, Rift Valley Fever Virus.

The subject-matter of the present invention is also a pharmaceutical composition characterized in that it comprises at least one OASL protein or one polynucleotide encoding an OASL protein, preferably inserted in an expression vector, as defined above, and at least one acceptable vehicle, carrier, additive and/or immunostimulating agent.

Any suitable carrier known to those of ordinary skill in the art may be employed in the pharmaceutical composition of the present invention, the type of carrier varying depending on the mode of administration. For parenteral administration, such as subcutaneous injection, the carrier preferably comprises water, saline buffer, lactose, mannitol, glutamate, a fat or a wax and the injectable pharmaceutical composition is preferably an isotonic solution (around 300-320 mosmoles). For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g. polylactic galactide) may also be employed as carriers for the pharmaceutical compositions of this invention. Suitable biodegradable microspheres are disclosed, for example in U.S. Pat. Nos. 4,897,268 and 5,075,109, both incorporated by reference. The additive may be chosen among antiaggregating agents, antioxidants, dyes, flavor enhancers, or smoothing, assembling or isolating agents, and in general among any excipient conventionally used in the pharmaceutical industry. Any of the variety of immunostimulating agent may be employed in the compositions of the present invention to enhance the immune response.

The pharmaceutical composition may be in a form suitable for oral administration. For example, the composition is in the form of tablets, ordinary capsules, gelatine capsules or syrup for oral administration. These gelatine capsules, ordinary capsules and tablet forms can contain excipients conventionally used in pharmaceutical formulation, such as adjuvants or binders like starches, gums and gelatine, adjuvants like calcium phosphate, disintegrating agents like cornstarch or algenic acids, a lubricant like magnesium stearate, sweeteners or flavourings. Solutions or suspensions can be prepared in aqueous or non-aqueous media by the addition of pharmacologically compatible solvents. These include glycols, polyglycols, propylene glycols, polyglycol ether, DMSO and ethanol.

The OASL protein or the polynucleotide encoding an OASL protein (isolated or inserted in a vector) are introduced into cells, in vitro, ex vivo or in vivo, by any convenient means well-known to those in the art, which are appropriate for the particular cell type, alone or in association with at least either an appropriate vehicle and/or carrier. For example, the OASL protein/polynucleotide may be associated with a substance capable of providing protection for said sequences in the organism or allowing it to cross the host-cell membrane. The OASL protein may be advantageously associated with liposomes, polyethyleneimine (PEI), and/or membrane translocating peptides [90-93], which are incorporated by reference; in the latter case, the sequence of the OASL protein is fused with the sequence of a membrane translocating peptide (fusion protein). Polynucleotide encoding OASL (isolated or inserted in a vector), may be introduced into a cell by a variety of methods (e.g., injection, direct uptake, projectile bombardment, liposomes, electroporation). OASL protein can be stably or transiently expressed into cells using appropriate expression vectors as defined above.

In one embodiment of the present invention, the OASL protein/polynucleotide is substantially non-immunogenic, i.e., engenders little or no adverse immunological response. A variety of methods for ameliorating or eliminating deleterious immunological reactions of this sort can be used in accordance with the invention. In a preferred embodiment, the OASL protein is substantially free of N-formyl methionine. Another way to avoid unwanted immunological reactions is to conjugate protein/polynucleotide to polyethylene glycol (“PEG”) or polypropylene glycol (“PPG”) (preferably of 500 to 20,000 daltons average molecular weight (MW)).

The subject-matter of the present invention is also products containing at least an OASL protein or a polynucleotide encoding an OASL protein, preferably inserted in an expression vector, as defined above and a second product which is different from the first one, wherein the second product is selected from the group consisting of: antiviral, anti-inflammatory and immunomodulatory drugs, as a combined preparation for simultaneous, separate or sequential use in the prevention or the treatment of a negative-sense single-stranded RNA virus infection.

The subject-matter of the present invention is also a method for preventing or curing a negative-sense single-stranded RNA virus infection in an individual in need thereof, said method comprising the step of administering to said individual a composition as defined above, by any means.

In general, the composition may be administered by parenteral injection (e.g., intradermal, intramuscular, intravenous or subcutaneous), intranasally (e.g. by aspiration or nebulization), orally, sublingually, or topically, through the skin or through the rectum.

The amount of OASL (protein/polypeptide) present in the composition of the present invention is a therapeutically effective amount. A therapeutically effective amount of OASL (protein/polypeptide) is that amount necessary so that OASL protein performs its role of inhibiting positive-sense single-stranded RNA virus replication without causing, overly negative effects in the subject to which the composition is administered. The exact amount of OASL (protein/polypeptide) to be used and the composition to be administered will vary according to factors such as the positive-sense single-stranded RNA virus species and the individual species (human, animal) being treated, the mode of administration, the frequency of administration as well as the other ingredients in the composition.

Preferably, the composition is composed of from about 10 μg to about 10 mg and more preferably from about 100 μg to about 1 mg, of OASL (protein/polypeptide). By “about”, it is meant that the value of said quantity (μg or mg) of OASL can vary within a certain range depending on the margin of error of the method used to evaluate such quantity.

For instance, during an oral administration of the composition of the invention, individual to be treated could be subjected to a 1 dose schedule of from about 10 μg to about 10 mg of OASL (protein/polypeptide) per day during 3 consecutive days. The treatment may be repeated once one week later.

For parenteral administration, such as subcutaneous injection, the individual to be treated could be subjected to a 1 dose of from about 10 μg to about 10 mg and more preferably from about 100 μg to about 1 mg, of OAS3 (protein/polypeptide). The treatment may be repeated once one week later.

A subject of the invention is also a method in vitro for evaluating the susceptibility of an individual to an infection with a negative-sense single-stranded RNA virus as defined above, comprising: the detection of a polymorphism in the OASL gene in a nucleic acid sample obtained from said individual and/or the detection of the level of expression of OASL mRNA or protein.

The nucleic acid sample may be genomic DNA, total mRNA or cDNA.

The polymorphism is detected by any method known in the art that allows the detection of mutation in nucleic acid sequences as those described for example In Current Protocols in Human Genetics, 2008, John Wiley & Sons, Inc. which is incorporated by reference. Examples of genotyping assays include with no limitation: RAPD, RFLP, AFLP, sequence specific oligonucicotide hybridization, SnapShot PCR, Ligase detection reaction, PCR and Maldi-TOF, Pyrosequencing.

In particular the method for evaluating the susceptibility of an individual to an infection with a negative-sense single-stranded RNA virus comprises measuring the level of OASL mRNA and/or OASL protein in a sample from an individual and comparing this to previously measured levels of OASL mRNA and/or OASL protein in a range of individuals whose susceptibility to the negative-sense single-stranded RNA virus has been determined.

According to a further aspect of the present invention there is provided a model system to study the effects of Rift Valley Fever consisting of at least one cell in which the activity of OASL has been reduced or eliminated.

In particular the activity of OASL has been reduced using a siRNA comprising or consisting of one of the following sequences SEQ ID NO: 45, SEQ ID NO: 45, or SEQ ID NO: 46.

There will now be described by way of example a specific mode contemplated by the Inventors. In the following description numerous specific details are set forth in order to provide a thorough understanding. It will be apparent however, to one skilled in the art, that the present invention may be practiced without limitation to these specific details. In other instances, well known methods and structures have not been described so as not to unnecessarily obscure the description.

### Example 1

### Materials and Methods

Mice, Cells and Virus

BALB/cByJ and C57BL/6J inbred mice were purchased from Charles River (L'Arbresle, France). 129/Sv/Pas and MBT/Pas mice were bred in our facilities.

Vero cells were grown in Dulbecco's modified Eagle's medium (DMEM) supplemented with 10% fetal calf serum (FCS). Primary cultures of mouse embryo fibroblast (MEF) cell lines were generated from embryos of BALB/cByJ and MBT/Pas pregnant females at day 13.5 of gestation (E13.5). Cells from single embryo were grown in separate culture dishes in DMEM supplemented with 10% FCS plus streptomycin and penicillin. Cultures were genotyped by PCR for sex determination using Smcx and Smcy genes to identify cells from male embryos [57]. Only MEFs from male embryos were used for further experiments. After 3 passages, MEFs were frozen in medium with 10% DMSO. One week before each experiment, cells were thawed to maintain MEFs at low passage numbers.

Stocks of RVFV strains ZH548 [21] and rec-ZHΔNSs [9] were produced under biosafety level 3 (BSL3) conditions. Vero cells were infected at a low MOI (10⁻³). The supernatants were harvested 72 h post-infection. Viral stocks were titrated by a standard plaque assay on Vero cells and stored at −80° C.

Mice Infection and Follow Up

Groups of fifteen matched 9- to 12-week-old males were injected intraperitoneally with 10² PFU in BSL3 cabinets. Mortality was recorded daily. For viraemia, blood samples were collected by retro-orbital puncture from 10 mice at days 1, 2 and 3 post-infection. Sera were stored at −80° C. before titration using plaque assay on Vero cells.

Cell Infection

MEFs from BALB/cByJ and MBT/Pas male embryos were plated in culture dishes 24 h prior to infection at identical densities. For virus production efficiency, cells were infected using a MOI of 1, 5 or 10 with the ZH548 strain in a low volume of media. Experiments were carried in triplicates. After one hour, cells were washed twice in PBS and grown in DMEM supplemented with 2% FCS. Supernatants were collected 15 and 20 h post-infection and stored at −80° C. For the microarray experiments, MEFs were infected using a MOI of 5. Cell monolayers were harvested 9 h later and total RNAs were extracted. For the quantitative real time reverse transcription-PCR (qRT-PCR) experiments, MEFs from three BALB/cByJ and MBT/Pas male embryos were plated at identical cell density. Twenty-four hours later, they were infected using a MOI of 5 with RVFV strain ZH548 or rec-ZHΔNSs, or with sterile media (mock-infected). Cell monolayers were harvested 3, 6, 9 and 15 h later and total RNAs were extracted.

Virus Titration

Vero cells were infected with serial dilutions of sera or cell supernatants and grown under an overlay consisting of DMEM with 2% FCS, antibiotics and 1% agarose. Four days later, cells were stained with 0.2% crystal violet in 10% formaldehyde, 20% ethanol and lytic plaques were counted.

RNA Extraction

Total RNAs from infected and mock-infected MEFs monolayers were extracted using Trizol reagent (Roche) according to manufacturer's instructions. DNA was digested by DNAse treatment using DNA-free kit (Ambion). RNA quality was assessed by electrophoresis and optic density.

Expression Microarray and Data Analysis

Gene expression profiling was performed using Affymetrix GeneChip Mouse Genome 430 2.0 Arrays (Affymetrix, Santa Clara, Calif., USA). The 430 2.0 chip contains over 27,000 unique transcripts. Samples were amplified according to the manufacturer recommended protocol. Four to 5 μg of each biotinylated cRNA preparation were fragmented and placed in a hybridization cocktail containing 4 biotinylated hybridization controls (BioB, BioC, BioD, and Cre). Samples were hybridized for 16 h. After hybridization the GeneChips were washed, stained with streptavidin-phycoerythrin, and read using an Affymetrix GeneChip fluidic station and scanner. Affymetrix raw data files were background corrected, quantile normalized and summarized using the Robust Multiarray Averaging (RMA) method [58] and transformed in log₂ values. Differentially expressed genes were filtered using dChip software [59]. Differentially expressed genes were identified as those having a fold change higher or equal to 1 on the log₂ scale between infected and non-infected MEFs from the same genetic background. This corresponds to a fold change higher or equal to 2 on the original scale. A false discovery rate <0.05 using 100 permutations was applied. Genes were further analyzed using the Functions and Disease tool from Ingenuity Pathways Analysis (Ingenuity® Systems, http://www.ingenuity.com/). This tool uses expression analysis data and assigns differently expressed genes to biological processes of interest. Results are ranked according to a p value that measures the probability that a given function is affected in the dataset. In addition, the pathway analysis reveals the most affected pathways in each dataset, among the known canonical pathways.

Quantitative RT-PCR

Equal amounts of total RNAs from infected and mock-infected MEFs 3, 6, 9 and 15 h after infection were used in a two-step qRT-PCR. To generate cDNA, RT-PCR was performed using random primers (p[dN]6, Roche) and AMV reverse transcriptase (Promega). Then, quantitative PCR was done using SYBR green master mix (Applied Biosystems) and previously described specific primers (Primer bank, http://pga.mgh.harvard.edu/primerbank/). To avoid interference due to possible polymorphism between the sequences of BALB/cByJ and MBT/Pas genomes, the hybridization site of each primer was sequenced in the BALB/cByJ and MBT/Pas genomic DNA. When polymorphism was identified, the corresponding primers were substituted for a novel pair. Table 1 shows the list of the primers used. Data were analyzed by the 2^{-ΔΔC}^{_{T}} method where changes in expression of target genes are calculated relative to internal control gene [60]. Five internal control genes, Gapdh, Rpl7, Rrm2, Tbp and Tubb5, were tested in mock- and RVFV-infected MEFs from both backgrounds at the several times post-infection. Expression of Tbp gene was similar in BALB/cByJ and MBT/Pas cells and unaffected by infection with RVFV until 9 h post-infection. Hence Tbp was selected as internal control gene for the quantitative PCR experiments.

TABLE 1
Primers used in the quantitative RT-PCR.

| | | Transcript | | |
|---|---|---|---|---|
| | | accession | | |
| Gene | Gene name | number | Primer forward (5′ to 3′) | Primer reverse (5′ to 3′) |
| Gapdh | Glyceraldehyde-3- | NM_008084 | AGGTCGGTGTGAACGGATTTG | TGTAGACCATGTAGTTGAGGTCA |
| | phosphate | | (SEQ ID NO: 11) | (SEQ ID NO: 12) |
| | dehydrogenase | | | |
| Ifit1/Isg56 | Interferon-induced | NM_008331 | GTCCGGTTAAATCCAGAAGATCC | TAGCTTTGGCAAGATGTGCTG |
| | protein with | | (SEQ ID NO: 13) | (SEQ ID NO: 14) |
| | tetratricopeptide | | | |
| | repeats 1 | | | |
| Ifit3/ifi49 | Interferon-induced | NM_010501 | CCTACATAAAGCACCTAGATGGC | ATGTGATAGTAGATCCAGGCGT |
| | protein with | | (SEQ ID NO: 15) | (SEQ ID NO: 16) |
| | tetratricopeptide | | | |
| | repeats 3 | | | |
| Ifna4 | Interferon alpha 4 | NM_010504 | TGATGAGCTACTACTGGTCAGC | GATCTCTTAGCACAAGGATGGC |
| | | | (SEQ ID NO: 17) | (SEQ ID NO: 18) |
| Ifnb1 | Interferon beta 1, | NM_010510 | CAGCTCCAAGAAAGGACGAAC | GGCAGTGTAACTCTTCTGCAT |
| | fibroblast | | (SEQ ID NO: 19) | (SEQ ID NO: 20) |
| Irf7 | Interferon regulatory | NM_016850 | GAGACTGGCTATTGGGGGAG | GACCGAAATGCTTCCAGGG |
| | factor 7 | | (SEQ ID NO: 21) | (SEQ ID NO: 22) |
| Isg15 | ISG15 ubiquitin-like | NM_015783 | CATCTATGAGGTCTTTCTGACGC | TTAGGCCATACTCCCCCAGC |
| | modifier | | (SEQ ID NO: 23) | (SEQ ID NO: 24) |
| Oasl2 | 2′-5′ oligoadenylate | NM_011854 | TTGTGCGGAGGATCAGGTACT | TGATGGTGTCGCAGTCTTTGA |
| | synthetase-like 2 | | (SEQ ID NO: 25) | (SEQ ID NO: 26) |
| Rig- | DEAD (Asp-Glu- | NM_172689 | ACTTGGGTACAACATTGCGAG | GTTCACAAGAATCTGGGGTGTC |
| I/Ddx58 | Ala-Asp) box | | (SEQ ID NO: 27) | (SEQ ID NO: 28) |
| | polypeptide 58 | | | |
| Rpl7 | Ribosomal protein | NM_011291 | GGAGCTCATCTATGAGAAGGC | AAGACGAAGGAGCTGCAGAAC |
| | L7 | | (SEQ ID NO: 29) | (SEQ ID NO: 30) |
| Rrm2 | ribonucleutide | NM_009104 | CCGAGCTGGAAAGTAAAGCG | ATGGGAAAGACAACGAAGCG |
| | reductase M2 | | (SEQ ID NO: 31) | (SEQ ID NO: 32) |
| Stat2 | Signal transducer | N_M019963 | GCATAACTTGCGAAAATTCAGCC | TCAGAATCCTTTGCTCTTCCAGA |
| | and activator of | | (SEQ ID NO: 33) | (SEQ ID NO: 34) |
| | transcription 2 | | | |
| Tbp | TATA-box binding | NM_013684 | AGAACAATCCAGACTAGCAGCA | GGGAACTTCACATCACAGCTC |
| | protein | | (SEQ ID NO: 35) | (SEQ ID NO: 36) |
| Tubb5 | Tubulin beta 5 | NM_011655 | GATCGGTGCTAAGTTCTGGGA | AGGGACATACTTGCCACCTGT |
| | | | (SEQ ID NO: 37) | (SEQ ID NO: 38) |

RNA Interference Experiments

The sequences of the stealth RNAi™ siRNA (Invitrogen, USA) used to target rf7, Isg15, Oasl2 and Rig-I are presented in Table 2.

TABEL 2
siRNA targeting Irf7, Isg15, Oasl2 and Rig-I.

| | Target | | | SEQ |
|---|---|---|---|---|
| Primer | gene | Primer sense (5′ to 3′) | Primer anti-sense (5′ to 3′) | ID NO |
| R-1 | Irf7 | GGGAUCCAGUUGAUCCGCAUAAGGU | ACCUUAUGCGGAUCAACUGGAUCCC | 39 |
| R-2 | Irf7 | CGAGUGCUGUUUGGAGACUGGCUAU | AUAGCCAGUCUCCAAACAGCACUCG | 40 |
| R-3 | Irf7 | CCAGUCCUGCUGAGCUCCCAGAUCA | UGAUCUGGGAGCUCAGCAGGACUGG | 41 |
| R-1 | Isg15 | CAGCAGCACAGUGAUGCUAGUGGUA | UACCACUAGCAUCACUGUGCUGCUG | 42 |
| R-2 | Isg15 | GCACAGUGAUGCUAGUGGUACAGAA | UUCUGUACCACUAGCAUCACUGUGC | 43 |
| R-3 | Isg15 | UGAGGUCUUUCUGACUCAGACUGUA | UACAGUCUGAGUCAGAAAGACCUCA | 44 |
| R-1 | Oasl2 | CGGGAGGUCGUCAUCAGCUUCAUUA | UAAUGAAGCUGAUGACGACCUCCCG | 45 |
| R-2 | Oasl2 | GACCAAGACAUGAUUCUGUUCUUAA | UUAAGAACAGAAUCAUGUCUUGGUC | 46 |
| R-3 | Oasl2 | CCUGCAGUGCCUGAGACGUAAAUAU | AUAUUUACGUCUCAGGCACUGCAGG | 47 |
| R-1 | Rig-I | GGGAUCCCAGCAAUGAGAAUCCUAA | UUAGGAUUCUCAUUGCUGGGAUCCC | 48 |
| R-2 | Rig-I | GCAGAACUGGAACAGGUCGUUUAUA | UAUAAACGACCUGUUCCAGUUCUGC | 49 |
| R-3 | Rig-I | GGAAGCCAUGCAACAUAUCUGUAAA | UUUACAGAUAUGUUGCAUGGCUUCC | 50 |

Three individual RNAi™ siRNA were tested for each gene target. To detect whether the siRNAs downregulated the expression of the target gene, BALB/cByJ MEFs (2×10⁵ cells) were plated onto 35 mm plates. Twenty four hours later, the cells were transfected with either stealth RNAi™ siRNA or scrambled RNAi™ siRNA. The siRNA duplexes were first incubated at room temperature for 15 min with Lipofectamine RNAiMAX (Invitrogen) in optiMEN culture media, then added to the cell plates at a final concentration of 10 nM RNAi and 1.7 μl/ml lipofectamine. Twenty four hours later, the cells were infected with RVFV strain rec-ΔNSs in 250 μl at a MOI of 5. After incubation for 1 h, the medium was removed, the plates were rinsed with PBS and 3 ml of medium with 2% FCS was added. The RNAs were extracted 6 h after infection and the levels of mRNA specific for each target gene were measured by qRT-PCR. The relative efficiency of downregulation is given as the ratio of mRNA for the target gene in cells transfected with the RNAi™ siRNA and with the scrambled RNAi™ siRNA. Experiments were done in triplicates. To test the possible induction of Ifnb1 gene by RNAi™ siRNA, Ifnb1 mRNA was measured in MEFs transfected with the RNAi™ siRNA and scrambled RNAi™ siRNA. The most efficient RNAi™ siRNA for each target gene was kept for further experiments. To test the effect of transient expression of the most efficient RNAi™ siRNA on viral production, BALB/cByJ MEFs grown in twelve 35 mm plates were transfected with either RNAi™ siRNA or scrambled RNAi™ siRNA as previously. Twenty four hours later, the cells were either infected with RVFV virulent ZH548 strain or mock-infected. At 20 h post-infection, supernatants were harvested from the culture and virus titers were determined using plaque assay on Vero cells.

Statistical Analysis

The survival curves were compared using Kaplan-Meier test [61]. For viral burden in mice, viral production in cells and qRT-PCR data, Student's t tests were performed on log₁₀-transformed data. All data were analyzed by using StatView software (SAS Institute). Data are presented as mean values±SEM.

### Example 2

### Results

Introduction

Several model rodents, rats and mice, are susceptible to RVF [12,13]. Attempts to identify genetic factors associated with host resistance to RVF in rodents have led to the discovery of different susceptibilities among eight inbred rat strains [1,4]. Indeed, Wistar-Furth (WF/mai) rats are exquisitely susceptible, while Lewis (LEW/mai) rats are highly resistant. Challenge of (WF/mai×LEW/mai) backcross rats suggested that the resistance is inherited as a major dominant locus and, accordingly, a resistant congenic line could be developed [13,15]. Genetic variability among inbred rat strains was further confirmed [16]. Altogether, experiments with the rat model demonstrated the existence of genetic determinants in RVF. To date, the identification of genetic variability in the mouse failed. In a large survey of 34 classical inbred mouse strains, all strains were found similarly susceptible [13].

The inventors have tested the susceptibility of additional inbred strains of mice. Strains recently derived from wild progenitors of different subspecies of Mus were chosen. Indeed, the available collection of wild-derived inbred strains encompasses genetic variation accumulated over ˜one million years [17], offering a larger polymorphism that classical laboratory strains, which originate from just a small number of founders and have a remarkably high level of shared ancestry largely contributed by the M. m. domesticus subspecies [18,19].

The MBT/Pas inbred strain was derived from M. m. musculus animals trapped near General Toshevo in Bulgaria in 1980; the mouse colony was later propagated by sib-mating at the Institut Pasteur [20]. We report here that MBT/Pas mice exhibit an extreme susceptibility to experimental infection with the virulent RVFV ZH548 strain compared to BALB/cByJ mice. To investigate this difference in susceptibility, we have analyzed the gene expression profile of BALB/cByJ and MBT/Pas cells following infection with RVFV. These data show that MBT/Pas cells exhibit a delayed and partial induction of type I IFN response compared with BALB/cByJ cells. Interestingly, this poorly efficient response is not caused by a difference in IFN-αs/β production, but results from inability of MBT/Pas cells to induce in due course a complete panel of interferon-stimulated genes (ISGs).

Increased Susceptibility of MBT/Pas Mice to RVFV Infection

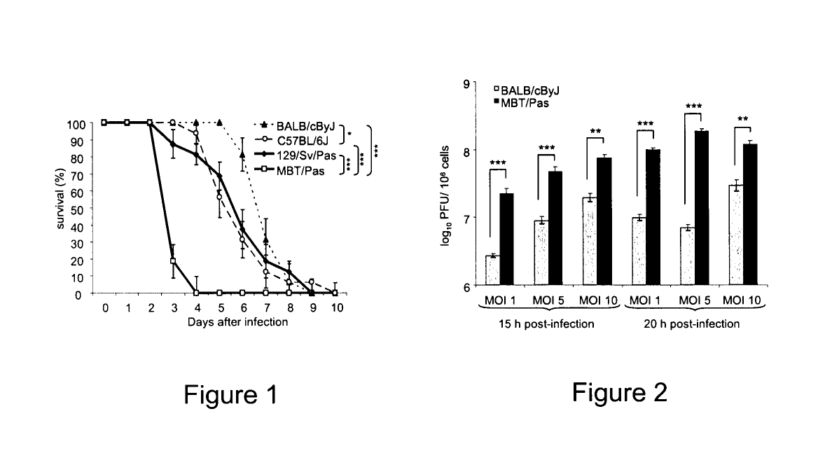

To identify polymorphisms that may influence susceptibility to RVFV amongst inbred strains of mice, we used the virulent strain ZH548, a human isolate from the Egyptian outbreak in 1977-78 [21]. Groups of male mice of various genetic backgrounds, including several classical laboratory inbred strains (BALB/cByJ, C57BL/6J and 129/Sv/Pas) and the MBT/Pas inbred strain derived from wild progenitors of the Mus m. musculus subspecies, were infected intraperitoneally with 10² plaque-forming units (PFU) (=10 LD50) of RVFV ZH548 strain. Their mortality was monitored daily for 2 weeks. In agreement with an earlier report (Peters and Anderson, 1981), classical inbred strains showed little genetic variation in susceptibility to RVF with a mean time to death of 7.19±0.21, 6.06±0.37 and 6.06±0.45 days for BALB/cByJ, C57BL/6J and 129/Sv/Pas strains, respectively. Thus C57BL/6J mice were only slightly more susceptible than BALB/cByJ mice (p=0.046). In contrast, wild-derived MBT/Pas mice were extremely susceptible to RVF: all MBT/Pas mice were dead as early as day 4 post-infection with a mean time to death of only 3.19±0.10 days (p<0.001) (FIG. 1). To test whether the earlier susceptibility of MBT/Pas mice was associated with increased viral burdens in peripheral tissues, BALB/cByJ and MBT/Pas mice (N=10) were intraperitoneally infected with 10² PFU of RVFV and bled at days 1, 2 and 3 after infection. Viral burdens in sera were measured by plaque assay in Vero cells. Infectious RVFV was detected in sera of BALB/cByJ mice starting from day 2 post-infection. In contrast, RVFV was detected earlier in sera of all MBT/Pas mice, from day 1 post-infection onwards. In addition, at day 3 post-infection the levels of RVFV in the sera of MBT/Pas mice were more than three thousand-fold higher than the levels detected in BALB/cByJ mice (10^{7.7±0.3} and 10^{4.2±0.5} PFU respectively, p=0.0015 and data not shown). Thus MBT/Pas mice produced virus earlier and their viral loads were higher compared with BALB/cByJ mice. To test whether MBT/Pas mice exhibit generalized immunodeficiency, BALB/cByJ and MBT/Pas mice were infected with 10³ PFU (=100 LD50) of West Nile virus strain IS-98-ST1. BALB/cByJ mice died whereas MBT/Pas mice survived, as reported previously [22]. These results suggest that the early susceptibility of MBT/Pas mice are caused by a selective deficiency revealed following RVFV infection.

We further investigated this defect by in vitro infection of mouse embryo fibroblasts (MEFs). Primary cell cultures from BALB/cByJ and MBT/Pas E13.5 fetuses were established. BALB/cByJ and MBT/Pas MEFs were infected at the multiplicity of infection (MOI) of 1, 5 and 10 to mimic different kinetics of the virus spread during an in vivo infection. Supernatants were analyzed for the production of infectious RVFV 15 and 20 h after infection by plaque assay. Notably, RVFV accumulation was significantly higher in MBT/Pas MEFs supernatants than in BALB/cByJ MEFs supernatants independent of the MOI and time after infection (FIG. 2). These data confirm the crucial role of the MBT/Pas genotype in controlling RVF viral spread.

BALB/cByJ MEFs Response Against RVFV Infection Includes Activation of the Type I Interferon Pathway

To examine the global effect of RVFV infection and cell's ability to respond to it, we analyzed the gene expression profile of RVFV-infected BALB/cByJ and MBT/Pas MEFs using microarray experiments. A MOI of 5 was used to insure that every cell would be infected by an infectious particle. Total RNAs from three culture dishes of either mock- or RVFV-infected MEFs from BALB/cByJ and MBT/Pas embryos were extracted at 9 h after infection, a time point at which the antiviral response has been shown to be detectable and the viral-induced inhibition of transcription is still low [8]. Total RNAs were hybridized to Affymetrix MOE 430 2.0 chips.

Data were normalized and transformed in log₂ values. Fold changes between infected and mock-infected MEFs were calculated. A gene was considered to be differentially regulated by RVFV if its expression in infected cells was at least twofold higher (for upregulated genes) or twofold lower (for downregulated genes) than its expression in mock-infected cells of the same genetic background. A false discovery rate of 5% based on 100 permutations was applied. Principal component analysis of all regulated genes confirmed that the expression changes were true biological variations and were not caused by variations in experimental conditions (data not shown). The complete microarray data have been deposited in NCBI's Gene Expression Omnibus [23] and are accessible through GEO Series accession number GSE18064 (http://www.ncbi.nlm.nih.gov/geo/query/acc.cgi?acc=GSE18064).

Quantitative analysis of the data showed that only 229 unique genes (0.82% of cellular transcripts) fulfilled these criteria in BALB/cByJ MEFs (Table 3).

TABLE 3
Genes whose expression is modulated in BALB/cBy MEFs at 9 h post-infection with RVFV.

| | | Accession | Fold change in infected |
|---|---|---|---|
| Gene | gene name | number* | BALB/cByJ cells** |
| Ifit1 | Interferon-induced protein with tetratricopeptide | NM_008331 | 4.87 |
| | repeats 1 | | |
| Usp18 | Usp18: ubiquitin specific peptidase 18 | NM_011909 | 4.80 |
| Oasl2 | 2′-5′ oligoadenylate synthetase-like 2 | BQ033138 | 4.63 |
| Gbp3 | Guanylate nucleotide binding protein 3 | NM_018734 | 3.99 |
| Ligp1 | Interferon inducible gtpase 1 | BM239828 | 3.91 |
| BC013672 | Cdna sequence BC013672 | BC013672 | 3.87 |
| Ifi44 | Interferon-induced protein 44 | BB329808 | 3.75 |
| Isg15 | ISG15 ubiquitin-like modifier | AK019325 | 3.41 |
| Fbxo39 | F-box protein 39 | BB645745 | 2.90 |
| Ifih | Interferon induced with helicase C domain 1 | AY075132 | 2.78 |
| Gvin1 | Gtpase, very large interferon inducible 1 | BM243571 | 2.72 |
| Ifi203 | Interferon activated gene 203 | NM_008328 | 2.63 |
| Stat2 | Stat2: signal transducer and activator of transcription 2 | AF088862 | 2.56 |
| D14Ertd668e | DNA segment, Chr 14, ERATO Doi 668, expressed | AV280841 | 2.53 |
| Rtp4 | Receptor transporter protein 4 | BC024872 | 2.52 |
| Gbp1 | Guanylate nucleotide binding protein 1 | NM_010259 | 2.52 |
| Ligp2 | Interferon inducible gtpase 2 | NM_019440 | 2.51 |
| Stat1 | Signal transducer and activator of transcription 1 | AW214029 | 2.50 |
| 2010003H20Rik | RIKEN cdna 2010003H20 gene | AK008065 | 2.43 |
| Saa3 | Serum amyloid A 3 | NM_011315 | 2.42 |
| Igtp | Interferon gamma induced gtpase | NM_018738 | 2.42 |
| Gbp2 | Guanylate nucleotide binding protein 2 | NM_010260 | 2.36 |
| Herc5 | Hect domain and RLD 5 | AW208668 | 2.33 |
| Dhx58 | DEXH (Asp-Glu-X-His) box polypeptide 58 | AF316999 | 2.30 |
| 6820431F20Rik | RIKEN cdna 6820431F20 gene | AI643819 | 2.22 |
| 9530057J20Rik | EG665787RIKEN cdna 9530057J20 gene | BE945468 | 2.16 |
| Cxcl10 | Chemokine (C-X-C motif) ligand 10 | NM_021274 | 2.13 |
| Gbp6 | Guanylate binding protein 6 | BC010229 | 2.11 |
| Isg20 | Interferon-stimulated protein | BC022751 | 2.11 |
| Ifi205 /// Mnda | Interferon activated gene 205 /// myeloid cell nuclear | AI481797 | 2.11 |
| | differentiation antigen | | |
| Ifi202b | Interferon activated gene 202B | AV229143 | 2.02 |
| Ifit3 | Ifit3interferon-induced protein with tetratricopeptide | NM_010501 | 1.99 |
| | repeats 3 | | |
| Ptprj | Protein tyrosine phosphatase, receptor type, J | D83204 | 1.97 |
| Irf7 | Irf7interferon regulatory factor 7 | NM_016850 | 1.97 |
| BC006779 | Cdna sequence BC006779 | BE853170 | 1.97 |
| Brd4 /// Bst2 | Bromodomain containing 4 /// bone marrow stromal cell | BC008532 | 1.95 |
| | antigen 2 | | |
| Parp9 | Poly (ADP-ribose) polymerase family, member 9 | NM_030253 | 1.90 |
| Rnf213 | Ring finger protein 213 | AW556558 | 1.88 |
| Plec1 | Plectin 1 | BI525140 | 1.85 |
| | Transcribed locus | AW491006 | 1.84 |
| Ifi204 | Interferon activated gene 204 | NM_008329 | 1.81 |
| Icam1 | Intercellular adhesion molecule | BC008626 | 1.81 |
| Oas1a | 2′-5′ oligoadenylate synthetase 1A | BC018470 | 1.79 |
| Mpa2l | Macrophage activation 2 like | BG092512 | 1.78 |
| Samd9l | Sterile alpha motif domain containing 9-like | BB145092 | 1.76 |
| Serpina3n | Serine (or cysteine) peptidase inhibitor, clade A, | NM_009252 | 1.75 |
| | member 3N | | |
| Parp12 | Poly (ADP-ribose) polymerase family, member 12 | BM227980 | 1.75 |
| Il6 | Interleukin 6 | NM_031168 | 1.75 |
| Nmi | N-myc (and STAT) interactor | BC002019 | 1.74 |
| Ifi35 | Interferon-induced protein 35 | AW986054 | 1.74 |
| Ifi27 | Interferon, alpha-inducible protein 27 | AY090098 | 1.74 |
| Psmb8 | Proteasome (prosome, macropain) subunit, beta type 8 | NM_010724 | 1.73 |
| | (large multifunctional peptidase 7) | | |
| | Transcribed locus | AW124751 | 1.73 |
| EG667823 | Predicted gene EG667823 | BI653857 | 1.72 |
| Parp14 | Poly (ADP-ribose) polymerase family, member 14 | BC021340 | 1.70 |
| Ddx58 | DEAD (Asp-Glu-Ala-Asp) box polypeptide 58 | BG063981 | 1.69 |
| | Transcribed locus | BQ086474 | 1.68 |
| Prkar2b | Protein kinase, camp dependent regulatory, type II | BB216074 | 1.67 |
| | beta | | |
| Plac8 | Placenta-specific 8 | AF263458 | 1.65 |
| Oasl1 | 2′-5′ oligoadenylate synthetase-like 1 | AB067533 | 1.65 |
| 2900019G14Rik | RIKEN cdna 2900019G14 gene | AW122190 | 1.65 |
| Micalcl | MICAL C-terminal like | AK014911 | 1.64 |
| Lgals3bp | Lectin, galactoside-binding, soluble, 3 binding protein | NM_011150 | 1.64 |
| Irgm | Immunity-related gtpase family, M | NM_008326 | 1.64 |
| ENSMUSG00000073237 | Predicted gene ENSMUSG00000073237 | BM240795 | 1.59 |
| Tor3a | Torsin family 3, member A | AK009693 | 1.59 |
| Cxcl2 | Chemokine (C-X-C motif) ligand 2 | NM_009140 | 1.57 |
| Eif2ak2 | Eukaryotic translation initiation factor 2-alpha kinase 2 | BE911144 | 1.57 |
| Ampd1 | Adenosine monophosphate deaminase 1 (isoform M) | AW146181 | 1.57 |
| Tgtp | T-cell specific gtpase | NM_011579 | 1.56 |
| Dtx3l | Deltex 3-like (Drosophila) | AV327407 | 1.56 |
| Map3k8 | Mitogen activated protein kinase kinase kinase 8 | NM_007746 | 1.55 |
| 2310016F22Rik | RIKEN cdna 2310016F22 gene | BC020489 | 1.55 |
| Isgf3g | Interferon dependent positive acting transcription factor | NM_008394 | 1.51 |
| | 3 gamma | | |
| Casp4 | Caspase 4, apoptosis-related cysteine peptidase | NM_007609 | 1.50 |
| | Transcribed locus | BI647951 | 1.50 |
| Prelp | Proline arginine-rich end leucine-rich repeat | NM_054077 | 1.47 |
| C1s | Complement component 1, s subcomponent | BC022123 | 1.46 |
| | Transcribed locus | AW111920 | 1.46 |
| Amy1 | Amylase 1, salivary | NM_007446 | 1.44 |
| 2010305C02Rik | RIKEN cdna 2010305C02 gene | AK008522 | 1.44 |
| Serpina3g | Serine (or cysteine) peptidase inhibitor, clade A, | BC002065 | 1.42 |
| | member 3G | | |
| Gramd1c | GRAM domain containing 1C | AV255657 | 1.40 |
| Arid5b | AT rich interactive domain 5B (Mrf1 like) | BB699910 | 1.39 |
| | Mm, 26204, 1 | BG069797 | 1.39 |
| Trim21 | Tripartite motif protein 21 | BC010580 | 1.38 |
| Tgfb2 | Transforming growth factor, beta 2 | BF144658 | 1.38 |
| Rarres1 | Retinoic acid receptor responder (tazarotene induced) | BB035017 | 1.38 |
| | 1 | | |
| H2-T23 | Histocompatibility 2, T region locus 23 | NM_010398 | 1.37 |
| Prpf38a | PRP38 pre-mrna processing factor 38 (yeast) domain | AV320497 | 1.36 |
| | containing A | | |
| 9530028C05 | Hypothetical protein 9530028C05 | BQ175154 | 1.36 |
| Plscr2 | Phospholipid scramblase 2 | NM_008880 | 1.34 |
| H2-T10 /// H2-T22 /// | Histocompatibility 2, T region locus 10 /// | NM_010395 | 1.31 |
| H2-T9 | histocompatibility 2, T region locus 22 /// | | |
| | histocompatibility 2, T region locus 9 | | |
| Islr | Immunoglobulin superfamily containing leucine-rich | NM_012043 | 1.29 |
| | repeat | | |
| C3 | Complement component 3 | K02782 | 1.27 |
| Sfrp1 | Secreted frizzled-related protein 1 | BI658627 | 1.26 |
| BC032204 | Cdna sequence BC032204 | BB113173 | 1.25 |
| 1700056N10Rik | RIKEN cdna 1700056N10 gene | AK006816 | 1.25 |
| Tgm2 | Transglutaminase 2, C polypeptide | BC016492 | 1.24 |
| Gcg | Glucagon | AF276754 | 1.24 |
| Tmem30c | Transmembrane protein 30C | AK016747 | 1.23 |
| Trim30 | Tripartite motif protein 30 | AF220015 | 1.22 |
| Agrn | Agrin | BM208224 | 1.22 |
| Aoc3 | Amine oxidase, copper containing 3 | NM_009675 | 1.21 |
| Ccl11 | Small chemokine (C-C motif) ligand 11 | NM_011330 | 1.20 |
| A430036N23 product | 0 day neonate thymus cdna, RIKEN full-length | BB200911 | 1.20 |
| | enriched library, clone: A430036N23 product: junction | | |
| | cell adhesion molecule 3, full insert sequence | | |
| Rsad2 | Radical S-adenosyl methionine domain containing 2 | BB132493 | 1.20 |
| H2-K1 | Histocompatibility 2, K1, K region | BC011306 | 1.19 |
| | Mm, 38315, 1 | AA415783 | 1.19 |
| Rspo1 | R-spondin homolog (Xenopus laevis) | NM_138683 | 1.18 |
| Igfbp3 | Insulin-like growth factor binding protein 3 | AV175389 | 1.18 |
| LOC100044874 | Similar to H-2K(d) antigen | S70184 | 1.17 |
| | Mm, 120444, 1 | BB154367 | 1.17 |
| | Transcribed locus | BM217870 | 1.17 |
| Tap1 | Transporter 1, ATP-binding cassette, sub-family B | AW048052 | 1.16 |
| | (MDR/TAP) | | |
| Rc3h2 | Ring finger and CCCH-type zinc finger domains 2 | AA709668 | 1.15 |
| LOC100045864 | Similar to HLA-G protein | M83244 | 1.15 |
| Mlkl | Mixed lineage kinase domain-like | AK018636 | 1.14 |
| H2-D1 /// H2-K1 | Histocompatibility 2, D region locus 1 /// | L23495 | 1.13 |
| | histocompatibility 2, K1, K region | | |
| A730037C10Rik | RIKEN cdna A730037C10 gene | BB249985 | 1.13 |
| Adamts5 | Disintegrin-like and metallopeptidase (reprolysin type) | BB658835 | 1.12 |
| | with thrombospondin type 1 motif, 5 (aggrecanase-2) | | |
| Tnfaip3 | Tumor necrosis factor, alpha-induced protein 3 | BM241351 | 1.12 |
| Scara3 | Scavenger receptor class A, member 3 | BC026446 | 1.12 |
| | Mm, 132282, 1 | BB198687 | 1.12 |
| Zbp1 | Zbp1Z-DNA binding protein 1 | AK008179 | 1.11 |
| Lgals9 | Lectin, galactose binding, soluble 9 | NM_010708 | 1.11 |
| A930030B08Rik | RIKEN cdna A930030B08 gene | BB281168 | 1.11 |
| Tdrd7 | Tudor domain containing 7 | BC025099 | 1.10 |
| Hspb6 | Heat shock protein, alpha-crystallin-related, B6 | BB755506 | 1.10 |
| Enc1 | Ectodermal-neural cortex 1 | BM120053 | 1.10 |
| Diap3 | Diaphanous homolog 3 (Drosophila) | NM_019670 | 1.10 |
| Cenpe | Centromere protein E | BG068387 | 1.09 |
| Adar | Adenosine deaminase, RNA-specific | AF291876 | 1.09 |
| Pcf11 | Cleavage and polyadenylation factor subunit homolog | AV374246 | 1.09 |
| | (S. cerevisiae) | | |
| Slfn9 | Schlafen 9 | BI647893 | 1.08 |
| Trim25 | Tripartite motif protein 25 | D63902 | 1.07 |
| Pcaf | P300/CBP-associated factor | AV094898 | 1.07 |
| Cxcl11 | Chemokine (C-X-C motif) ligand 11 | NM_019494 | 1.07 |
| Tmem106a | Transmembrane protein 106A | BC022145 | 1.06 |
| Lbp | Lipopolysaccharide binding protein | NM_008489 | 1.06 |
| 7420416P19 product | In vitro fertilized eggs cdna, RIKEN full-length enriched | BB700210 | 1.05 |
| | library, clone: 7420416P19 product: unclassifiable, full | | |
| | insert sequence | | |
| Gipc2 | GIPC PDZ domain containing family, member. 2 | NM_016867 | 1.04 |
| A630077B13Rik | RIKEN cdna A630077B13 gene | BB239429 | 1.04 |
| Aebp2 | AE binding protein 2 | NM_009637 | 1.03 |
| Slco3a1 | Solute carrier organic anion transporter family, member | BM237089 | 1.02 |
| | 3a1 | | |
| Hpx | Hemopexin | BC011246 | 1.02 |
| 9830115L13Rik | RIKEN cdna 9830115L13 gene | BB757349 | 1.02 |
| | Transcribed locus | BB045448 | 1.01 |
| Vcam1 | Vascular cell adhesion molecule 1 | BB250384 | 1.00 |
| Fpr-rs2 | Formyl peptide receptor, related sequence 2 | NM_008039 | 1.00 |
| 9130213B05Rik | RIKEN cdna 9130213B05 gene | BC006604 | 1.00 |
| | Mm, 172186, 1 | BG071402 | 1.00 |
| Obfc2a | Oligonucleotide/oligosaccharide-binding fold containing | AV313559 | −1.00 |
| | 2A | | |
| Mcl1 | Myeloid cell leukemia sequence 1 | BC003839 | −1.01 |
| Tmem186 | Transmembrane protein 186 | AW488762 | −1.02 |
| Snx5 | Sorting nexin 5 | BG067008 | −1.02 |
| Btg1 | B-cell translocation gene 1, anti-proliferative/ | L16846 | −1.02 |
| 2700023E23Rik | RIKEN cdna 2700023E23 gene | BB822655 | −1.02 |
| 2210411K19Rik | RIKEN cdna 2210411K19 gene | BI694945 | −1.02 |
| Zxdc | ZXD family zinc finger C | BB238025 | −1.04 |
| 4833424O12Rik | RIKEN cdna 4833424O12 gene | AK014762 | −1.04 |
| Irf2bp2 | Interferon regulatory factor 2 binding protein 2 | BB183385 | −1.05 |
| Homer1 | Homer homolog 1 (Drosophila) | BB398124 | −1.05 |
| 9130004J05Rik | RIKEN cdna 9130004J05 gene | BB748887 | −1.05 |
| | Mm, 178453, 1 | BM201095 | −1.05 |
| | Transcribed locus | BI134319 | −1.05 |
| Dusp5 | Dual specificity phosphatase 5 | BB442784 | −1.06 |
| Cxcr6 | Chemokine (C-X-C motif) receptor 6 | AF301018 | −1.06 |
| Rtn4 | Reticulon 4 | BB648600 | −1.07 |
| Pus3 | Pseudouridine synthase 3 | NM_023292 | −1.07 |
| Esd | Esterase D/formylglutathione hydrolase | BM248597 | −1.07 |
| Cited2 | Cbp/p300-interacting transactivator, with Glu/Asp-rich | Y15163 | −1.07 |
| | carboxy-terminal domain, 2 | | |
| Prkg2 | Protein kinase, cgmp-dependent, type II | BB823350 | −1.08 |
| 6720467C03Rik | RIKEN cdna 6720467C03 gene | AV350862 | −1.08 |
| 2610203C20Rik | RIKEN cdna 2610203C20 gene | BM220421 | −1.08 |
| Klf2 | Kruppel-like factor 2 (lung) | NM_008452 | −1.10 |
| Egr3 | Early growth response 3 | AV346607 | −1.10 |
| Klf2 | Kruppel-like factor 2 (lung) | NM_008452 | −1.10 |
| Trib2 | Tribbles homolog 2 (Drosophila) | BB354684 | −1.11 |
| Snora65 | Small nucleolar RNA, H/ACA box 65 | BG807990 | −1.11 |
| Cpsf6 | Cleavage and polyadenylation specific factor 6 | BB335087 | −1.11 |
| Gspt2 | G1 to S phase transition 2 | NM_008179 | −1.12 |
| LOC100040608 /// | Similar to Fanconi anemia, complementation group F | BB667193 | −1.13 |
| LOC100046423 | | | |
| A530054K11Rik | RIKEN cdna A530054K11 gene | BG072966 | −1.13 |
| | Mm, 213227, 1 | BB051952 | −1.13 |
| | Mm, 218032, 1 | BE334274 | −1.13 |
| Klf6 | Kruppel-like factor 6 | NM_011803 | −1.14 |
| 4933439C20Rik | RIKEN cdna 4933439C20 gene | BB504983 | −1.14 |
| Rpl3 | Ribosomal protein L3 | BG073445 | −1.15 |
| Josd3 | Josephin domain containing 3 | AV167760 | −1.15 |
| Prdm2 | PR domain containing 2, with 2NF domain | BM226301 | −1.16 |
| Ncam1 | Neural cell adhesion molecule 1 | BM201198 | −1.16 |
| Clcf1 | Cardiotrophin-like cytokine factor 1 | BB825816 | −1.16 |
| Snhg3 | Small nucleolar RNA host gene (non-protein coding) 3 | BI082172 | −1.17 |
| | Mm, 217822, 1 | BG060698 | −1.20 |
| Npal1 | NIPA-like domain containing 1 | AK014427 | −1.22 |
| Jun | Jun oncogene | NM_010591 | −1.22 |
| 2610019E17Rik | RIKEN cdna 2610019E17 gene | BF660912 | −1.22 |
| Errfi1 | ERBB receptor feedback inhibitor 1 | NM_133753 | −1.25 |
| Sertad4 | SERTA domain containing 4 | BQ174721 | −1.25 |
| Flrt2 | Fibronectin leucine rich transmembrane protein 2 | AW555664 | −1.25 |
| Ints12 | Integrator complex subunit 12 | AK015941 | −1.27 |
| | Mm, 212452, 1 | BB710847 | −1.28 |
| Has2 | Hyaluronan synthase 2 | NM_008216 | −1.34 |
| Snord22 | Small nucleolar RNA, C/D box 22 | BF163381 | −1.35 |
| Fbln2 | Fibulin 2 | AV022444 | −1.35 |
| Mllt11 | Myeloid/lymphoid or mixed-lineage leukemia (trithorax | NM_019914 | −1.36 |
| | homolog, Drosophila); translocated to, 11 | | |
| 6720475J19Rik | RIKEN cdna 6720475J19 gene | AW549928 | −1.39 |
| Tnc | Tenascin C | BB003393 | −1.41 |
| Cyr61 | Cysteine rich protein 61 | BB533736 | −1.41 |
| Mat2a | Methionine adenosyltransferase II, alpha | BG065061 | −1.42 |
| | Mm, 182729, 1 | AW553625 | −1.44 |
| Rhob | Ras homolog gene family, member B | BC018275 | −1.45 |
| Rps9 | Ribosomal protein S9 | AK013903 | −1.46 |
| Prmt6 | Protein arginine N-methyltransferase 6 | BC022899 | −1.47 |
| Hmox1 | Heme oxygenase (decycling) 1 | NM_010442 | −1.47 |
| Zic1 | Zinc finger protein of the cerebellum 1 | BB361162 | −1.48 |
| Rai14 | Retinoic acid induced 14 | BB308974 | −1.49 |
| Hspa8 | Heat shock protein 8 | AK004608 | −1.49 |
| Gas5 | Growth arrest specific 5 | AW547050 | −1.50 |
| 1110061A14Rik | RIKEN cdna 1110061A14 gene | BI654939 | −1.51 |
| Cblb | Casitas B-lineage lymphoma b | BB205662 | −1.53 |
| Ptgs2 | Prostaglandin-endoperoxide synthase 2 | M94967 | −1.54 |
| Sema4f | Sema domain, immunoglobulin domain (Ig), TM | BB271145 | −1.56 |
| | domain, and short cytoplasmic domain | | |
| Bnc1 | Basonuclin 1 | U88064 | −1.56 |
| Foxc2 | Forkhead box C2 | NM_013519 | −1.58 |
| Matr3 | Matrin 3 | BI249188 | −1.67 |
| 2310043N10Rik | RIKEN cdna 2316043N10 gene | AK018202 | −1.71 |
| Slc20a1 | Solute carrier family 20, member 1 | BB465699 | −1.94 |
| Dusp4 | Dual specificity phosphatase 4 | AK012530 | −2.03 |

*Accession number given by Dchip software
**The fold changes in mRNA levels in RVF virus-infected cells relative to mock-infected cells are displayed in log2 values

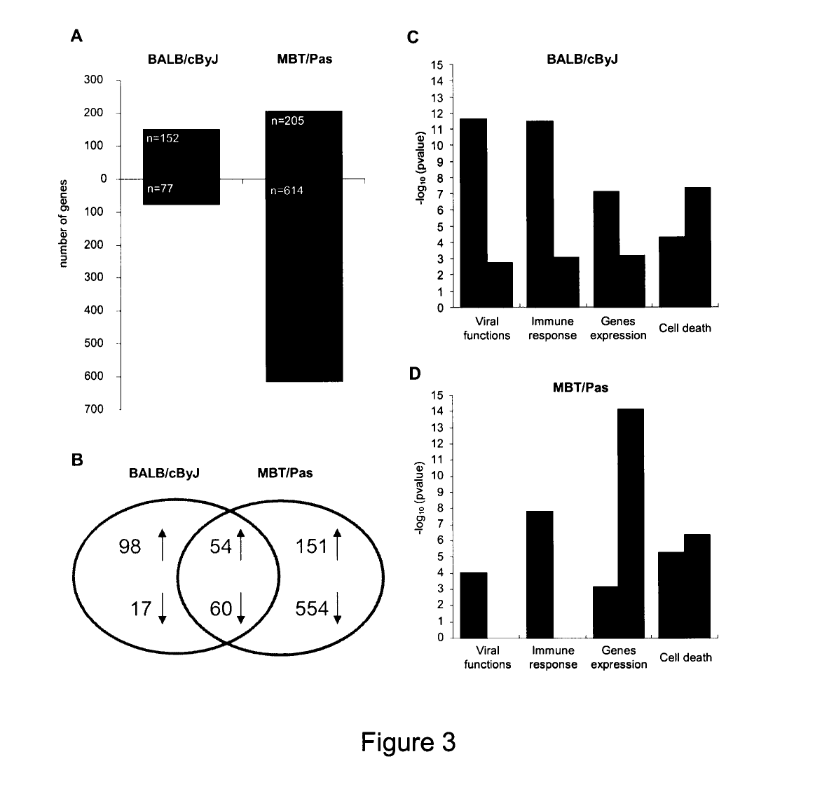

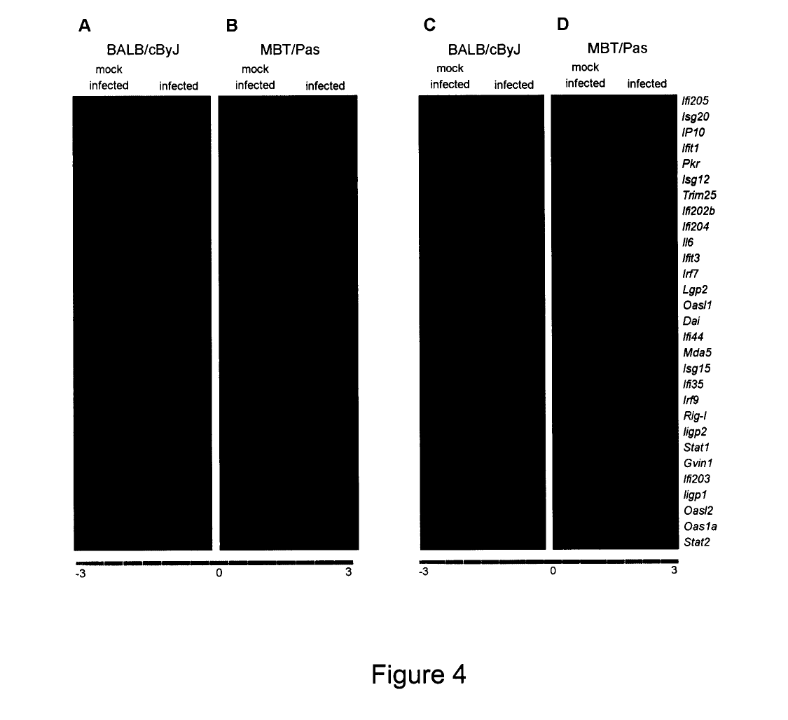

Of these genes, 152 were upregulated with a maximal fold change increase of 29.2 whereas 77 were downregulated with a maximal fold change decrease of −4.1 (Table S1; FIG. 3A and B). FIG. 4A shows a heat map representation of these 229 genes whose expression was modulated by RVFV infection in BALB/cByJ MEFs. To identify the main functions differentially regulated by RVFV, we used the Ingenuity Pathway Analysis database. In BALB/cByJ MEFs, the upregulated genes were firstly related to ‘viral functions’ and ‘immune response’ categories while the downregulated genes were mostly related to the ‘cell death’ category (FIG. 3 C).

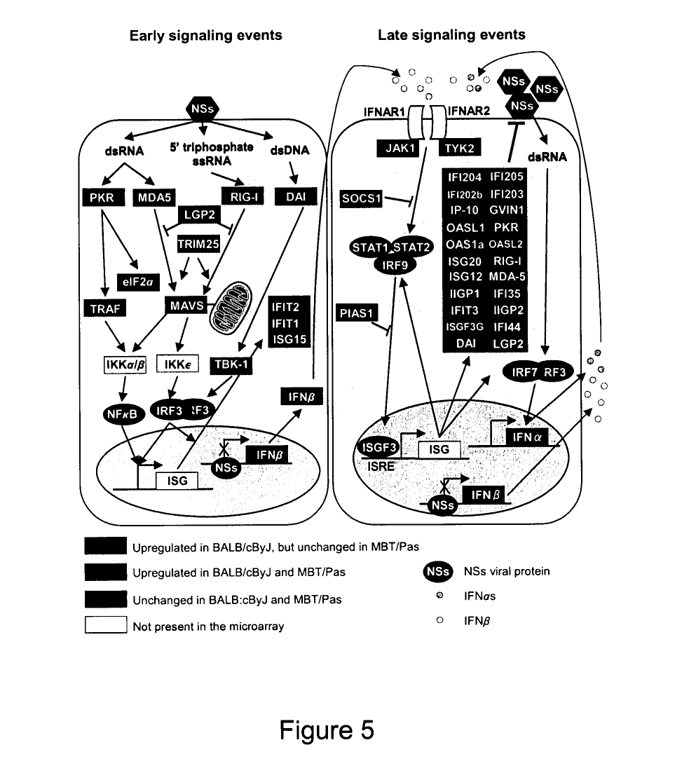

The pathways leading to induction of ISGs were strongly stimulated by RVFV and many genes encoding components of this mechanism were upregulated in BALB/cByJ MEFs (FIG. 4C). The induction of ISGs expression is complex and involves two successive phases [24,25]. During an early phase, ISGs are produced by cells in direct response to virus infection, following the triggering of the pattern recognition receptors (PRRs) that eventually leads to IFN regulatory factor 3 (IRF3) phosphorylation, and IFN-β production. A limited number of ISGs, which are IRF3 direct targets, are also produced in this phase. During the late phase, this initially produced IFN-β stimulates the type I IFN receptor to activate ISGF3, the trimeric complex formed by IFN regulatory factor 9 (IRF9), signal transducers and activators of transcription 1 and 2 (STAT1 and STAT2), that binds specific sites in the promoters of ISGs and triggers transcription. As shown in FIG. 5, the various genes that were upregulated in BALB/cByJ cells in response to RVFV infection include genes for cytoplasmic PRRs, including the dsRNA-dependent protein kinase PKR/EIF2AK2, the RNA helicases RIG-I and MDA5, and the cytosolic DNA sensor DAI/ZBP1. Irf3 mRNA is expressed constitutively in MEFs [26]. Irf3 gene was expressed at high levels before RVFV infection and its expression was not changed by the infection (data not shown). The gene for interferon-induced protein with tetratricopeptide repeats 1 IFIT1/ISG56 is a direct target of IRF3 [27]; Ifit1 gene was upregulated following RVFV infection. Components of the late phase were also upregulated, including the genes for the three ISGF3 subunits, IRF9, STAT1 and STAT2. IFN regulatory factor 7 (IRF7) transcription factor can activate both Ifna and Ifnb1 genes in response to viruses [24]; Irf7 gene was upregulated by RVFV. The infection stimulated a number of genes encoding known antiviral effectors, including the interferon stimulated exonuclease ISG20 [28], several p65 GTP-binding proteins (GBP1 to GBP3, GBP6) [29,30] and p47 GTPases proteins (IGTP, IRGM1, IIGP1, IIGP2, TGTP) [30], and 2′-5′-oligoadenylate synthetases (OASs), namely OAS1A, OASL1 and OASL2 [31]. Other interferon-induced genes, such as Isg12/Ifi27 [32] and several members of the P200 protein family (Ifi202b, Ifi203, Ifi204 and Ifi205) [33], were activated following infection. In addition to the IFN-αs/β pathways, RVFV also stimulated the expression of genes encoding inflammatory molecules such as IL6 cytokine and CXCL2, CXCL10 and CXCL11 chemokines. Table 4 gives a summary of several upregulated genes in infected BALB/cByJ MEFs that are implicated in the innate immune response.

TABLE 4
Upregulation of the innate immune response genes in RVFV-infected
BALB/cByJ and MBT/Pas MEFs as shown by microarray experiments.

| | | | Fold change in | Fold change in |
|---|---|---|---|---|
| | | Transcript accession | infected BALB/cByJ | infected MBT/Pas |
| Gene | Gene name | number | cells* | cells* |
| Dai/Zbp1 | Z-DNA binding protein | NM_021394 | 1.11 | 0.01 |
| | 1 | | | |
| Gvin1 | GTPase, very large | NM_029000 | 2.72 | −0.09 |
| | interferon inducible 1 | | | |
| Ifi202b | Interferon activated | NM_011940 | 1.90 | 0.24 |
| | gene 202B | | | |
| Ifi203 | Interferon activated | NM_008328 | 2.63 | 0.64 |
| | gene 203 | | | |
| Ifi204 | Interferon activated | NM_008329 | 1.81 | 0.21 |
| | gene 204 | | | |
| Ifi205 | Interferon activated | NM_172648 | 2.11 | 0.94 |
| | gene 205 | | | |
| Ifi35 | Interferon-induced | NM_027320 | 1.36 | 0.59 |
| | protein 35 | | | |
| Ifi44 | Interferon-induced | NM_133871 | 3.75 | 1.48 |
| | protein 44 | | | |
| Ifit1/Isg56 | Interferon-induced | NM_008331 | 4.87 | 3.95 |
| | protein with | | | |
| | tetratricopeptide | | | |
| | repeats 1 | | | |
| Ifit3/Ifi49 | Interferon-induced | NM_010501 | 1.99 | 0.88 |
| | protein with | | | |
| | tetratricopeptide | | | |
| | repeats 3 | | | |
| Iig1 | Interferon inducible | AF194871 | 2.41 | 0.39 |
| | GTPase 1 | | | |
| Iigp2/Irgm2 | Interferon inducible | NM_019440 | 2.51 | 0.23 |
| | GTPase 2 | | | |
| Il6 | Interleukin 6 | NM_031168 | 1.75 | 1.53 |
| Ip10/Cxcl10 | Chemokine (C-X-C | NM_021274 | 2.13 | 2 09 |
| | motif) ligand 10 | | | |
| Irf7 | Interferon regulatory | NM_016850 | 1.97 | 0.39 |
| | factor 7 | | | |
| Isg12/Ifi27l2a | Interferon, alpha- | NM_029803 | 1.74 | −0.19 |
| | inducible protein 27 | | | |
| Isg15 & | ISG15 ubiquitin-like | NM_015783 | 3.41 | 1.54 |
| Gm9708** | modifier | | | |
| Isg20 | Interferon-stimulated | NM_020583 | 2.11 | 3.49 |
| | protein | | | |
| Irf9/Isgf3g | Interferon dependent | NM_008394 | 1.51 | 0.73 |
| | positive acting | | | |
| | transcription factor 3 | | | |
| | gamma | | | |
| Lpg2/Dhx58 | DEXH (Asp-Glu-X- | NM_030150 | 2.30 | 1.02 |
| | His) box polypeptide | | | |
| | 58 | | | |
| Mda5/Ifih1 | Interferon induced with | NM_027835 | 2.78 | 1.71 |
| | helicase C domain 1 | | | |
| Oas1a | 2′-5′ oligoadenylate | NM_145211 | 1.79 | 0.26 |
| | synthetase 1A | | | |
| Oasl1 | 2′-5′ oligoadenylate | NM_145209 | 1.65 | 0.81 |
| | synthetase-like 1 | | | |
| Oasl2 | 2′-5′ oligoadenylate | NM_011854 | 4.63 | 0.14 |
| | synthetase-like 2 | | | |
| Pkr/Eif2ak2 | Eukaryotic translation | NM_011163 | 1.92 | 0.51 |
| | initiation factor 2-alpha | | | |
| | kinase 2 | | | |
| Rig-1/Ddx58 | DEAD (Asp-Glu-Ala- | NM_172689 | 1.38 | 0.35 |
| | Asp) box polypeptide | | | |
| | 58 | | | |
| Stat1 | Signal transducer and | NM_009283 | 1.81 | 0.36 |
| | activator of | | | |
| | transcription 1 | | | |
| Stat2 | Signal transducer and | NM_019963 | 2.56 | 0.33 |
| | activator of | | | |
| | transcription 2 | | | |
| Trim25 | Tripartite motif protein | NM_009546 | 1.06 | 0.71 |
| | 25 | | | |

*The fold changes in mRNA levels in RVFV-infected cells relative to mock-infected cells are displayed in log₁ values.
**The microarray probe for Isg15 also hybridizes with the hypothetical Gm9708 (see results).

MBT/Pas MEFs Exhibit Weak IFN-Dependent Response Against RVFV Infection

MBT/Pas cells produced higher viral titers than BALB/cByJ MEFs. To investigate the mechanism that renders MBT/Pas cells more permissive to the virus, gene expression in mock- and RVFV-infected MBT/Pas MEFs was analysed.

Quantitative analysis showed that 819 genes were differently regulated in MBT/Pas MEFs by RVFV. Of these, 205 were upregulated while 614 were downregulated (FIGS. 3A and 3B). MBT/Pas MEFs produced higher numbers of infectious particles and presumably accumulated larger amounts of the viral NSs protein. Since NSs inhibits Ifnb1 transcription [9,34], it was conceivable that downregulation of this high number of genes is a direct effect of NSs. In contrast with this hypothesis, none of the downregulated genes are related to either ‘viral functions’ or ‘immune response’ categories, while the upregulated genes show an over-representation of the ‘immune response’ and ‘viral functions’, suggesting that MBT/Pas cells were still able to elicit an innate immune response (FIG. 3D). However, these categories are less significantly represented in MBT/Pas than in BALB/cByJ response (compare FIG. 3C with FIG. 3D). Differences between strains became apparent when the lists of genes differentially regulated in BALB/cByJ and MBT/Pas MEFs were compared. Seventy eight percent of the genes that were downregulated in BALB/cByJ MEFs were also downregulated in MBT/Pas MEFs. In contrast, only 35% of the genes upregulated in BALB/cByJ MEFs were also upregulated in MBT/Pas MEFs (FIG. 3B). Altogether, these data suggest that MBT/Pas MEFs are not able to elicit the same antiviral response when compared with BALB/cByJ MEFs.

The inventors then went on to investigate amongst the genes that were upregulated in BALB/cByJ MEFs, those that are typically induced after type I IFN stimulation (FIG. 4C). Table 4 provides a list of 29 of these genes and their fold changes in BALB/cByJ and MBT/Pas MEFs. Many of these genes were not upregulated by RVFV in MBT/Pas cells (also compare FIGS. 4C and 4D). Indeed, only 8 genes, namely Mda5, Isg20, ISG15 ubiquitin-like modifier gene (Isg15), Ifit1, RNA helicase LGP2 (Lpg2/Dhx58), Il6, Interferon-induced protein 44 (Ifi44), and Cxcl10, were upregulated in MBT/Pas MEFs. Noteworthy, genes for the RNA helicase RIG-I, dsRNA-dependent protein kinase PKR, IRF regulatory factor IRF7, the three ISGF3 subunits, and three oligoadenylate synthetase (OAS1a, OASL1 and OASL2) were stimulated at significantly lower levels in MBT/Pas MEFs than in BALB/cByJ MEFs. These data suggest that lack of appropriate type I IFN response likely accounts for the higher production of RVF viral particles in MBT/Pas MEFs.

Partial and Delayed Innate Immune Response to RVFV Infection in MBT/Pas MEFs

To validate the microarray data, nine key genes from the IFN-α/β gene induction pathways were chosen: Ifnb1, Ifna4, Rig-I, Stat2, Ifit3/Ifi49, Ifit1, Irf7, Oasl2 and Isg15. Their expression following RVFV infection was studied by quantitative real time RT-PCR (qRT-PCR). We have taken into account the polymorphism between BALB/cByJ and MBT/Pas genomic sequences when designing the PCR primers. Indeed, regions encompassing the amplicons were sequenced in both genomes to exclude any polymorphism in the primers that would influence the PCR efficiency. The TATA box-binding protein gene (Tbp) was chosen to normalize RNA levels because its expression levels were similar in mock- and RVFV-infected MEFs from both genetic backgrounds and remained constant until 9 h after infection (data not shown). However, because NSs viral protein inhibits TFIIH transcription factor starting ˜8 to 9 h post-infection [8], the transcription level of Tbp gene dropped and gene expression was not analyzed at later times.

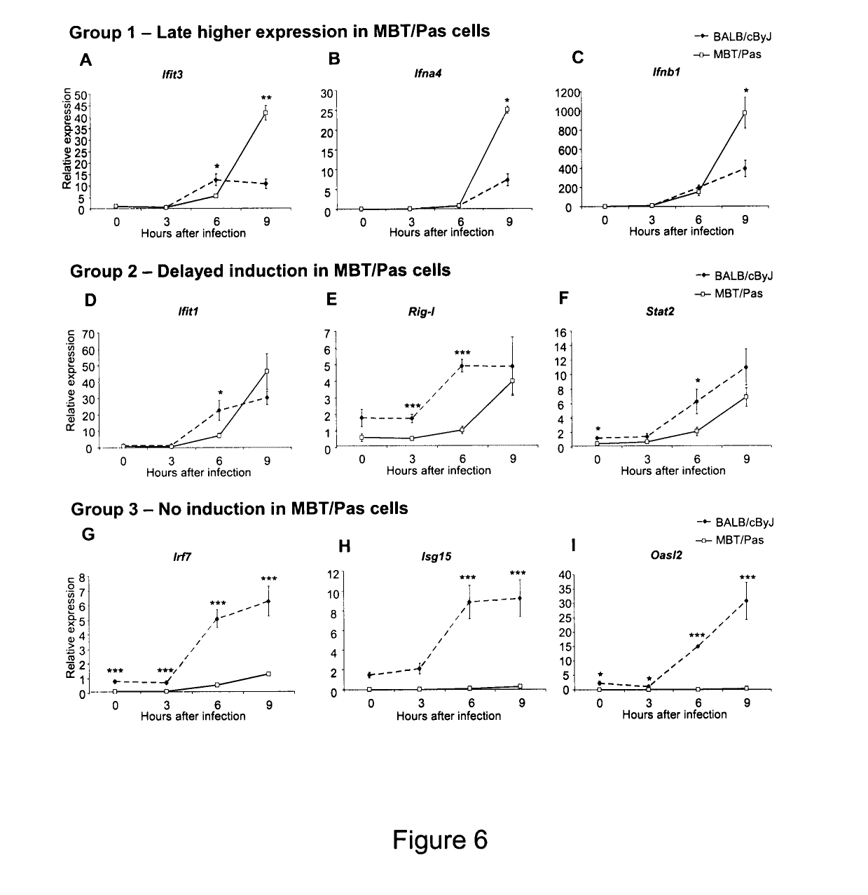

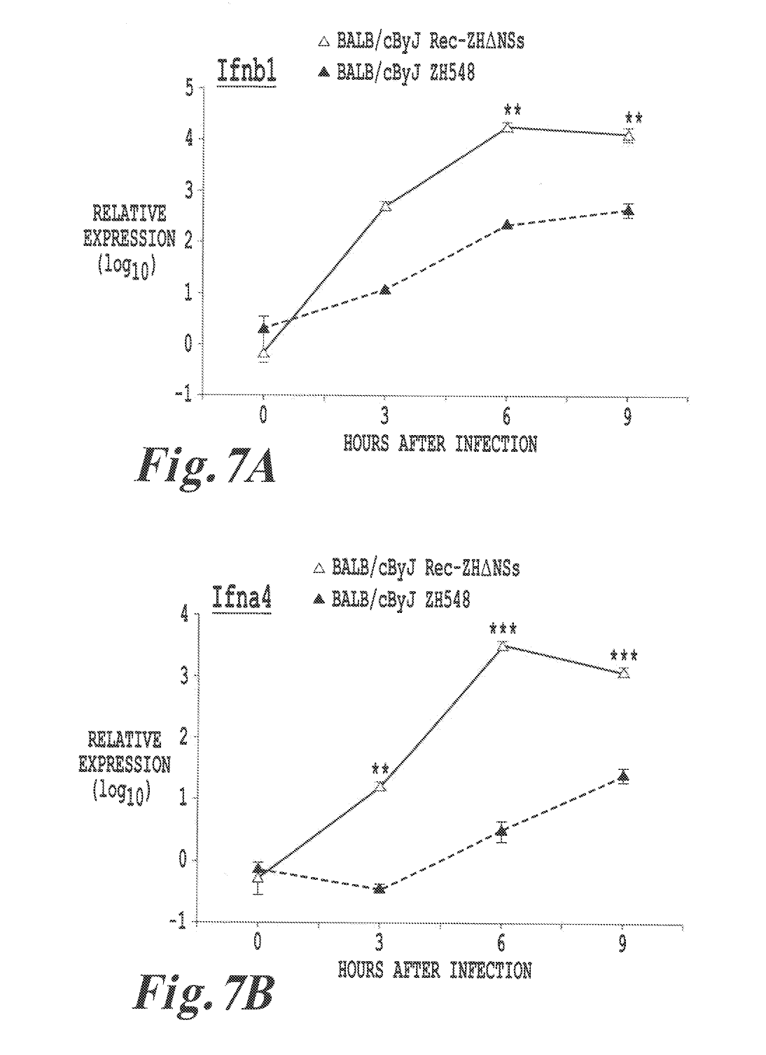

RNAs were extracted at 0, 3, 6 and 9 h post-infection from mock- and RVFV-infected BALB/cByJ and MBT/Pas MEFs. Most selected genes showed congruent and significant difference in transcript levels (FIG. 6). Overall, the signal intensities of DNA microarray hybridization were largely consistent with the results of qRT-PCR, though Ifna4 and Ifnb1 genes were found by qRT-PCR to be upregulated during RVFV infection while microarrays failed to reveal the differential expression, due to the lower sensitivity of microarrays for genes expressed at low levels. According to microarray experiments Isg15 was upregulated by RVFV in both BALB/cByJ and MBT/Pas MEFs, while qRT-PCR showed that Isg15 was highly upregulated in BALB/cByJ MEFs but not in MBT/Pas MEFs. Importantly, the microarray probe for Isg15 is not specific since it also hybridizes with the hypothetical Gm9708 gene, (Affymetrix database; data not shown). Sequencing of the amplicon confirmed the specificity of Isg15 primers, and validated data of the quantitative PCR. Finally, data of the microarray match more closely the quantitative PCR results at 6 h post-infection; it is likely that the course of the viral infections differed slightly in the two successive experiments. Comparison of the levels and kinetics of expression for BALB/cByJ and MBT/Pas MEFs allowed classifying the 9 genes into three groups according to their induction profile. The first profile was observed for Ifit3, Ifna4 and Ifnb1 genes. These genes were expressed at higher levels in MBT/Pas MEFs than in BALB/cByJ MEFs late in infection (FIGS. 6A-C). The second expression profile was observed for Ifit1, Rig-I, and Stat2 genes. These genes had delayed kinetics of induction in MBT/Pas MEFs: their expression was higher in BALB/cByJ MEFs early in infection, but reached similar levels in both cultures at 9 h post-infection (FIG. 6D-F). The last profile consists in genes whose expression was lower in mock-infected MBT/Pas MEFs than in BALB/cByJ MEFs at all times. These genes were either weakly induced or not induced at all in MBT/Pas MEFs. The latter profile was observed for Irf7, Isg15 and Oasl2 genes (FIG. 6G-I). Altogether these results suggest that the MBT/Pas cells elicit a delayed and partial type I IFN response to RVFV infection.

Following infection, RVFV expresses the NSs nonstructural protein. Two of the inventors have shown previously that NSs blocks IFN-β production [9, 34]. Our data demonstrated a significant Ifnb1 gene induction at 6 h post-infection (FIG. 6F). To assess the effect of NSs on type I IFN expression, we used the rec-ZHΔNSs strain, a RVFV derived from wild-type strain ZH548 that carries a deletion in NSs [9]. NSs is non-essential for the virus cycle [5]. BALB/cByJ MEFs were infected with rec-ZHΔNSs and ZH548 strains using a MOI of 5 and the expression of Ifnb1 and Ifna4 genes was examined by qRT-PCR. Kinetic analysis of IFN gene induction showed markedly higher (76- and 1,034-fold, respectively) Ifnb1 and Ifna4 mRNA levels at 6 h post-infection when MEFs were infected with rec-ZHΔNSs than with ZH548 (FIG. 7). Overall, these experiments confirmed the role of NSs as an inhibitor of Ifnb1 gene transcription. They further showed that Ifna4 expression is strongly inhibited when the RVFV genome carries NSs. Nonetheless, efficient production of ISGs may occur in ZH548-infected MEFs because the expression of both Ifnb1 and Ifna4 genes was still significantly stimulated.

Downregulation of Isg15 and Oasl2 Gene Expression Leads to Increased Virus Production

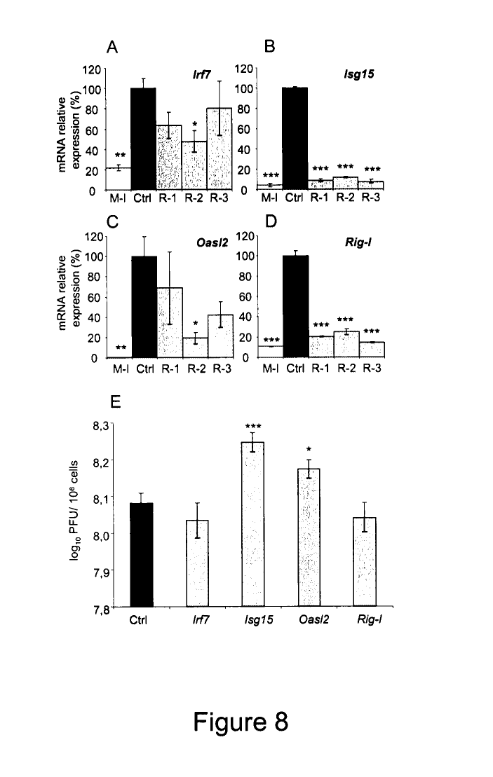

The functional importance of genes that were not induced properly in RVFV-infected MBT/Pas cells was further evaluated. We used small interfering RNAs (siRNAs) to downregulate their expression in BALB/ByJ MEFs and measured the effect of this reduced expression on the viral production. To test whether siRNAs are able to inhibit the expression of RVFV-induced genes in infected MEFs, three stealth siRNAs for Irf7, Isg15, Oasl2 and Rig-I were independently transfected in BALB/ByJ MEFs. Twenty four hours later, the transfected MEFs were infected with RVFV strain rec-ZHΔNSs which triggers a strong interferon response (see FIG. 7). At 6 h post-infection, total RNAs were extracted and Irf7, Isg15, Oasl2 and Rig-I mRNA levels were examined by qRT-PCR. As shown in FIG. 8, the mRNA levels for Irf7, Isg15, Oasl2 and Rig-I were significantly higher in the rec-ZHΔNSs-infected MEFs than in the mock-infected MEFs. Moreover, the mRNA levels for Irf7, Oasl2, Rig-I and Isg15 were lower in at least one of the three specific siRNA-treated cells than in the scramble siRNA-treated cells. Compared with the control, the most efficient siRNA caused 80, 85 and 92% reduction in RNA levels in MEFs for Oasl2, Rig-I and Isg15, respectively. Inhibition of Irf7 expression by the siRNAs though less efficient, about 52% reduction in RNA levels with the most efficient siRNA, was still significant.

Under certain conditions, siRNAs can also trigger the interferon response. It was thus important to exclude the possibility that changes seen in the presence of siRNAs for Irf7, Isg15, Oasl2 and Rig-I could be due to indirect effect of IFN-β induction. BALB/cByJ MEFs were transfected with the most efficient siRNAs for Irf7, Isg15, Oasl2 and Rig-I. Thirty hours after transfection, total RNAs were extracted and Ifnb1 mRNA levels were measured by qRT-PCR. The specific siRNA-treated MEFs did not expressed higher Ifnb1 mRNA levels that MEFs treated with either scramble siRNA or control (no siRNA), indicating that the stealth siRNAs did not stimulate Ifnb1 expression (data not shown).

To detect whether siRNAs targeting Irf7, Isg15, Oasl2 and Rig-I are able to inhibit RVFV production, specific and scramble siRNA were transfected into BALB/cByJ MEFs. Twenty four hours later, the transfected cells were infected with RVFV strain ZH548 at a MOI of 5. The supernatants were harvested 20 h post-infection and were assayed for virus titers. FIG. 8E shows that virus titers were significantly reduced in the supernatants of the MEFs given stealth siRNAs for Isg15 and Oasl2 (p=0.0002 and p=0.0189, respectively). This finding indicates that downregulation of either Isg15 or Oasl2 was effective in inhibiting the production of RVFV strain ZH548, thus demonstrating the functional importance of both genes in the control of infection.

Differences in Susceptibility Among Mouse Inbred Strains.

RVFV infection causes symptoms of various severities in given mammalian species. Furthermore, in contrast with European breeds, indigenous African sheep, goats and cattle may show no clinical signs of illness, despite having a brief period of viraemia [2]. These observations suggest that genetic host components control in part the infection outcome. Experiments in the rat model confirmed the implication of genetics factors in resistance to RVF [14-16], although their nature remains to be identified. Previous investigations in the mouse species did not recognize reproducible differences in the susceptibility to RVF among various inbred strains [13]. This failure might be explained by the limited amount of diversity that segregates among the strains that were challenged. Strains derived from mice trapped in the wild, which represent additional subspecies in the genus Mus, were used in the present study [19]. Our results show that MBT/Pas mice—which belong to the Mus m. musculus subspecies—exhibit an extreme susceptibility to RVF, thus demonstrating phenotypic variability amongst inbred mouse strains.

Stimulation of the Type I Interferon Response.

The inhibition of host cell RNA synthesis induced by the wild-type RVFV ZH548 have previously been identified in cultured cells [8]. In this current study, we used microarrays to examine global gene expression patterns in cells infected with wild-type RVFV ZH548 strain. We identified a set of 229 genes whose expression in BALB/cByJ fibroblasts was modulated in response to infection and implicated IFN-signaling pathways as the predominant biological process induced after RVFV infection. Our results show that at a high MOI, ISGs associated with IFN pathways signaling and innate immune responses were predominant on the list of upregulated genes. A number of mRNA for cytoplasmic recognition receptors that sense nucleic acids were upregulated following MEF infection. Pkr/Eif2ak2 mRNA for the latent dsRNA-dependent PKR was expressed at low levels in BALB/cByJ MEFs and its expression was enhanced about 3-fold following infection. Both Rig-I and Mda5 mRNAs were also upregulated by RVFV infection, RIG-I binds short dsRNA and recognizes the tri-phosphorylated 5′ end of viral ssRNAs [35,36], while MDA5 can bind long dsRNAs [36]. This could suggest that RVFV, like Dengue virus and West Nile virus [37], may be sensed by both RIG-I and MDA5. Indeed, it has been demonstrated that RIG-I plays an important role in sensing RVFV genome. RIG-I binds the 5′ tri-phosphate-containing RNA of RVF virus [35]. Moreover, RIG-I knockdown was shown to decrease the activation of the IFNB1 gene promoter in human 293T cells transfected with genomic RNAs extracted from RVFV particles. In contrast, IFNB1 gene induction was not affected when MDA5 was downregulated [35]. These data would be consistent with the notion that MDA5 activation is not involved in sensing RVFV infection. However, the viral RNA used in the knockdown assays was prepared from RVFV particles while it has been recently shown that the ability to stimulate MDA5 requires the high molecular weight fraction of viral RNAs containing both ssRNA and dsRNA regions present in infected cells [38]. Therefore, we cannot exclude that induction of Mda5 in RVFV-infected MEFs has an essential function and may contribute to the positive feedback regulation of IFN-αs/β production. Alternatively, the induction of Mda5 could merely reflect the fact that in MEFs, Mda5 is an early response gene activated by a synthetic dsRNA, i.e. poly(I:C), in a STAT1-independent manner, and by IFNs [39,40]. The gene for LGP2, the third member of the RIG-like receptor family, which lacks a caspase activation and recruitment domain harbored by RIG-I and MDA5 and therefore cannot activate IRF3 [41], was also upregulated by RVFV infection. Interestingly, expression of Lpg2 gene have been shown to decrease Ifnb1 mRNA production in MEFs when stimulated by a synthetic dsRNA [40]. Lastly, Dai/Zbp1 gene for the cytosolic DNA sensor DAI/ZBP1 was induced in RVFV-infected cells. Dai/Zbp1 is inducible by IFN-β in MEFs [42]. To the best of our knowledge, no viral DNA is generated during RVFV replication. Hence, we believe that the induction of Dai/Zbp1 mRNA in MEFs infected with RVFV has no functional impact on IFNs production.

The Inventors found that the breadth of induced PRR mRNAs was associated with stimulation of a number of virally induced genes. These include major actors of the type I IFN response, as for example Isg15 which was upregulated 9-fold by RVFV in MEFs. Isg15 encodes an ubiquitin-like protein that modifies more than 150 proteins through ISGylation [43]. ISG15 inhibits the degradation of IRF3, thus providing a direct positive loop to stimulate IFN-β expression [44]. ISGs with known antiviral function were also induced, as the genes for the exonuclease ISG20 [28], and for 2′-5′-oligoadenylate synthetases (OAS1A, OASL1, OASL2) [31]. Finally, ISGs whose functions remain largely unknown were upregulated. This is the case for the genes encoding four p65 GTP-binding proteins [29,30] and five p47 GTPases proteins [30]; four of these genes were listed among the top 20 most remarkably induced genes in the infected MEFs. Stimulation of both GTP loading and hydrolysis by Theiler's encephalomyelitis virus infection was recently shown to be, per se, sufficient to stimulate several signaling pathways, though the exact effect of this stimulation on viral replication is not known [45].

Induction of Genes for IFN-αs/β by Rift Valley Fever Virus Infection.

Given the arbitrary cutoff of at least twofold change that we have used to define significant regulated genes in the microarray, genes for IFN-αs/β did not appear as stimulated at 9 h post-infection with RVFV infection in microarrays. With real-time PCR, genes for IFN-β and IFN-α4 were found to be upregulated. These findings likely result from the low abundance of Ifnb1 and Ifna4 mRNAs, which were not detected above background levels on microarrays even after infection, while their moderate but significant stimulation could be measured by real-time PCR. Actually, three mechanisms may account for the low stimulation of type I IFNs, compared for example with the 23- and 41-fold induction of genes for IFN-β and IFN-α4 in West Nile virus-infected MEFs [46]. First, MEFs express Toll-like receptors (TLRs) 1-9 mRNAs and are highly TLR-responsive [47]. However, dsRNA sensing Toll-like receptor 3 (TLR3) was expressed at low levels in RVFV-infected MEFs, in contrast with West Nile virus-infected MEFs (data not shown; [46]. The insignificant role played by TLR3 may contribute in part to the limited induction of genes for IFN-αs/β in infected MEFs. Second, the RVFV NSs protein induces the specific degradation of the dsRNA-dependent PKR, thus attenuating the effects of PKR activation on IFN-β production [6,7]. Third, NSs also interacts with SAP30, YY1 and Sin3A-associated corepressor factors on the Ifnb1 promoter to maintain the gene in a silent repressed state [9,34]. Accordingly, we show here that infection of MEFs with a NSs-null virus induced a more than 70-fold higher Ifnb1 expression compared with wild-type virus. However, mice deficient for IFN-α/β receptor subunit 1 (Ifnar1^{-/-}) were extremely susceptible to RVFV infection, they exhibited enhanced viraemia and earlier lethality than wild-type mice [48]. This last result suggests that, despite the relative low induction of the genes for IFN-β and IFN-α4 in MEFs, type I IFNs still restrict viral spread in vivo. Consistent with this, qRT-PCR experiments revealed that the virulent ZH548 virus was still able to activate Ifnb1 and Ifna4 gene transcription in MEFs, eventually leading to significant expression of ISGs. Therefore, despite the strategies developed by RVFV to escape host defense mechanisms, this Bunyaviridae member virus remains a potent activator of the host innate immune system and an ISG inducer.

The Extreme Susceptibility of MBT/Pas Inbred Strain.

The rapid death of infected MBT/Pas mice within only 4 days post-infection and the three thousand-fold higher production of infectious viral particles in MBT/Pas sera at day 3 post-infection relative to BALB/cByJ sera, suggested that the innate intracellular antiviral response contributes to the MBT/Pas strain susceptibility. The fact that the higher viral production in MBT/Pas mice compared with BALB/cByJ mice could be reproduced in MEFs prompted us to compare the cellular response to RVFV infection in MBT/Pas and BALB/cByJ MEFs by microarrays analysis. Previous experiments have indeed shown that MEFs are excellent model cell lines for analysis of antiviral responses [39,40,42,49]. We found that MBT/Pas cells elicited a weaker interferon response to the viral stress than BALB/cByJ cells. Paradoxically, the genes encoding two key players, IFN-β and IFN-α4, were induced at higher levels in MBT/Pas than in BALB/cByJ cells. The higher production of infectious particles in permissive MBT/Pas cells was likely associated with greater amounts of ligands for PRRs, thus accounting for higher induction of Ifnb1 and Ifna4 mRNAs.

Our analysis of the innate antiviral response to RVFV established that the innate immune response of MBT/Pas MEFs was partial. Indeed, Irf7 mRNA was weakly induced by infection of MBT/Pas cells compared with BALB/cByJ cells. IRF7 plays a critical role within the IFN receptor pathway. IRF7 is required for Ifna4 gene induction and its absence is associated with increased susceptibility to various pathogens such as encephalomyocarditis virus and vesicular stomatitis virus [50]. However, we could not demonstrate a functional role for Irf7 downregulation in viral production. This failure was possibly due to the limited inhibition provided by the siRNA for Irf7. The Oasl2 gene was up-regulated 24-fold after infection in BALB/cByJ MEFs while its expression remained low in MBT/Pas MEFs. The siRNA-mediated downregulation of Oasl2 significantly increased viral production, suggesting that OASL2 is a very potent anti-RVFV effector. The OASL2 protein is active as an OAS [51] and the OASs are known antiviral proteins. Oas1b is actually involved in the innate susceptibility of mice to West Nile virus infection [22,52]. OAS1 is also a genetic determinant of West Nile fever susceptibility in humans [53]. Finally, OAS3 exerts antiviral effects against Chikungunya alphavirus [54]. Isg15 also appeared as critical to restrain RVFV production in MEFs. The increase susceptibility of Isg15-deficient mice to infection with Sinbis virus, influenza virus and HSV-1 suggests that Isg15 is critical for the host response to viral infection [55]. The antiviral effect of ISG15 may be virus-specific, since Isg15-deficient mice exhibited no increase susceptibility to infection with either vesicular stomatitis virus or lymphocytic choriomeningitis virus compared to wild-type mice [56]. Our data suggest a role for the ISG15 ubiquitin-like protein in the antiviral pathway against RVFV infection.

Compared with BALB/cByJ MEFs, the innate immune response of MBT/Pas MEFs to RVFV infection was delayed. Since, the RNA helicase RIG-I drives Ifnb1 promoter activation after RVFV infection [35], Rig-I delayed induction in MBT/Pas cells could contribute to the very low stimulation of Isg15. However, the delayed induction of Rig-I does not account for the weak response of MBT/Pas cells to RVFV since other targets of IRF3, such as Ifit1, were induced similarly in BALB/cByJ and MBT/Pas cells. Moreover, downregulation of Rig-I by a specific siRNA did not lead to an increase viral production in RVFV-infected MEFs. In other terms, Rig-I is not the only gene responsible for the weak interferon response in MBT/Pas MEFs.

In summary, these data suggest that the inability of MBT/Pas cell to limit virus production is the result of several defects in both the early and late phases of the interferon response. These defects causing failure to control the spread of the fast growing RVFV in cultured cells are likely to contribute to the early death of RVFV-infected MBT/Pas mice.

### REFERENCES

1. Flick R, Bouloy M (2005) Rift Valley fever virus. Current Molecular Medicine 5: 827-834.
2. FAO (2003) Recognizing Rift Valley Fever. Rome: Food and agriculture organization of the United Nations. 17. 45 p.
3. Gerdes G H (2004) Rift Valley fever. Rev Sci Tech 23: 613-623.
4. Giorgi C, Accardi L, Nicoletti L, Gro M C, Takehara K, et al. (1991) Sequences and coding strategies of the S RNAs of Toscana and Rift Valley fever viruses compared to those of Punta Toro, Sicilian Sandfly fever, and Uukuniemi viruses. Virology 180: 738-753.
5. Muller R, Saluzzo J F, Lopez N, Dreier T, Turell M, et al. (1995) Characterization of clone 13, a naturally attenuated avirulent isolate of Rift Valley fever virus, which is altered in the small segment. Am J Trop Med Hyg 53: 405-411.
6. Habjan M, Pichlmair A, Elliott R M, Overby A K, Glatter T, et al. (2009) NSs protein of rift valley fever virus induces the specific degradation of the double-stranded RNA-dependent protein kinase. J Virol 83: 4365-4375.
7. Ikegami T, Narayanan K, Won S, Kamitani W, Peters C J, et al. (2009) Rift Valley fever virus NSs protein promotes post-transcriptional downregulation of protein kinase PKR and inhibits eIF2alpha phosphorylation. PLoS Pathog 5: e1000287.
8. Le May N, Dubaele S, Proietti De Santis L, Billecocq A, Bouloy M, et al. (2004) TFIIH transcription factor, a target for the Rift Valley hemorrhagic fever virus. Cell 116: 541-550.
9. Le May N, Mansuroglu Z, Leger P, Josse T, Blot G, et al. (2008) A SAP30 complex inhibits IFN-beta expression in Rift Valley fever virus infected cells. PLoS Pathog 4: e13.
10. Tomori O (1979) Clinical, virological and serological response of the West African dwarf sheep to experimental infection with different strains of Rift Valley fever virus. Res Vet Sci 26: 152-159.
11. Ksiazek T G, Jouan A, Meegan J M, Le Guenno B, Wilson M L, et al. (1989) Rift Valley fever among domestic animals in the recent West African outbreak. Res Virol 140: 67-77.
12. Mims C A (1956) Rift Valley Fever virus in mice. I. General features of the infection. Br J Exp Pathol 37: 99-109.
13. Peters C J, Andeson J (1981) Pathogenesis of Rift Valley Fever. Contr Epidem Biostatist 3: 21-41.
14. Peters C J, Slone T W (1982) Inbred rat strains mimic the disparate human response to Rift Valley fever virus infection. J Med Virol 10: 45-54.
15. Anderson G W, Jr., Rosebrock J A, Johnson A J, Jennings G B, Peters C J (1991) Infection of inbred rat strains with Rift Valley fever virus: development of a congenic resistant strain and observations on age-dependence of resistance. Am J Trop Med Hyg 44: 475-480.
16. Ritter M, Bouloy M, Vialat P, Janzen C, Haller O, et al. (2000) Resistance to Rift Valley fever virus in Rattus norvegicus: genetic variability within certain ‘inbred’ strains. J Gen Virol 81: 2683-2688.
17. Guenet J L, Bonhomme F (2003) Wild mice: an ever-increasing contribution to a popular mammalian model. Trends Genet 19: 24-31.
18. Beck J A, Lloyd S, Hafezparast M, Lennon-Pierce M, Eppig J T, et al. (2000) Genealogies of mouse inbred strains. Nat Genet 24: 23-25.
19. Yang H, Bell T A, Churchill G A, Pardo-Manuel de Villena F (2007) On the subspecific origin of the laboratory mouse. Nat Genet 39: 1100-1107.
20. Bonhomme F, Guénet J L (1996) The laboratory mouse and its wild relatives. In: Lyon M F, Rastan S, Brown S D M, editors. Genetic variants and strains of the laboratory mouse. AVon: Oxford University Press. pp. 1577-1596.
21. El-Akkad A M (1978) Rift Valley fever outbreak in Egypt. October-December 1977. J Egypt Public Health Assoc 53: 123-128.
22. Mashimo T, Lucas M, Simon-Chazottes D, Frenkiel M P, Montagutelli X, et al. (2002) A nonsense mutation in the gene encoding 2′-5′-oligoadenylate synthetase/L1 isoform is associated with West Nile virus susceptibility in laboratory mice. Proc Natl Acad Sci USA 99: 11311-11316.
23. Edgar R, Domrachev M, Lash A E (2002) Gene Expression Omnibus: NCBI gene expression and hybridization array data repository. Nucleic Acids Res 30: 207-210.
24. Sato M, Suemori H, Hata N, Asagiri M, Ogasawara K, et al. (2000) Distinct and essential roles of transcription factors IRF-3 and IRF-7 in response to viruses for IFN-alpha/beta gene induction. Immunity 13: 539-548.
25. Sato M, Taniguchi T, Tanaka N (2001) The interferon system and interferon regulatory factor transcription factors—studies from gene knockout mice. Cytokine & Growth Factor Reviews 12: 133-142.
26. Sato M, Hata N, Asagiri M, Nakaya T, Taniguchi T, et al. (1998) Positive feedback regulation of type I IFN genes by the IFN-inducible transcription factor IRF-7. FEBS Lett 441: 106-110.
27. Grandvaux N, Servant M J, tenOever B, Sen G C, Balachandran S, et al. (2002) Transcriptional profiling of interferon regulatory factor 3 target genes: Direct involvement in the regulation of interferon-stimulated genes. Journal Of Virology 76: 5532-5539.
28. Degols G, Eldin P, Mechti N (2007) ISG20, an actor of the innate immune response. Biochimie 89: 831-835.
29. Itsui Y, Sakamoto N, Kurosaki M, Kanazawa N, Tanabe Y, et al. (2006) Expressional screening of interferon-stimulated genes for antiviral activity against hepatitis C virus replication. J Viral Hepat 13: 690-700.
30. MacMicking J D (2004) IFN-inducible GTPases and immunity to intracellular pathogens. Trends Immunol 25: 601-609.
31. Mashimo T, Simon-Chazottes D, Guenet J L (2008) Innate resistance to flavivirus infections and the functions of 2′-5′ oligoadenylate synthetases. Curr Top Microbiol Immunol 321: 85-100.
32. Martensen P M, Justesen J (2004) Small ISGs coming forward. J Interferon Cytokine Res 24: 1-19.
33. Luan Y, Lengyel P, Liu C J (2008) p204, a p200 family protein, as a multifunctional regulator of cell proliferation and differentiation. Cytokine Growth Factor Rev 19: 357-369.
34. Billecocq A, Spiegel M, Vialat P, Kohl A, Weber F, et al. (2004) NSs protein of Rift Valley fever virus blocks interferon production by inhibiting host gene transcription. J Virol 78: 9798-9806.
35. Habjan M, Andersson I, Klingstrom J, Schumann M, Martin A, et al. (2008) Processing of genome 5′ termini as a strategy of negative-strand RNA viruses to avoid RIG-I-dependent interferon induction. PLoS One 3: e2032.
36. Kato H, Takeuchi O, Mikamo-Satoh E, Hirai R, Kawai T, et al. (2008) Length-dependent recognition of double-stranded ribonucleic acids by retinoic acid-inducible gene-I and melanoma differentiation-associated gene 5. J Exp Med 205: 1601-1610.
37. Saito T, Gale M, Jr. (2007) Principles of intracellular viral recognition. Curr Opin Immunol 19: 17-23.
38. Pichlmair A, Schulz, Tan C P, Rehwinkel J, Kato H, et al. (2009) Activation of MDA5 requires higher order RNA structures generated during virus infection. J Virol.
39. Kang D C, Gopalkrishnan R V, Wu Q, Jankowsky E, Pyle A M, et al. (2002) mda-5: An interferon-inducible putative RNA helicase with double-stranded RNA-dependent ATPase activity and melanoma growth-suppressive properties. Proc Natl Acad Sci USA 99: 637-642.
40. Venkataraman T, Valdes M, Elsby R, Kakuta S, Caceres G, et al. (2007) Loss of DExD/H box RNA helicase LGP2 manifests disparate antiviral responses. J Immunol 178: 6444-6455.
41. Yoneyama M, Kikuchi M, Matsumoto K, Imaizumi T, Miyagishi M, et al. (2005) Shared and unique functions of the DExD/H-box helicases RIG-I, MDA5, and LGP2 in antiviral innate immunity. J Immunol 175: 2851-2858.
42. Takaoka A, Wang Z, Choi M K, Yanai H, Negishi H, et al. (2007) DAI (DLM-1/ZBP1) is a cytosolic DNA sensor and an activator of innate immune response. Nature 448: 501-505.
43. Zhao C, Denison C, Huibregtse J M, Gygi S, Krug R M (2005) Human ISG15 conjugation targets both IFN-induced and constitutively expressed proteins functioning in diverse cellular pathways. Proc Natl Acad Sci USA 102: 10200-10205.
44. Lu G, Reinert J T, Pitha-Rowe I, Okumura A, Kellum M, et al. (2006) ISG15 enhances the innate antiviral response by inhibition of IRF-3 degradation. Cell Mol Biol (Noisy-1e-grand) 52: 29-41.
45. Rubio N, Gonzalez-Tirante M, Arevalo M A, Aranguez I (2008) Over-expression of GTP-binding proteins and GTPase activity in mouse astrocyte membranes in response to Theiler's murine encephalomyelitis virus infection. J Neurochem 104: 100-112.
46. Fredericksen B L, Keller B C, Fornek J, Katze M G, Gale M, Jr. (2008) Establishment and maintenance of the innate antiviral response to West Nile Virus involves both RIG-I and MDA5 signaling through IPS-1. J Virol 82: 609-616.
47. Kurt-Jones E A, Sandor F, Ortiz Y, Bowen G N, Counter S L, et al. (2004) Use of murine embryonic fibroblasts to define Toll-like receptor activation and specificity. J Endotoxin Res 10: 419-424.
48. Bouloy M, Janzen C, Vialat P, Khun H, Pavlovic J, et al. (2001) Genetic evidence for an interferon-antagonistic function of rift valley fever virus nonstructural protein NSs. J Virol 75: 1371-1377.
49. Scherbik S V, Stockman B M, Brinton M A (2007) Differential expression of interferon (IFN) regulatory factors and IFN-stimulated genes at early times after West Nile virus infection of mouse embryo fibroblasts. J Virol 81: 12005-12018.
50. Honda K, Yanai H, Negishi H, Asagiri M, Sato M, et al. (2005) IRF-7 is the master regulator of type-I interferon-dependent immune responses. Nature 434: 772-777.
51. Eskildsen S, Justesen J, Schierup M H, Hartmann R (2003) Characterization of the 2′-5′-oligoadenylate synthetase ubiquitin-like family. Nucleic Acids Res 31: 3166-3173.
52. Perelygin A A, Scherbik S V, Zhulin I B, Stockman B M, Li Y, et al. (2002) Positional cloning of the murine flavivirus resistance gene. Proc Natl Acad Sci USA 99: 9322-9327.
53. Lim J K, Lisco A, McDermott D H, Huynh L, Ward J M, et al. (2009) Genetic variation in OAS1 is a risk factor for initial infection with West Nile virus in man. PLoS Pathog 5: e1000321.
54. Brehin A C, Casademont I, Frenkiel M P, Julier C, Sakuntabhai A, et al. (2009) The large form of human 2′,5′-Oligoadenylate Synthetase (OAS3) exerts antiviral effect against Chikungunya virus. Virology 384: 216-222.
55. Lenschow D J, Lai C, Frias-Staheli N, Giannakopoulos N V, Lutz A, et al. (2007) IFN-stimulated gene 15 functions as a critical antiviral molecule against influenza, herpes, and Sindbis viruses. Proc Natl Acad Sci USA 104: 1371-1376.
56. Osiak A, Utermohlen O, Niendorf S, Horak I, Knobeloch K P (2005) ISG15, an interferon-stimulated ubiquitin-like protein, is not essential for STAT1 signaling and responses against vesicular stomatitis and lymphocytic choriomeningitis virus. Mol Cell Biol 25: 6338-6345.
57. Mroz K, Carrel L, Hunt P A (1999) Germ cell development in the XXY mouse: evidence that X chromosome reactivation is independent of sexual differentiation. Dev Biol 207: 229-238.
58. Irizarry R A, Hobbs B, Collin F, Beazer-Barclay Y D, Antonellis K J, et al. (2003) Exploration, normalization, and summaries of high density oligonucleotide array probe level data. Biostatistics 4: 249-264.
59. Li C, Wong W H (2001) Model-based analysis of oligonucleotide arrays: expression index computation and outlier detection. Proc Natl Acad Sci USA 98: 31-36.
60. Livak K J, Schmittgen T D (2001) Analysis of relative gene expression data using real-time quantitative PCR and the 2(-Delta Delta C(T)) Method. Methods 25: 402-408.
61. Bland J M, Altman D G (2004) The logrank test. Bmj 328: 1073.
62. Hovnanian et al., Genomics, 1998, 52, 267-277.
63. Rebouillat, D. and Hovanessian, A. G., Journal of Interferon and Cytokine Research, 1999, 19, 295-308.
64. Rebouillat et al., Genomics, 2000, 70, 232-240.
65. Justesen et al., Cellular and Molecular Life Sciences, 2000, 57, 1593-1612.
66. Rebouillat; Hovanessian, A. G., Cytokine and Growth Factor Reviews, 2007, 18, 351-361
67. Silverman, J. Virol. 81: 12720, 2007.
68. Hartmann et al., Nucleic Acids Res., 1998, 26, 4121-4128.
69. Rebouillat et al., Eur. J. Biochem., 1998; 257, 319-330.
70. Rebouillat, D. and Hovanessian, A. G., Journal of Interferon and Cytokine Research, 1999, 19, 295-308
71. Hovanessian, A. G., Cytokine and Growth Factor Reviews, 2007, 18, 351-361.
72. Mashimo, T. et al., Proc. Natl. Acad. Sci. USA, 2002, 99, 11311-11316.
73. Lucas et al., Immunol. Cell. Biol., 2003, 81, 230-236.
74. Kajaste-Rudnitski et al., Journal of Biological Chemistry, 2006, 281, 46244637.
75. WO 02/081741
76. Bonnevie-Nielsen et al., Am. J. Hum. Genet., 2005, 76, 623-633.
77. WO 03/089003
78. WO 05/040428
79. St Laurent et al., Cell, 1983, 33, 95-102.
80. Johnston et al., In: Interferon 3: Mechanisms of Production and Action, 1984, 189-298.
81. Friedman, R. M., Ed, Elsevier, Amsterdam.
82. Justesen et al., Proc. Natl. Acad. Sci., USA, 1980, 77, 4618-4622;
84. Justesen et al., Nucleic Acids Res., 1980, 8, 3073-3085.
85. Justesen, J. and Kjelgaard, N. O., Anal. Biochem., 1992, 207, 90-93.
90. Bonetta, The Scientist, 2002, 16, 38.
91. Ford et al., Gene Ther., 2001, 8, 1-4.
92. Wadia and Dowdy, Curr. Opin. Biotechnol., 2002, 13, 52-56.
93. Langel, U. In Handbook of cell penetrating peptides (2nd Ed.), 2006, Lavoisier, FRANCE.

### INCORPORATION BY REFERENCE

Each document, patent, patent application or patent publication cited by or referred to in this disclosure is incorporated by reference in its entirety, especially with respect to the specific subject matter surrounding the citation of the reference in the text. Specific incorporation by reference of references 1 to 93 in the list above is made. However, no admission is made that any such reference constitutes background art and the right to challenge the accuracy and pertinency of the cited documents is reserved.

1. A method for treating a negative-sense single-strand RNA virus infection, comprising:
administering to an individual or subject in need thereof an isolated 2′-5′-oligoadenylate synthetase like protein.
or an isolated polynucleotide encoding said 2′-5′-oligoadenylate synthetase like protein.

2. The method of claim 1, comprising administering an isolated human 2′-5′-oligoadenylate synthetase like protein.

3. The method of claim 1, comprising administering an isolated human 2′-5′-oligoadenylate synthetase like protein.

4. The method of claim 1, wherein said isolated 2′-5′-oligoadenylate synthetase like protein has at least 70% amino acid sequence identity or 80% amino acid sequence similarity to residues 1 to 514 of SEQ ID NO: 5 or residues 1 to 255 of SEQ ID NO: 6.

5. The method of claim 1, wherein said isolated 2′-5′-oligoadenylate synthetase like protein consists of the sequence of mouse 2′-5′-oligoadenylate synthetase-like 2.

6. The method of claim 1, which has at least 70% amino acid sequence identity or 80% amino acid sequence similarity to residues 1 to 508 of SEQ ID NO: 10.

7. The method of claim 1, wherein said negative-sense single-strand RNA virus infection is caused by a virus of the Bunyaviridae family.

8. The method of claim 1, wherein said negative-sense single-strand RNA virus infection is caused by a virus of the Phelobovirus genus.

9. The method of claim 1, wherein said negative-sense single-strand RNA virus infection is caused by Rift Valley Fever Virus.

10. A method for treating a negative-sense single-strand RNA virus infection, comprising:
administering to an individual or subject in need thereof an isolated polynucleotide encoding said 2′-5′-oligoadenylate synthetase like protein.

11. The method of claim 10, comprising comprising administering an isolated polynucleotide encoding a human 2′-5′-oligoadenylate synthetase like protein.

12. The method of claim 10, comprising comprising administering an isolated polynucleotide encoding a murine 2′-5′-oligoadenylate synthetase like protein.

13. The method of claim 10, wherein said isolated polynucleotide encodes a protein having at least 70% amino acid sequence identity or 80% amino acid sequence similarity to residues 1 to 514 of SEQ ID NO: 5 or residues 1 to 255 of SEQ ID NO: 6.

14. The method of claim 10, wherein said isolated polynucleotide encodes a protein having at least 70% amino acid sequence identity or 80% amino acid sequence similarity to residues 1 to 508 of SEQ ID NO: 10.

15. The method of claim 10, wherein said isolated polynucleotide forms a part of an expression vector.

16. The method of claim 10, wherein said negative-sense single-strand RNA virus infection is caused by a virus of the Bunyaviridae family.

17. The method of claim 10, wherein said negative-sense single-strand RNA virus infection is caused by a virus of the Phelobovirus genus.

18. The method of claim 10, wherein said negative-sense single-strand RNA virus infection is caused by Rift Valley Fever Virus.

19. An isolated 2′-5′-oligoadenylate synthetase like protein or an isolated polynucleotide encoding a 2′-5′-oligoadenylate synthetase like protein.

20. A composition comprising the isolated 2′-5′-oligoadenylate synthetase like protein or isolated polynucleotide encoding a 2′-5′-oligoadenylate synthetase like protein of claim 19 in an amount or at a concentration sufficient to prevent or treat a negative-sense single-stranded RNA virus infection, and
a pharmaceutically acceptable carrier, excipient or buffer.

21. A medicinal product or kit comprising:
the isolated 2′-5′-oligoadenylate synthetase like protein or an isolated polynucleotide encoding a 2′-5′-oligoadenylate synthetase like protein of claim 19; and, optionally,
at least one other agent selected from the group consisting of at least one antiviral, anti-inflammatory, or immunomodulatory drug;
wherein, optionally, said medicinal product or kit is formulated for use as a combined preparation for simultaneous, separate or sequential use or administration in the prevention or the treatment of a negative-sense single-stranded RNA virus infection; and
wherein, optionally, said medicinal product or kit contains instructions for using it to prevent or treat a negative-sense single-stranded RNA virus infection.
